(19) United States
(12) Patent Application Publication
(10) Pub. No.: US 2011/0251252 A1
(43) Pub. Date: Oct. 13, 2011

(54) BIOMARKERS FOR MDM2 INHIBITORS FOR USE IN TREATING DISEASE

(75) Inventors: Shaomeng Wang, Saline, MI (US); Sami Malek, Ann Arbor, MI (US); Jianting Long, Ann Arbor, MI (US); Peter Ouillette, Ypsilanti, MI (US)

(73) Assignee: THE REGENTS OF THE UNIVERSITY OF MICHIGAN, Ann Arbor, MI (US)

(21) Appl. No.: 13/082,163

(22) Filed: Apr. 7, 2011

Related U.S. Application Data
(60) Provisional application No. 61/322,592, filed on Apr. 9, 2010.
(60) Provisional application No. 61/451,956, filed on Mar. 11, 2011.

(51) Int. Cl. A61K 31/407; A61P 35/02; C12Q 1/02

(52) U.S. Cl. 514/409; 435/29

(57) ABSTRACT

Provided herein are methods for selecting and treating a subject with leukemia, wherein the subject is selected for treatment and is treated with an MDM2 inhibitor because said subject's cells contain an FLT3-ITD mutation.

### CROSS-REFERENCE TO RELATED APPLICATIONS

The present application claims priority to pending U.S. Provisional Patent Application No. 61/322,592, filed Apr. 9, 2010, and pending U.S. Provisional Patent Application No. 61/451,956, filed Mar. 11, 2011, the contents of which are incorporated herein by reference in their entireties.

### FIELD OF THE INVENTION

Provided herein are methods for identifying and treating leukemia subjects with MDM2 inhibitors.

### BACKGROUND OF THE INVENTION

The aggressive cancer cell phenotype is the result of a variety of genetic and epigenetic alterations leading to deregulation of intracellular signaling pathways. The commonality for all cancer cells, however, is their failure to execute an apoptotic program, and lack of appropriate apoptosis due to defects in the normal apoptosis machinery is a hallmark of cancer. The inability of cancer cells to execute an apoptotic program due to defects in the normal apoptotic machinery is thus often associated with an increase in resistance to chemotherapy, radiation, or immunotherapy-induced apoptosis. Primary or acquired resistance of human cancer of different origins to current treatment protocols due to apoptosis defects is a major problem in current cancer therapy. Accordingly, current and future efforts towards designing and developing new molecular target-specific anticancer therapies to improve survival and quality of life of cancer patients must include strategies that specifically target cancer cell resistance to apoptosis. In this regard, targeting crucial negative regulators that play a central role in directly inhibiting apoptosis in cancer cells represents a highly promising therapeutic strategy for new anticancer drug design.

The p53 tumor suppressor plays a central role in controlling cell cycle progression and apoptosis, and it is an attractive therapeutic target for anticancer drug design because its tumor suppressor activity can be stimulated to eradicate tumor cells (Vogelstein et al., Nature 408:307 (2000)). An approach to stimulating the activity of p53 is through inhibition of its interaction with the protein MDM2 using non-peptide small molecule inhibitors. MDM2 and p53 are part of an auto-regulatory feed-back loop, and MDM2 is transcriptionally activated by p53 and MDM2, in turn, inhibits p53 activity by at least three mechanisms (Wu et al., Genes Dev. 7:1126 (1993). First, MDM2 protein directly binds to the p53 transactivation domain and thereby inhibits p53-mediated transactivation. Second, MDM2 protein contains a nuclear export signal sequence, and upon binding to p53, induces the nuclear export of p53, preventing p53 from binding to the targeted DNAs. Third, MDM2 protein is an E3 ubiquitin ligase and upon binding to p53 is able to promote p53 degradation. Hence, by functioning as a potent endogenous cellular inhibitor of p53 activity, MDM2 effectively inhibits p53-mediated apoptosis, cell cycle arrest and DNA repair. Therefore, small-molecule inhibitors that bind to MDM2 and block the interaction between MDM2 and p53 can promote the activity of p53 in cells with a functional p53 and stimulate p53-mediated cellular effects such as cell cycle arrest, apoptosis, or DNA repair (Chene, Nat. Rev. Cancer 3:102 (2003); Vassilev et al., Science 303:844 (2004)).

The design of non-peptide small-molecule inhibitors that target the p53-MDM2 interaction is currently being pursued as an attractive strategy for anti-cancer drug design (Chene, Nat. Rev. Cancer 3:102 (2003); Vassilev et al., Science 303:844 (2004)). The structural basis of this interaction has been established by x-ray crystallography (Kussie et al., Science 274:948 (1996)).

fms-like tyrosine kinase (FTL3) is a protein of a class III receptor tyrosine kinase (RTK) that is involved in the hematopoietic system (Rosnet, O. et al., Genomics 9:380-385 (1991)). Structurally, RTKs have an extracellular region containing five immunoglobulin-like domains, one juxtamembrane region (JM domain), two tyrosine domains (TK1 and TK2) intervened by a kinase insert domain (KI domain), and the C-terminal domain. A ligand for FLT3 is expressed from stromal cells in the bone marrow, and is present in a membrane-bound or soluble form. This ligand stimulates stem cells independently, or together with other cytokines (Hannum, C. et al., Nature 368: 643-648 (1994)). Therefore, the ligand-receptor interaction between for FL and FLT3 is thought to play an important role in the hematopoietic system. The apoptotic effect of the simultaneous inhibition of mutant FLT3 by the FLT3 inhibitor FI-700 and activation of p53 by the MDM2 inhibitor Nutlin-3 has been reported (Kojima, K. et al., Leukemia 24: 33-43 (2010)).

High levels of FLT3 expression are observed in most of the specimens from patients with acute myeloid leukemia (AML) or acute chronic lymphocytic leukemia (ALL). High levels of FLT3 expression are also found in the patients with chronic myeloid leukemia (CML). FL is known to stimulate the proliferation of AML cells more prominently than AML cells (Piacibello, W. et al., Blood 86: 4105-4114 (1995)).

Somatic mutations in FLT3 were found in AML patients (Nakao, M. et al., Leukemia 10: 1911-1918 (1996)). In these mutants, internal tandem duplication (ITD) was found in the region coding for the JM domain of the FLT3 gene. The duplicated sequences predominantly contain exon 11/12 (now exons 14-15) and intron 20, though varying in length in each sample, and they commonly have an extended JM domain which is translatable in a protein due to an extended in-frame open reading frame. The FLT3 internal tandem duplication (FLT3-ITD) mutation was found in 23% of AML patients (Kottaridis, P. D. et al., Blood 98: 1752 (2010)).

The therapeutic outcome in adult AML remains unsatisfactory, and novel treatment approaches are needed to improve the prognosis of affected patients. One promising approach involves chemical activation of p53 through use of drugs that interfere with the binding of p53 and MDM2 (MDM2 inhibitors): a non-genotoxic approach of inducing cancer cell apoptosis. Various compounds that directly interfere with the binding of p53 and MDM2, including the Nutlins and the MI-series of MDM2 inhibitors, have been developed (Shangary, S, et al., Proc. Natl. Acad. Sci. U.S.A. 105: 3933-3938 (2008); Vassilev, L. T., Trends Mo.l Med. 13:23-31 (2007); Vassilev, L. T. et al., Science 303:844-848 (2004); Ding, K. et al., J. Med. Chem. 49:3432-3435 2006; and Shangary, S. et al., Clin. Cancer Res. 14:5318-5324 (2008)). Currently available evidence indicates that induction of p53 through MDM2 inhibition by Nutlins or MI-series compounds results in the elevation of p53 protein levels, followed by p53-mediated apoptosis or p53/p21-mediated cell cycle arrest (Vassilev, L. T. et al., Science 303:844-848 (2004); Kruse, J. P. et al., Cell. 137:609-622 (2009); Haupt, Y. et al., Nature 387:296-299 (1997); Kubbutat, M. H. et al., Nature 387:299-303 (1997); and Kussie, P. H. et al., Science 274: 948-953 (1996)).

For reasons that remain largely unknown, non-cancerous cells are relatively resistant to MDM2 inhibitor-mediated apoptosis and usually undergo transient cell cycle arrest (Secchiero, P. et al., Blood (2006); Stuhmer, T., et al., Blood 106:3609-3617 (2005)). Equally unclear is the nature and exact contribution of various p53 network/effector molecules to MDM2 inhibitor-induced apoptosis, and thus it remains unknown whether individual p53 effector genes or signaling pathways are absolutely necessary for MDM2 inhibitor-induced apoptosis to occur (Kruse, J. et al., Cell 137:609-622 (2009); Tovar, C. et al., Proc. Natl. Acad. Sci. U.S.A. 103:1888-1893 (2006); Levine, A. J. et al., Nat. Rev. Cancer 9: 749-758 (2009); Villunger, A. et al., Science 302:1036-1038 (2003); Shibue, T. et al., Genes Dev. 17: 2233-2238 (2003)).

Evidence for involvement of intrinsic and extrinsic apoptosis pathways in

MDM2 inhibitor-induced apoptosis, as well as direct effects of the p53 protein on mitochondrial apoptosis molecules has been provided, and it is thus possible that MDM2 inhibitor-mediated apoptosis employs functionally redundant apoptotic pathways (Vaseva, A. V. et al., Cell Cycle 8:1711-1719 (2009); Morselli, E. et al., Cell Cycle 8:1647-1648 (2009); Du, W. et al., J. Biol. Chem. 284: 26315-26321 (2009); (Kojima, K. et al., Blood 108:993-1000 (2006); Kojima, K. et al., Blood 106:3150-3159 (2005); Vousden, K. H. et al., Cell 137: 413-431 (2009); Saddler, C. et al., Blood 111: 1584-1593 (2008)).

Studies into resistance mechanisms to MDM2 inhibitors in various cell systems have shown that intact p53 may be needed for MDM2 inhibitor-induced apoptosis to occur (Secchiero, P. et al., Blood 107: 4122-4129 (2006); Saddler, C. et al., Blood 111: 1584-1593 (2008); Coll-Mulet, L. et al., Blood 107: 4109-4114 (2006)). What is less clear is how often and under what cellular circumstances wild type p53 status is alone sufficient as a predictor for sensitivity, or what other sensitivity/resistance determinants may be operational (Secchiero, P. et al., Blood 113: 4300-4308 (2009); Kitagawa, M. et al., Oncogene 27: 5303-5314 (2008); Kitagawa, M. et al., Mol. Cell. 29: 217-231 (2008)). Two of the proposed regulators of p53-mediated apoptosis are MDM2 and MDMX, and elevated levels of these proteins have been shown to influence MDM2 inhibitor sensitivities in various experimental settings. Nonetheless, the available evidence in support of a critical role of these proteins is neither conclusive nor consistent across experimental systems, and thus it remains possible that these p53 regulatory molecules are not critical determinants of MDM2 inhibitor efficaciousness in all human tumors (Laurie, N. A. et al., Nature 444: 61-66 (2006); Hu, B. et al., J. Biol. Chem. 281: 33030-33035 (2006); Francoz, S. et al., Proc. Natl. Acad. Sci. U.S.A. 103: 3232-3237 (2006)).

There remains a need to be able to predict which leukemia patients are likely to benefit from MDM2 inhibitor therapy.

### BRIEF SUMMARY OF THE INVENTION

Provided herein are methods of selecting a human subject for treatment of leukemia. In some embodiments, the method comprises (a) determining whether the subject's cells contain an FLT3-ITD mutation, and (b) selecting the subject for treatment for leukemia if the cells contain the mutation.

Also provided are methods of treating leukemia. In some embodiments, the method comprises administering a MDM2 inhibitor to a human subject with leukemia in whom the subject's cells contain an FLT3-ITD mutation.

Also provided are methods of selecting a human subject for treatment of leukemia. In some embodiments, the method comprises testing the cells of the subject for the presence of a FLT3-ITD mutation.

Also provided are methods of predicting treatment outcomes in a human subject having leukemia. In some embodiments administering a MDM2 inhibitor to a subject having a FLT3-ITD mutation will increase the likelihood of generating a favorable therapeutic response in the subject.

### BRIEF DESCRIPTION OF THE DRAWINGS

FIGS. 1A and 1B are line graphs that depict resistance of AML blasts to the MDM2 inhibitors MI-219 (FIG. 1A) and MI-63 (FIG. 1B). One hundred and nine (MI-219) and 60 (MI-63) AML samples were enriched to >90% blast purity through negative selection and incubated for 40h with various concentrations of either MI-219 or MI-63. Samples were prepared for annexin-V and PI staining and analyzed by flow cytometry, and the residual live and non-apoptotic cell fraction was calculated for each concentration by comparison with the untreated control aliquots. A. MI-219 assay results. Red: p53 sequence mutants; green: cases with absent p53 mRNA; black: wild type p53 status. B. MI-63 assay results. Red: p53 sequence mutants; green: cases with absent p53 mRNA; black: wild type p53 status.

FIG. 2 is a line graph that depicts sensitivities of 19 AML cell lines to MI-219.

FIGS. 3A and 3B are immunoblots that depict wild-type and mutant p53 levels in primary AML blasts after treatment with MI-219, Nutlin3 or external irradiation. AML blasts were purified through negative selection and either left untreated or treated for 8 hours with MI-219 (5 μM), Nutlin 3 (5 μM) or one-time external irradiation (5 Gy). After 8 hours, cells were lysed and protein fractionated by SDS-PAGE. Each gel was also loaded with an aliquot of a MOLM 13 AML cell line lysate as an internal standard (loaded as 1.25, 2.5 and 5 μg of MI-219-treated lysate or 5 μg of untreated lysate (UT), respectively). Protein was transferred to membrane and prepared for immunoblotting with an anti-p53 and anti-actin antibody. Films for both, p53 and actin were developed together. IC₅₀ values for MI-219 are indicated in brackets.

FIGS. 4A and 4B are dot graphs that depict the levels of MDM2 mRNA (FIG. 4A) and mRNA MDMX (FIG. 4B) in AML blasts treated with MI-219. Normalized expression levels of MDM2 and MDMX mRNA were measured in cDNA made from RNA from FACS-sorted AML blasts. Displayed are delta Ct values (Ct mean MDM2 or MDMx−Ct mean PGK1) grouped by MI-219 IC₅₀ values as indicated. Red diamonds indicated AML blasts with mutated p53.

FIGS. 5A and 5B are immunoblots that depict p53 level in AML patient buccal samples (FIG. 5A) and blast samples (FIG. 5B). FIG. 5C is an LOH analysis. FIG. 5D is a table that sets forth the p53 mutation analysis. FIG. 5E is a dot graph that depicts p53 expression by QPCR. Files generated through use of the Affymetrix program Genotyping Console for all patients were imported into the LOH tool version 2 using software tool PLUT, and all individual positions of LOH between buccal DNA and paired tumor DNA were graphed as a blue tick mark across the length of the chromosomes. Copy number estimates for all SNP positions for all patients were generated through dChipSNP, as described, and displayed across the length of the chromosomes. Copy losses are displayed with blue colors, copy gains with red colors. A,B: Heatmap display of chromosomal copy number changes at 17p based on SNP 6.0 array profiling. Blue: copy loss; red: copy gain. A: Buccal DNA, B: AML blast DNA. C: LOH analysis at 17p comparing paired blasts and buccal DNA. Red numbering: Copy-neutral LOH (acquired uniparental disomy); black: LOH with copy loss. D: p.53 exon 5-9 mutation analysis results. E: Normalized p53 mRNA expression in AML blasts grouped by MI-219 IC₅₀ values as indicated. Red diamonds indicate AML blasts with mutated p53.

FIG. 6 is a dot graph that depicts the sensitivity of AML blasts having various p53 mutations to the MDM2 inhibitor MI-219. Display of MI-219 IC₅₀ values categorized by 1) p53 mutation status, ii) presence of FLT3-ITD and iii) all others.

Differences in the mean IC₅₀ value between FLT3-ITD+ and all other cases are significant (p=0.02).

### DETAILED DESCRIPTION OF THE INVENTION

The survival of most patients with acute myelogenous leukemia (AML) remains poor, and novel therapeutic approaches are needed to improve outcomes. Results of a detailed characterization of sensitivity of leukemic cells to MDM2 inhibitors are described herein. In one embodiment, the leukemia is acute lymphatic (ALL). In one embodiment, the leukemia is chronic myeloid (CML). In another embodiment, the leukemia that is treated is acute myeloid leukemia (AML).

As used herein, the terms “acute myeloid leukemia (AML)” and “acute myelogenous leukemia” are synonymous.

In another embodiment, the acute myeloid leukemia is type M0 (minimally differentiated acute myeloblastic leukemia). In another embodiment, the acute myeloid leukemia is type M1 (acute myeloblastic leukemia, without maturation). In another embodiment, the acute myeloid leukemia is type M2 (acute myeloblastic leukemia, with granulocytic maturation). In another embodiment, the acute myeloid leukemia is type M3 (promyelocytic or acute promyelocytic leukemia). In another embodiment, the acute myeloid leukemia is type M4 (acute myelomonocytic leukemia). In another embodiment, the acute myeloid leukemia is type M4eo (myelomonocytic together with bone marrow eosinophilia). In another embodiment, the acute myeloid leukemia is type M5a (acute monoblastic leukemia). In another embodiment, the acute myeloid leukemia is type M5b (acute monocytic leukemia). In another embodiment, the acute myeloid leukemia is type M6 (acute erythroid leukemia). In another embodiment, the acute myeloid leukemia is type M6a (erythroleukemia). In another embodiment, the acute myeloid leukemia is type M6b (very rare erythroid leukemia). In another embodiment, the acute myeloid leukemia is type M7 (acute megakaryoblastic leukemia). In another embodiment, the acute myeloid leukemia is type M8 (acute basophilic leukemia). In another embodiment, the acute myeloid leukemia is acute basophilic leukemia. In another embodiment, the acute myeloid leukemia is acute eosinophilic leukemia. In another embodiment, the acute myeloid leukemia is mast cell leukemia. In another embodiment, the acute myeloid leukemia is acute myeloid dendritic cell leukemia. In another embodiment, the acute myeloid leukemia is acute panmyelosis with myelofibrosis. In another embodiment, the acute myeloid leukemia is myeloid sarcoma.

In one embodiment, the cells are leukemia cells. In another embodiment, the cells are acute myeloid leukemia cells.

In one aspect, the disclosure relates to personalized medicine for patients having leukemia, and encompasses the selection of treatment options with the highest likelihood of successful outcome for individual leukemia patients. In another aspect, the disclosure relates to the use of an assay(s) to predict the treatment outcome, e.g., the likelihood of favorable responses or treatment success, in patients having leukemia.

Provided herein are methods of selecting a patient, e.g., human subject for treatment of leukemia with an MDM2 inhibitor, comprising obtaining a biological sample, e.g., blood cells, from the patient, testing a biological sample from the patient for the presence of a biomarker, e.g., a FLT3 having an activating mutation, and selecting the patient for treatment if the biological sample contains a FLT3 having an activating mutation. In one embodiment, the methods further comprise administering a therapeutically effective amount of an MDM2 inhibitor to the patient if the biological sample contains a FLT3 activating mutation. Examples of FLT3 activating mutations include, for example, FLT3-ITD (internal tandem duplication) and FTL3-KD (tyrosine kinase domain) mutations.

Provided herein are methods of predicting treatment outcomes in a patient having leukemia, comprising obtaining a biological sample, from the patient, testing the biological sample from the patient for the presence of a FLT3 having an activating mutation, wherein the detection of an activating mutation indicates the patient will respond favorably to administration of a therapeutically effective amount of an MDM2 inhibitor. Favorable responses include, but are not limited to, hematologic responses, e.g., normalization of blood counts in the patient—white blood cells, red blood cells, and platelets (detectable by simple blood tests); cytogenetic responses, e.g., reduction or disappearance of the number of Philadelphia chromosome-positive cells in the patient (detectable by standard laboratory methods) and/or molecular responses, e.g., reduction or disappearance in quantities of the abnormal BCR-ABL protein in the patient (detectable by PCR assays).

Provided herein are methods of treating leukemia, comprising administering a therapeutically effective amount of a MDM2 inhibitor to a patient, e.g., a human subject, with leukemia in whom the patient's cells contain a FLT3 having an activating mutation. In one embodiment, the patient is selected for treatment with the MDM2 inhibitor after the patient's cells have been determined to contain an FLT3-ITD mutation. In one embodiment, the method of treating a patient having leukemia comprises obtaining a biological sample from the patient, determining whether the biological sample contains a FLT3 having an activating mutation, and administering to the patient a therapeutically effective amount of an MDM2 inhibitor, e.g., a compound of Chart 1, if the biological sample contains a FLT3 having an activating mutation.

In another embodiment, the methods provided herein further comprise determining whether the patient's cells contain a p53 mutation.

The term “biomarker” as used herein refers to any biological compound, such as a protein, a fragment of a protein, a peptide, a polypeptide, a nucleic acid, etc. that can be detected and/or quantified in a patient in vivo or in a biological sample obtained from a patient. Furthermore, a biomarker can be the entire intact molecule, or it can be a portion or fragment thereof. In one embodiment, the expression level of the biomarker is measured. The expression level of the biomarker can be measured, for example, by detecting the protein or RNA (e.g., mRNA) level of the biomarker. In some embodiments, portions or fragments of biomarkers can be detected or measured, for example, by an antibody or other specific binding agent. In some embodiments, a measurable aspect of the biomarker is associated with a given state of the patient, such as a particular stage of cancer. For biomarkers that are detected at the protein or RNA level, such measurable aspects may include, for example, the presence, absence, or concentration (i.e., expression level) of the biomarker in a patient, or biological sample obtained from the patient. For biomarkers that are detected at the nucleic acid level, such measurable aspects may include, for example, allelic versions of the biomarker or type, rate, and/or degree of mutation of the biomarker, also referred to herein as mutation status.

For biomarkers that are detected based on expression level of protein or RNA, expression level measured between different phenotypic statuses can be considered different, for example, if the mean or median expression level of the biomarker in the different groups is calculated to be statistically significant. Common tests for statistical significance include, among others, t-test, ANOVA, Kruskal-Wallis, Wilcoxon, Mann-Whitney, Significance Analysis of Microarrays, odds ratio, etc. Biomarkers, alone or in combination, provide measures of relative likelihood that a subject belongs to one phenotypic status or another. Therefore, they are useful, inter alia, as markers for disease and as indicators that particular therapeutic treatment regimens will likely result in beneficial patient outcomes.

In one embodiment of the disclosure, the biomarker is the FLT3 receptor (also referred to herein as FLT3). In one embodiment of the disclosure, the measurable aspect of the FLT3 receptor is mutation status. In one embodiment of the disclosure, the mutation status is one that results in increased tyrosine kinase activity of the FLT3 receptor and/or constitutive activation of the FLT3 receptor tyrosine kinase. Such mutations include, for example, one or more internal tandem duplications (ITD) of the juxtamembrane domain and/or one or more mutations in the tyrosine kinase domain (TKD).

Thus, in certain aspects of the disclosure, the biomarker is FLT3 which is differentially present in a subject of one phenotypic status (e.g., a patient having cancer, e.g., leukemia, with mutation-bearing cells) as compared with another phenotypic status (e.g., a normal undiseased patient or a patient having cancer without mutation-bearing cells).

In addition to individual biological compounds (e.g., FLT3, p53), the term “biomarker” as used herein is meant to include groups or sets of multiple biological compounds. For example, the combination of FLT3 and p53 may comprise a biomarker. Thus, a “biomarker” may comprise one, two, three, four, five, six, seven, eight, nine, ten, fifteen, twenty, twenty five, thirty, or more, biological compounds.

The determination of the expression level or mutation status of a biomarker in a patient can be performed using any of the many methods known in the art. Any method known in the art for quantitating specific proteins and/or detecting FLT3 and/or p53 mutations in a patient or a biological sample may be used in the methods of the disclosure. Examples include, but are not limited to, PCR (polymerase chain reaction), or RT-PCR, Northern blot, Western blot, ELISA (enzyme linked immunosorbent assay), RIA (radioimmunoassay), gene chip analysis of RNA expression, immunohistochemistry or immunofluorescence (See, e.g., Slagle et al. Cancer 83:1401 (1998)). Certain embodiments of the disclosure include methods wherein biomarker RNA expression (transcription) is determined. Other embodiments of the disclosure include methods wherein protein expression in the biological sample is determined. See, for example, Harlow et al., Antibodies: A Laboratory Manual, Cold Spring Harbor Laboratory, Cold Spring Harbor, N.Y., (1988) and Ausubel et al., Current Protocols in Molecular Biology, John Wiley & Sons, New York 3rd Edition, (1995). For northern blot or RT-PCR analysis, RNA is isolated from the tumor tissue sample using RNAse free techniques. Such techniques are commonly known in the art.

When quantified in a patient in vivo, the expression level of proteins such as FLT3 or variants thereof may be determined by administering an antibody that binds specifically to FLT3 (See, e.g., U.S. Published Appl. No. 2006/0127945) and determining the extent of binding. The antibody may be detectably labeled, e.g., with a radioisotope such as carbon-11, nitrogen-13, oxygen-15, and fluorine-18. The label may then be detected by positron emission tomography (PET).

In one embodiment of the disclosure, a biological sample is obtained from the patient and cells in the biopsy are assayed for determination of biomarker expression or mutation status.

In one embodiment of the disclosure, PET imaging is used to determine biomarker expression.

In another embodiment of the disclosure, Northern blot analysis of biomarker transcription in a tumor cell sample is performed. Northern analysis is a standard method for detection and/or quantitation of mRNA levels in a sample. Initially, RNA is isolated from a sample to be assayed using Northern blot analysis. In the analysis, the RNA samples are first separated by size via electrophoresis in an agarose gel under denaturing conditions. The RNA is then transferred to a membrane, crosslinked and hybridized with a labeled probe. Typically, Northern hybridization involves polymerizing radiolabeled or nonisotopically labeled DNA, in vitro, or generation of oligonucleotides as hybridization probes. Typically, the membrane holding the RNA sample is prehybridized or blocked prior to probe hybridization to prevent the probe from coating the membrane and, thus, to reduce non-specific background signal. After hybridization, typically, unhybridized probe is removed by washing in several changes of buffer. Stringency of the wash and hybridization conditions can be designed, selected and implemented by any practitioner of ordinary skill in the art. Detection is accomplished using detectably labeled probes and a suitable detection method. Radiolabeled and non-radiolabled probes and their use are well known in the art. The presence and or relative levels of expression of the biomarker being assayed can be quantified using, for example, densitometry.

In another embodiment of the disclosure, biomarker expression and/or mutation status is determined using RT-PCR. RT-PCR allows detection of the progress of a PCR amplification of a target gene in real time. Design of the primers and probes required to detect expression and/or mutation status of a biomarker of the disclosure is within the skill of a practitioner of ordinary skill in the art. RT-PCR can be used to determine the level of RNA encoding a biomarker of the disclosure in a tumor tissue sample. In an embodiment of the disclosure, RNA from the biological sample is isolated, under RNAse free conditions, than converted to DNA by treatment with reverse transcriptase. Methods for reverse transcriptase conversion of RNA to DNA are well known in the art. A description of PCR is provided in the following references: Mullis et al., Cold Spring Harbor Symp. Quant. Biol. 51:263 (1986); EP 50,424; EP 84,796; EP 258,017; EP 237,362; EP 201,184; U.S. Pat. Nos. 4,683,202; 4,582,788; 4,683,194.

RT-PCR probes depend on the 5′-3′ nuclease activity of the DNA polymerase used for PCR to hydrolyze an oligonucleotide that is hybridized to the target amplicon (biomarker gene). RT-PCR probes are oligonucleotides that have a fluorescent reporter dye attached to the 5, end and a quencher moiety coupled to the 3′ end (or vice versa). These probes are designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. This decouples the fluorescent and quenching dyes and FRET no longer occurs. Thus, fluorescence increases in each cycle, in a manner proportional to the amount of probe cleavage. Fluorescence signal emitted from the reaction can be measured or followed over time using equipment which is commercially available using routine and conventional techniques.

In still another embodiment of the disclosure, expression of proteins encoded by biomarkers are detected by western blot analysis. A western blot (also known as an immunoblot) is a method for protein detection in a given sample of tissue homogenate or extract. It uses gel electrophoresis to separate denatured proteins by mass. The proteins are then transferred out of the gel and onto a membrane (e.g., nitrocellulose or polyvinylidene fluoride (PVDF)), where they are detected using a primary antibodythat specifically bind to the protein. The bound antibody can then detected by a secondary antibody that is conjugated with a detectable label (e.g., biotin, horseradish peroxidase or alkaline phosphatase). Detection of the secondary label signal indicates the presence of the protein.

In still another embodiment of the disclosure, the expression of a protein encoded by a biomarker is detected by enzyme-linked immunosorbent assay (ELISA). In one embodiment of the disclosure, “sandwich ELISA” comprises coating a plate with a capture antibody; adding sample wherein any antigen present binds to the capture antibody; adding a detecting antibody which also binds the antigen; adding an enzyme-linked secondary antibody which binds to detecting antibody; and adding substrate which is converted by an enzyme on the secondary antibody to a detectable form. Detection of the signal from the secondary antibody indicates presence of the biomarker antigen protein.

In still another embodiment of the disclosure, the expression of a biomarker is evaluated by use of a gene chip or microarray. Such techniques are within ordinary skill held in the art.

The term “biological sample” as used herein refers any tissue or fluid from a patient that is suitable for detecting a biomarker, such as FLT3-ITD mutation status. Examples of useful biological samples include, but are not limited to, biopsied tissues and/or cells, e.g., solid tumor, lymph gland, inflamed tissue, tissue and/or cells involved in a condition or disease, blood, plasma, serous fluid, cerebrospinal fluid, saliva, urine, lymph, cerebral spinal fluid, and the like. Other suitable biological samples will be familiar to those of ordinary skill in the relevant arts. A biological sample can be analyzed for biomarker expression and/or mutation using any technique known in the art and can be obtained using techniques that are well within the scope of ordinary knowledge of a clinical practioner. In one embodiment of the disclosure, the biological sample comprises blood cells.

As used herein, an “MDM2 inhibitor” is a compound that interferes with MDM2 activity. MDM2 inhibitors are well known to those of ordinary skill in the art. For example, see Shangary, S. et al., Annual Review Of Pharmacology and Toxicology 49: 223-241 (2009); and Weber, L. Expert Opinion On Therapeutic Patents 20: 179-191 (2010).

In another embodiment, the MDM2 inhibitor is a spiro-oxindole compound. As used herein, the term “spiro-oxindole MDM2 inhibitor” refers, for example, to a compound disclosed in U.S. Patent Application Nos. 61/260,685; 61/263,662; 61/413,094, 61/451,968, 11/360,485 (US 2006/0211757 A1); Ser. No. 11/848,089 (US 2008/0125430 A1); or Ser. No. 12/945,511, or in International Patent Application Nos. PCT/US2006/0062 (WO 2006/091646) or PCT/US2007/019128 (WO 2008/036168). In a particular embodiment, the spiro-oxindole MDM2 inhibitor is a compound of Chart 1. In another particular embodiment, the spiro-oxindole MDM2 inhibitor is a compound of Chart 2. The compounds of Chart 1 bind to human MDM2 protein with high affinities in fluorescence-polarization based biochemical binding assay, effectively activate p53 and induce cell growth inhibition and cell death in tumor cells with wild-type p53. Significantly, these compounds are capable of inhibiting tumor growth in xenograft models of human cancer, suggesting that these compounds hold promise as new anticancer drugs.

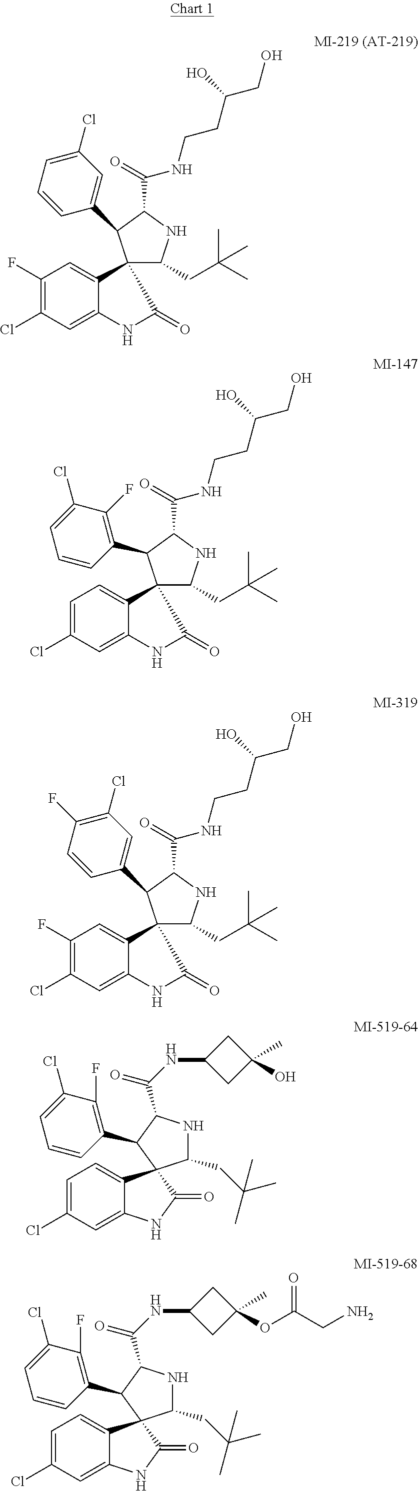

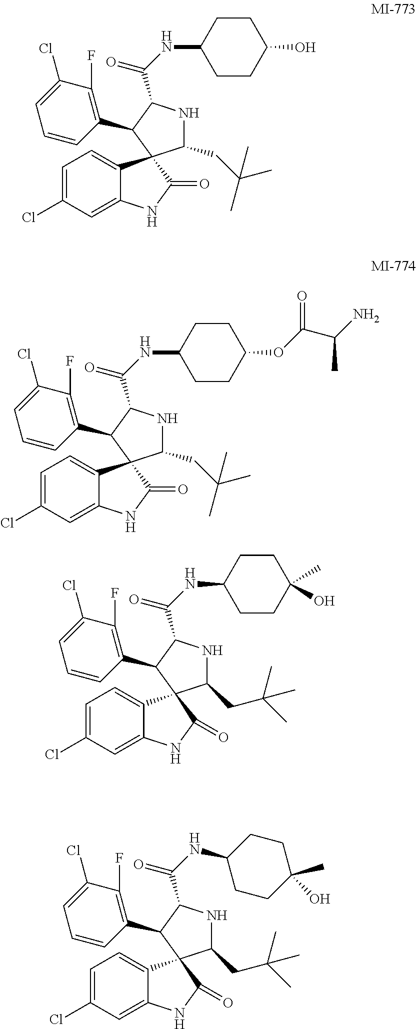

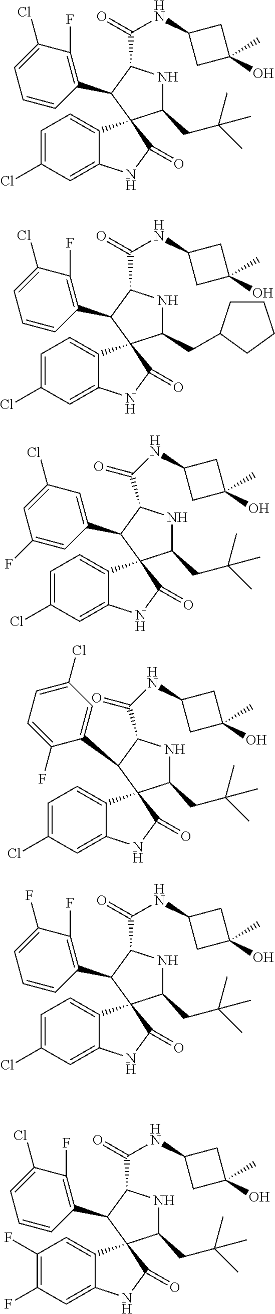

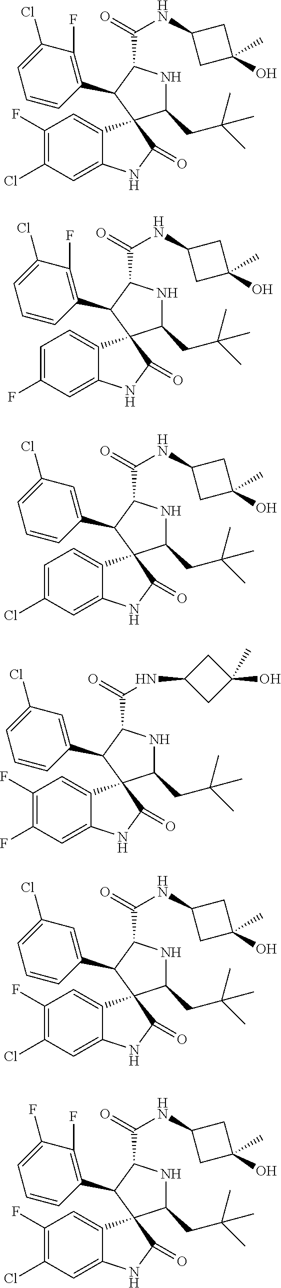

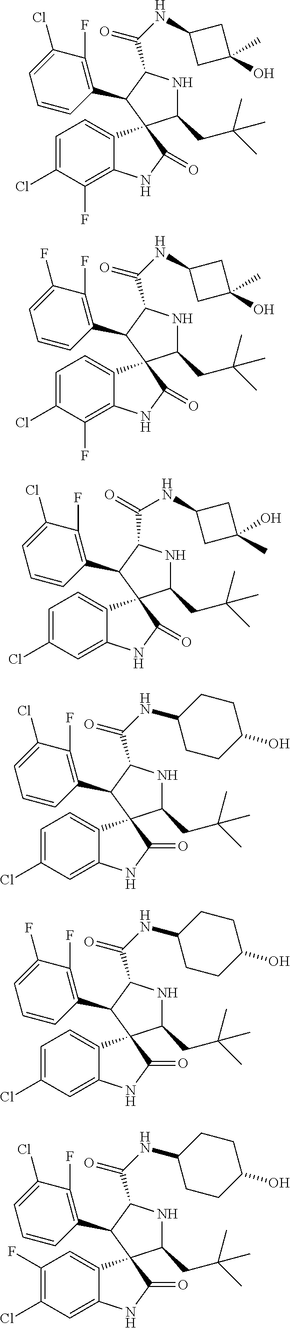

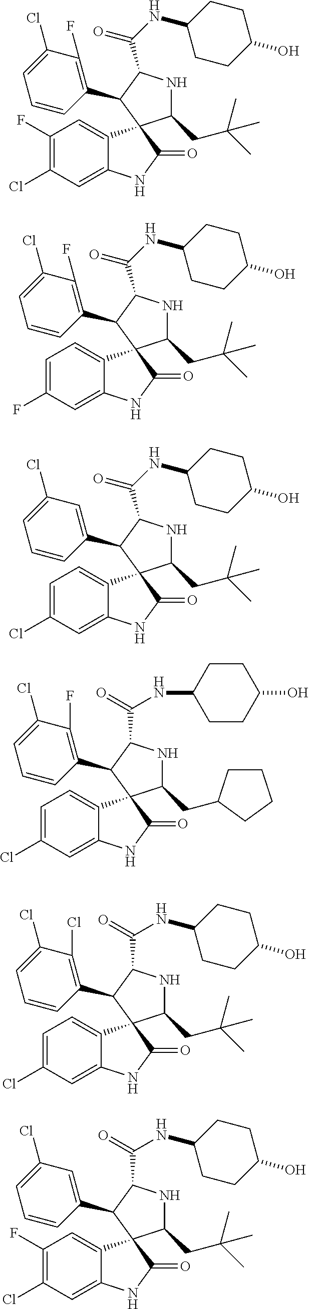

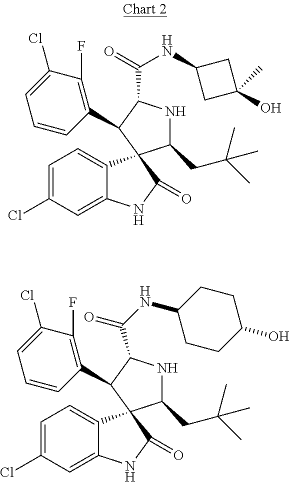

In another embodiment, the MDM2 inhibitor is a cis-imidazoline compound. As used herein, the term “cis-imidazoline MDM2 inhibitor” refers, for example, to a compound disclosed in U.S. Pat. Nos. 6,617,346; 6,734,302; 7,132,421; 7,425,638; or 7,579,368; or U.S. Patent Application Publication Nos. 2005/0288287 or U.S. 2009/0143364. A cis-imidazoline MDM2 inhibitor is commonly referred to as a “nutlin.” In a particular embodiment, the cis-imidazoline is Nutlin-1, Nutlin-2, or Nutlin-3 (Chart 3; see Vassilev, L. T. et al., Science 303:844-848 (2004)).

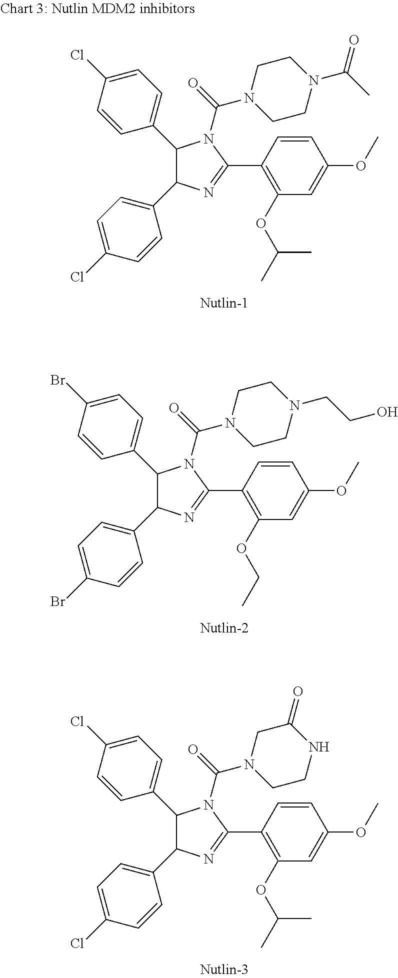

In another embodiment, the MDM2 inhibitor is a substituted piperidine compound. As used herein, the term “substituted piperidine MDM2 inhibitor” refers, for example, to a compound disclosed in U.S. Pat. Nos. 7,060,713 or 7,553,833.

In another embodiment, the MDM2 inhibitor is a spiroindolinone compound. As used herein, the term “spiroindolinone MDM2 inhibitor” refers, for example, to a compound disclosed in U.S. Pat. Nos. 6,916,833; 7,495,007; or 7,638,548.

In another embodiment, the MDM2 inhibitor is an oxindole compound. As used herein, the term “oxindole MDM2 inhibitor” refers, for example, to a compound disclosed in U.S. Pat. No. 7,576,082.

In another embodiment, the MDM2 inhibitor is a diphenyl-dihydro-imidazopyridinone compound. As used herein, the term “diphenyl-dihydro-imidazopyridinone MDM2 inhibitor” refers, for example, to a compound disclosed in U.S. Pat. No. 7,625,895.

In another embodiment, the MDM2 inhibitor is an imidazothiazole compound. As used herein, the term “imidazothiazole MDM2 inhibitor” refers, for example, to a compound disclosed in U.S. 2009/0312310.

In another embodiment, the MDM2 inhibitor is a deazaflavin compound. As used herein, the term “deazaflavin MDM2 inhibitor” refers, for example, to a compound disclosed in U.S. Patent Application Publication Nos. 2006/0211718 or 2010/0048593.

In another embodiment, the MDM2 inhibitor is a benzodiazapine compound. As used herein, the term “benzodiazapine MDM2 inhibitor” refers, for example, to a compound disclosed in U.S. 2005/0227932.

In another embodiment, the MDM2 inhibitor is an isoindolin-1-one compound. As used herein, the term “isoindolin-1-one MDM2 inhibitor” refers, for example, to a compound disclosed in U.S. 2008/0261917.

In another embodiment, the MDM2 is a boronic acid. As used herein, the term “boronic acid MDM2 inhibitor” refers, for example, to a compound disclosed in U.S. Patent Application Publication Nos. 2009/0227542 or 2008/0171723.

In another embodiment, the MDM2 inhibitor is a peptide or polypeptide. As used herein, the term “peptidic MDM2 inhibitor” refers for example, to a compound disclosed in U.S. Pat. No. 7,083,983; U.S. 2006/0211757 A1; U.S. 2005/0137137; U.S. 2002/0132977; U.S. 2009/0030181; or WO 2008/106507.

In another embodiment, the MDM2 inhibitor is a compound disclosed in any of Shangary, S, et al., Proc. Natl. Acad. Sci. USA. 105:3933-3938 (2008); Vassilev, L. T., Trends Mol. Med. 13:23-31 (2007); Vassilev, L. T. et al., Science 303:844-848 (2004); Ding, K. et al., J. Med. Chem. 49:3432-3435 2006; Shangary, S. et al., Clin. Cancer Res. 14:5318-5324 (2008); Chene, P., Molecular Cancer Research 2:20-28 (2004); Pazgier et al., Proc. Natl. Acad. Sci. USA. 106:4665-4670 (2009); U.S. 2008/0280769; U.S. 008/0039472; U.S. 2009/0149493; or U.S. 2004/0171035.

In another embodiment, the MDM2 inhibitor is a compound disclosed in any of WO 2009/151069 A1; WO 2009/037343 A1 (U.S. application Ser. No. 12/678,680); WO 2008/125487 A1 (U.S. Pat. No. 7,625,895); WO 2008/119741 A2 (U.S. application Ser. No. 12/593,721); and WO 2009/156735 A2.

In a particular embodiment, the MDM2 inhibitor is a compound of Formula I:

wherein:

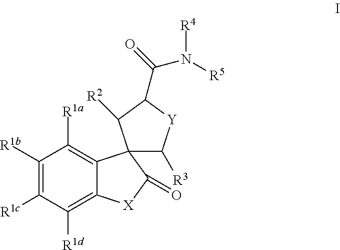

R^{1a}, R^{1b}, R^{1c}, and R^{1d} are independently selected from the group consisting of hydrogen, halogen, hydroxy, amino, nitro, cyano, alkoxy, aryloxy, optionally substituted alkyl, haloalkyl, optionally substituted cycloalkyl, optionally substituted alkenyl, optionally substituted cycloalkenyl, optionally substituted aryl, optionally substituted heteroaryl, carboxamido, and sulfonamido;

R² is selected from the group consisting of optionally substituted aryl and optionally substituted heteroaryl;

R³ is selected from the group consisting of optionally substituted alkyl, optionally substituted (cycloalkyl)alkyl, optionally substituted cycloalkyl, optionally substituted alkenyl, optionally substituted cycloalkenyl, optionally substituted aryl, and optionally substituted heteroaryl;

R⁴ is selected from the group consisting of hydrogen and optionally substituted alkyl;

R⁵ is hydrogen, optionally substituted alkyl, including but not limited to, hydroxyalkyl, dihydroxyalkyl, (cycloalkyl)alkyl, and (heterocyclo)alkyl, optionally substituted cycloalkyl; optionally substituted heterocyclo, or:

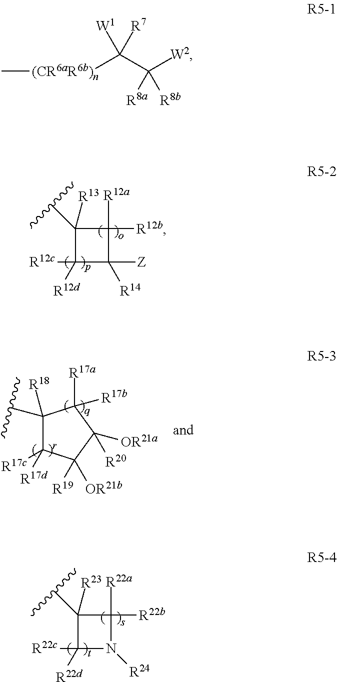

wherein:

each R^{6a} and R^{6b} is independently selected from the group consisting of hydrogen and optionally substituted C₁-C₆ alkyl;

R⁷ is selected from the group consisting of hydrogen, optionally substituted C₁-C₆ alkyl, and optionally substituted cycloalkyl;

R^{8a} and R^{8b} are each independently selected from the group consisting of hydrogen, optionally substituted C₁-C₆ alkyl, and optionally substituted cycloalkyl; or

R^{8a} and R^{8b} taken together with the carbon that they are attached form a 3- to 8-membered optionally substituted cycloalkyl;

W¹ is selected from the group consisting of —OR^{9a} and —NR^{9b}R^{9c};

R^{9a} is hydrogen;

R^{9b} is selected from the group consisting of hydrogen, optionally substituted alkyl, optionally substituted cycloalkyl, optionally substituted aryl, optionally substituted heteroaryl, —SO₂R^{9d}, and —CONR^{9e}R^{9f};

R^{9c} is selected from the group consisting of hydrogen, optionally substituted alkyl, optionally substituted cycloalkyl, optionally substituted aryl, and optionally substituted heteroaryl; or

R^{9b} and R^{9c} taken together with the nitrogen atom to which they are attached form a 4- to 8-membered optionally substituted heterocyclo;

R^{9d} is selected from the group consisting of optionally substituted alkyl and optionally substituted cycloalkyl;

R^{9e} and R^{9f} are each independently selected from the group consisting of hydrogen, optionally substituted alkyl, and optionally substituted cycloalkyl; or

R^{9e} and R^{9f} taken together with the nitrogen atom to which they are attached form a 4- to 8-membered optionally substituted heterocyclo;

W² is selected from the group consisting of —OR¹⁰ and —NR^{11a}R^{11b};

R¹⁰ is hydrogen; or

one of R^{9a} and R¹⁰ is hydrogen and the other is a metabolically cleavable group;

R^{11a} is selected from the group consisting of hydrogen, optionally substituted alkyl, optionally substituted cycloalkyl, optionally substituted aryl, optionally substituted heteroaryl, —SO₂R^{11c}, and —CONR^{11d}R^{11e};

R^{11b} is selected from the group consisting of hydrogen, optionally substituted alkyl, optionally substituted cycloalkyl, optionally substituted aryl, and optionally substituted heteroaryl; or

R^{11a} and R^{11b} taken together with the nitrogen atom to which they are attached form a 4- to 8-membered optionally substituted heterocyclo;

R^{11c} is selected from the group consisting of optionally substituted alkyl and optionally substituted cycloalkyl;

R^{11d} and R^{11e} are each independently selected from the group consisting of hydrogen, optionally substituted alkyl, and optionally substituted cycloalkyl; or

R^{11d} and R^{11e} together with the nitrogen atom to which they are attached form a 4- to 8-membered optionally substituted heterocyclo;

n is 1, 2, 3, 4, or 5;

each R^{12a}, R^{12b}, R^{12c} and R^{12d} is independently selected from the group consisting of hydrogen and optionally substituted C₁-C₆ alkyl;

R¹³ is selected from the group consisting of hydrogen and optionally substituted C₁-C₆ alkyl;

R¹⁴ is selected from the group consisting of hydrogen, optionally substituted C₁-C₆ alkyl, and optionally substituted cycloalkyl;

Z is selected from the group consisting of —OR¹⁵ and —NR^{16a}R^{16b}; or

Z and R¹⁴ taken together form a carbonyl, i.e., a C═O, group.

R¹⁵ is selected from the group consisting of hydrogen and metabolically cleavable group;

R^{16a} is selected from the group consisting of —SO₂R^{16c} and —CONR^{16d}R^{16e};

R^{16b} is selected from the group consisting of hydrogen and optionally substituted alkyl;

R^{16c} is selected from the group consisting of optionally substituted alkyl, optionally substituted cycloalkyl, optionally substituted aryl, and optionally substituted heteroaryl;

R^{16d} and R^{16e} are each independently selected from the group consisting of hydrogen, optionally substituted alkyl, optionally substituted cycloalkyl, optionally substituted aryl, and optionally substituted heteroaryl; or

R^{16d} and R^{16e} taken together with the nitrogen atom to which they are attached form a 4- to 8-membered heterocyclo;

o is 1, 2, or 3;

p is 0, 1, 2, or 3;

each R^{17a}, R^{17b}, R^{17c} and R^{17d} is independently selected from the group consisting of hydrogen and optionally substituted C₁-C₆ alkyl;

R¹⁸ is selected from the group consisting of hydrogen and optionally substituted C₁-C₆ alkyl;

R¹⁹ is selected from the group consisting of hydrogen, optionally substituted C₁-C₆ alkyl, and optionally substituted cycloalkyl;

R²⁰ is selected from the group consisting of hydrogen, optionally substituted C₁-C₆ alkyl, and optionally substituted cycloalkyl;

R^{21a} and R^{21b} are each hydrogen; or

one of R^{21a} and R^{21b} is hydrogen and the other is metabolically cleavable group;

q is 0, 1, 2, or 3;

r is 1, 2, or 3;

each R^{22a}, R^{22b}, R^{22c} and R^{22d} is independently selected from the group consisting of hydrogen and optionally substituted C₁-C₆ alkyl;

R²³ is selected from the group consisting of hydrogen and optionally substituted C₁-C₆ alkyl;

R²⁴ is selected from the group consisting of —SO₂R^{24a} and —CONR^{24b}R^{24c};

R^{24a} is selected from the group consisting of optionally substituted alkyl, optionally substituted cycloalkyl, optionally substituted aryl, and optionally substituted heteroaryl;

R^{24b} and R^{24c} are each independently selected from the group consisting of hydrogen, optionally substituted cycloalkyl, optionally substituted aryl, and optionally substituted heteroaryl; or

R^{24b} and R^{24c} taken together with the nitrogen atom to which they are attached form a 4- to 8-membered heterocyclo;

s and t are each independently 1, 2, or 3;

X is selected from the group consisting of O, S, and NR′;

Y is selected from the group consisting of O, S, and NR″;

R′ is selected from the group consisting of hydrogen, optionally substituted alkyl, aralkyl, and optionally substituted cycloalkyl; and

R″ is selected from the group consisting of hydrogen, optionally substituted alkyl, aralkyl, and optionally substituted cycloalkyl, or

R⁴ and R⁵ taken together with the nitrogen to which they are attached form a 4- to 8-membered optionally substituted heterocyclo,

or a stereoisomer thereof, or a pharmaceutically acceptable salt, solvate, or prodrug thereof.

In another particular embodiment, the MDM2 inhibitor is a compound of Formula II:

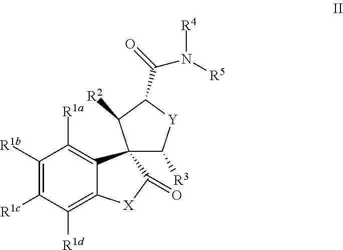

wherein R^{1a}, R^{1b}, R^{1c}, R^{1d}, R², R³, R⁴, R⁵, X, and Y have the meanings as described above for Formula I, or a pharmaceutically acceptable salt, solvate, or prodrug thereof.

In another particular embodiment, the MDM2 inhibitor is a compound of Formula II wherein:

X and Y are each NH;

R^{1a}, R^{1b}, R^{1c}, and R^{1d} are each independently selected from the group consisting of hydrogen, chloro, and fluoro;

R² is phenyl optionally substituted with chloro or fluoro;

R³ is C₁-C₆ alkyl;

R⁴ is hydrogen; and

R⁵ is selected from the group consisting of:

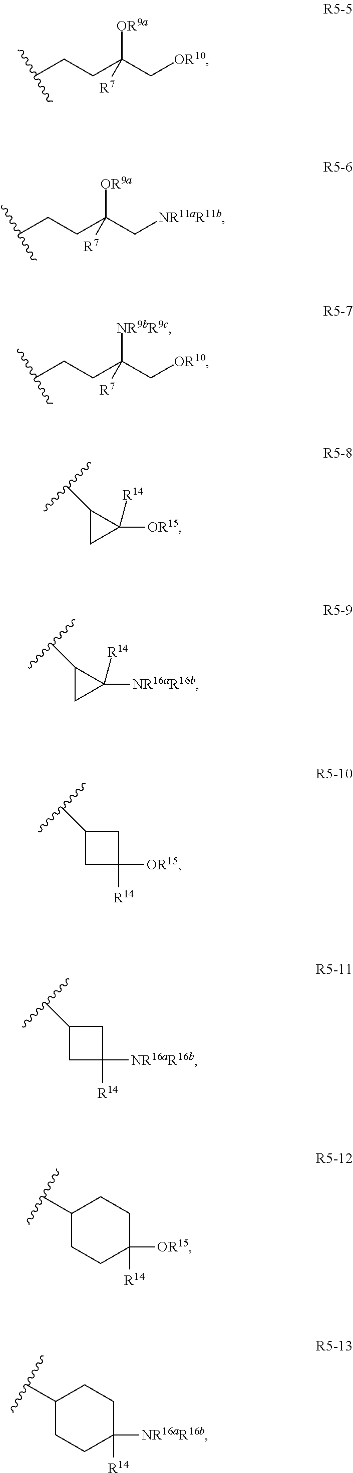

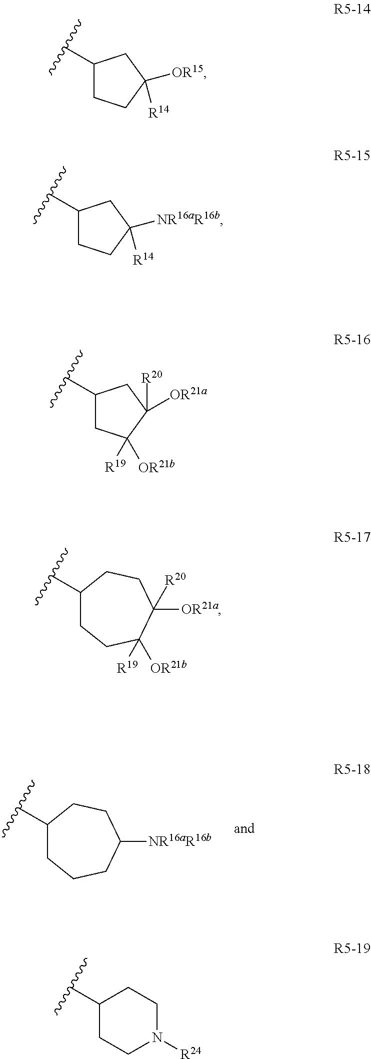

including stereoisomers, e.g., enantiomers, thereof, wherein:

R⁷ is selected from the group consisting of hydrogen and optionally substituted C₁-C₄ alkyl;

R^{9a} and R¹⁰ are each hydrogen; or

one of R^{9a} and R¹⁰ is hydrogen and the other is a metabolically cleavable group;

R^{9b} is selected from the group consisting of hydrogen, optionally substituted alkyl, optionally substituted cycloalkyl, optionally substituted aryl, optionally substituted heteroaryl, —SO₂R^{9d}, and —CONR^{9e}R^{9f};

R^{9c} is selected from the group consisting of hydrogen, optionally substituted alkyl, optionally substituted cycloalkyl, optionally substituted aryl, and optionally substituted heteroaryl; or

R^{9b} and R^{9c} taken together with the nitrogen atom to which they are attached form a 4- to 8-membered optionally substituted heterocyclo;

R^{9d} is selected from the group consisting of optionally substituted alkyl and optionally substituted cycloalkyl;

R^{9e} and R^{9f} are each independently selected from the group consisting of hydrogen, optionally substituted alkyl, and optionally substituted cycloalkyl; or

R^{9e} and R^{9f} taken together with the nitrogen atom to which they are attached form a 4- to 8-membered optionally substituted heterocyclo;

R^{11a} is selected from the group consisting of hydrogen, optionally substituted alkyl, optionally substituted cycloalkyl, optionally substituted aryl, optionally substituted heteroaryl, —SO₂R^{11c}, and —CONR^{11d}R^{11e};

R^{11b} is selected from the group consisting of hydrogen, optionally substituted alkyl, optionally substituted cycloalkyl, optionally substituted aryl, and optionally substituted heteroaryl; or

R^{11a} and R^{11b} taken together with the nitrogen atom to which they are attached form a 4- to 8-membered optionally substituted heterocyclo;

R^{11c} is selected from the group consisting of optionally substituted alkyl and optionally substituted cycloalkyl;

R^{11d} and R^{11e} are each independently selected from the group consisting of hydrogen, optionally substituted alkyl, and optionally substituted cycloalkyl; or

R^{11d} and R^{11e} taken together with the nitrogen atom to which they are attached form a 4- to 8-membered optionally substituted heterocyclo;

R¹⁴ is selected from the group consisting of hydrogen, C₁-C₄ alkyl, or C₃-C₆ cycloalkyl;

R¹⁵ is hydrogen or a metabolically cleavable group;

R^{16a} is selected from the group consisting of —SO₂R^{16c} and —CONR^{16d}R^{16e};

R^{16b} is selected from the group consisting of hydrogen and optionally substituted alkyl;

R^{16c} is selected from the group consisting of optionally substituted alkyl, optionally substituted cycloalkyl, optionally substituted aryl, and optionally substituted heteroaryl;

R^{16d} and R^{16e} are each independently selected from the group consisting of hydrogen, optionally substituted alkyl, optionally substituted cycloalkyl, optionally substituted aryl, and optionally substituted heteroaryl; or

R^{16d} and R^{16e} taken together with the nitrogen atom to which they are attached form a 4- to 8-membered heterocyclo;

R¹⁹ is selected from the group consisting of hydrogen, optionally substituted C₁-C₆ alkyl, and optionally substituted cycloalkyl;

R²⁰ is selected from the group consisting of hydrogen, optionally substituted C₁-C₆ alkyl, and optionally substituted cycloalkyl;

R^{21a} and R^{21b} are each hydrogen; or

one of R^{21a} and R^{21b} is hydrogen and the other is metabolically cleavable group;

R²⁴ is selected from the group consisting of —SO₂R^{24a} and —CONR^{24b}R^{24c};

R^{24a} is selected from the group consisting of optionally substituted alkyl, optionally substituted cycloalkyl, optionally substituted aryl, and optionally substituted heteroaryl; and

R^{24b} and R^{24a} are each independently selected from the group consisting of hydrogen, optionally substituted cycloalkyl, optionally substituted aryl, and optionally substituted heteroaryl, or

R^{24b} and R^{24c} taken together with the nitrogen atom to which they are attached form a 4- to 8-membered heterocyclo, or

or a pharmaceutically acceptable salt, solvate, or prodrug thereof.

In a particular embodiment, the MDM2 inhibitor is a compound selected from the group consisting of:

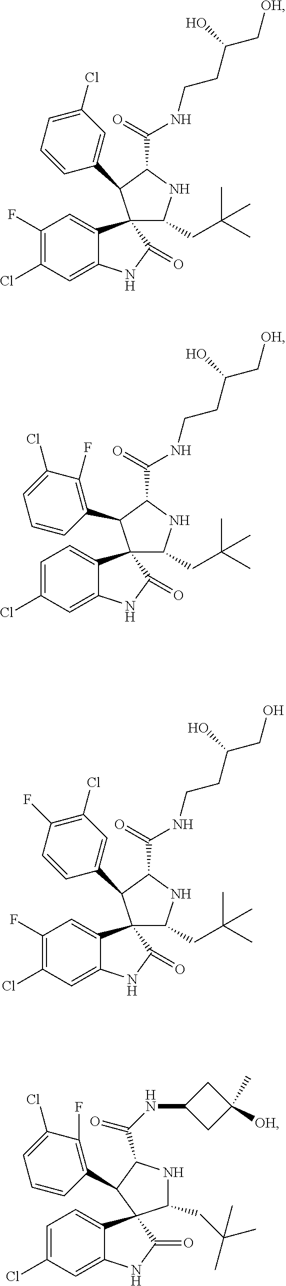

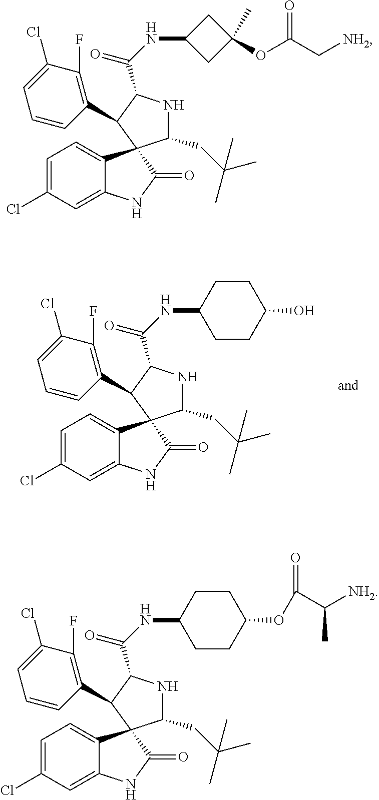

In another particular embodiment, the MDM2 inhibitor is a compound of Formula IIa:

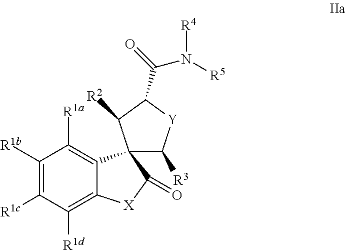

wherein R^{1a}, R_{1b}, R^{1c}, R^{1d}, R², R³, R⁴, R⁵, X, and Y have the meanings as described above for Formula I, or a pharmaceutically acceptable salt, solvate, or prodrug thereof.

In another particular embodiment, the MDM2 inhibitor is a compound of Formula IIa wherein:

R^{1a}, R^{1b} and R^{1d} are each independently selected from the group consisting of hydrogen, fluoro, and chloro;

R² is:

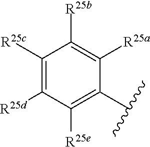

wherein:

R^{25a}, R^{25b}, R^{25c}, R^{25d}, and R^{25e} are each independently selected from the group consisting of hydrogen, fluoro, and chloro;

R³ is optionally substituted C₁-C₈ alkyl;

R⁴ is selected from the group consisting of hydrogen and optionally substituted C₁-C₆ alkyl;

R⁵ is selected from the group consisting of:

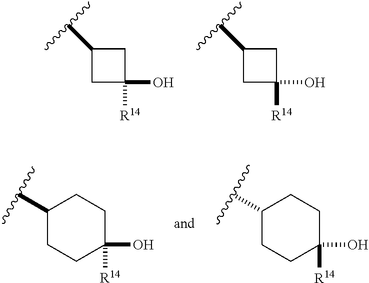

wherein:

R¹⁴ is selected from the group consisting of hydrogen and optionally substituted C₁-C₄ alkyl;

X is selected from the group consisting of O, S, and NR′;

Y is selected from the group consisting of O, S, and NR″;

R′ is selected from the group consisting of hydrogen and optionally substituted C₁-C₄ alkyl; and

R″ is selected from the group consisting of hydrogen and optionally substituted C₁-C₄ alkyl,

wherein the compound is substantially free of one or more other stereoisomers,

or a pharmaceutically acceptable salt, solvate, or prodrug thereof.

In another particular embodiment, the MDM2 inhibitor is a compound of Formula IIa wherein R⁴ is hydrogen, or a pharmaceutically acceptable salt, solvate, or prodrug thereof.

In another particular embodiment, the MDM2 inhibitor is a compound of Formula IIa wherein X is NH, or a pharmaceutically acceptable salt, solvate, or prodrug thereof.

In another particular embodiment, the MDM2 inhibitor is a compound of Formula IIa wherein Y is NH, or a pharmaceutically acceptable salt, solvate, or prodrug thereof.

In another particular embodiment, the MDM2 inhibitor is a compound of Formula IIa wherein R³ is —CH₂C(CH₃)₃, or a pharmaceutically acceptable salt, solvate, or prodrug thereof.

In another particular embodiment, the MDM2 inhibitor is a compound of Formula IIa wherein R⁵ is selected from the group consisting of:

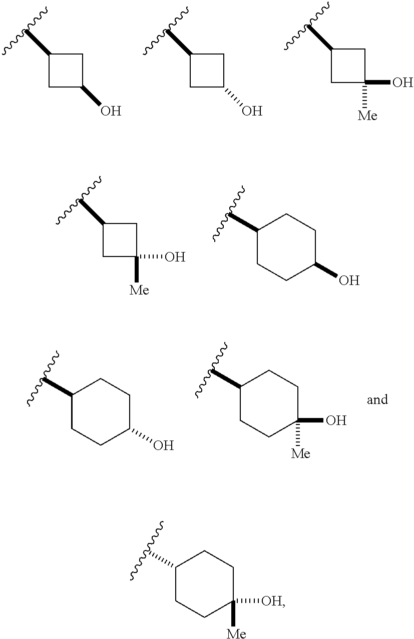

or a pharmaceutically acceptable salt, solvate, or prodrug thereof.

In another particular embodiment, the MDM2 inhibitor is a compound of Formula IIa wherein: R^{1a} is hydrogen;

R^{1a}, R^{1c}, and R^{1d} are each independently selected from the group consisting of hydrogen, fluoro, and chloro;

R³ is C₄-C₈ alkyl;

R⁴ is hydrogen;

R⁵ is selected from the group consisting of:

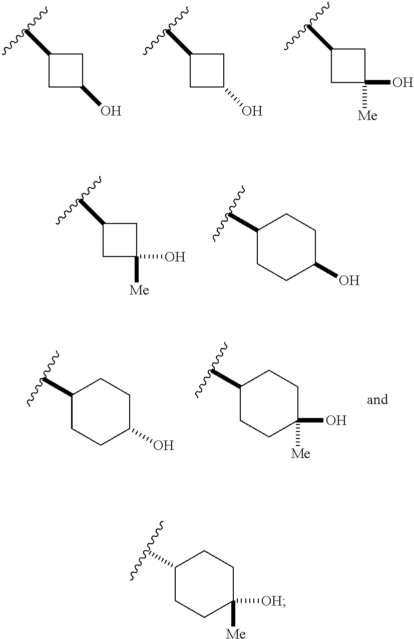

and

X and Y are NH;

or a pharmaceutically acceptable salt, solvate, or prodrug thereof.

In another particular embodiment, the MDM2 inhibitor is selected from the group consisting of:

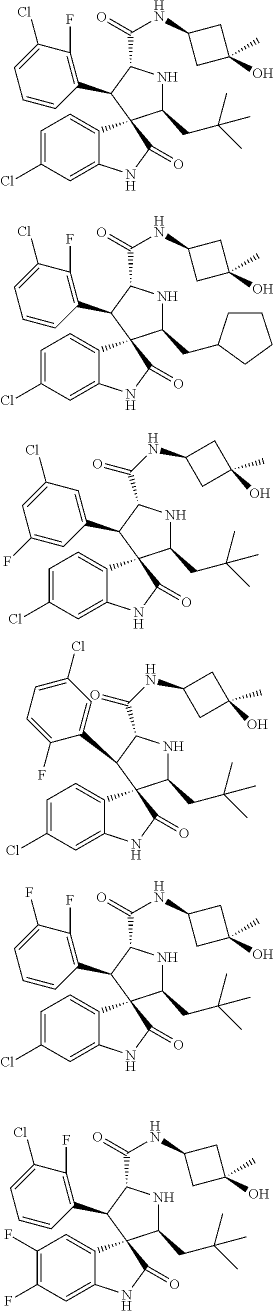

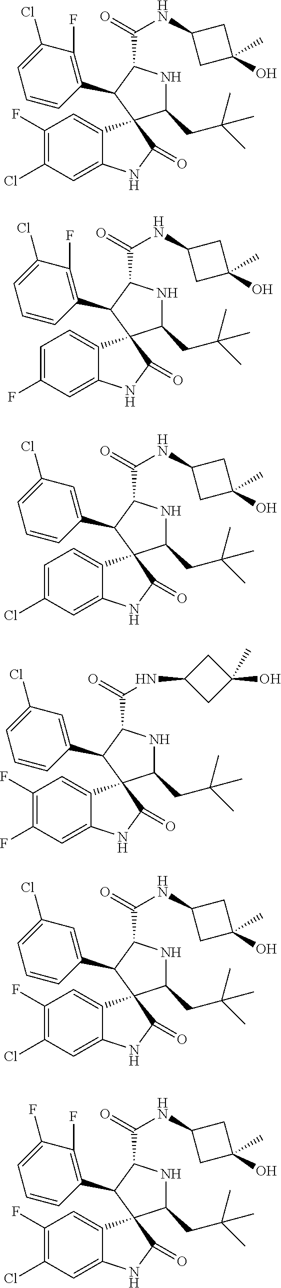

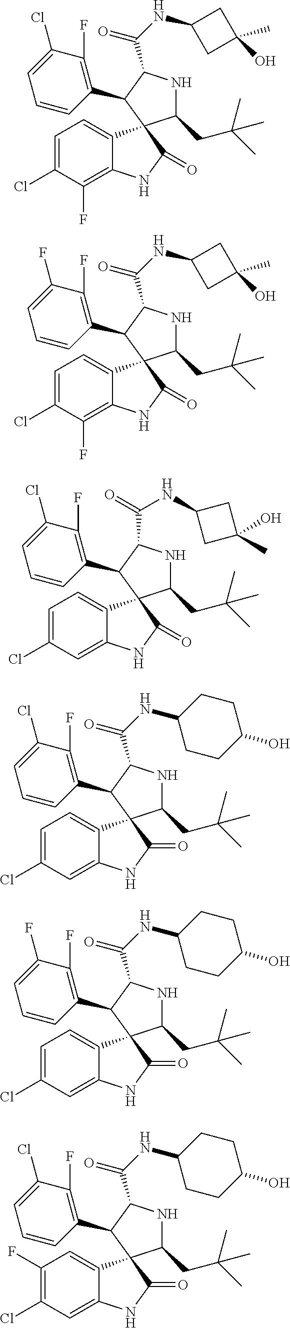

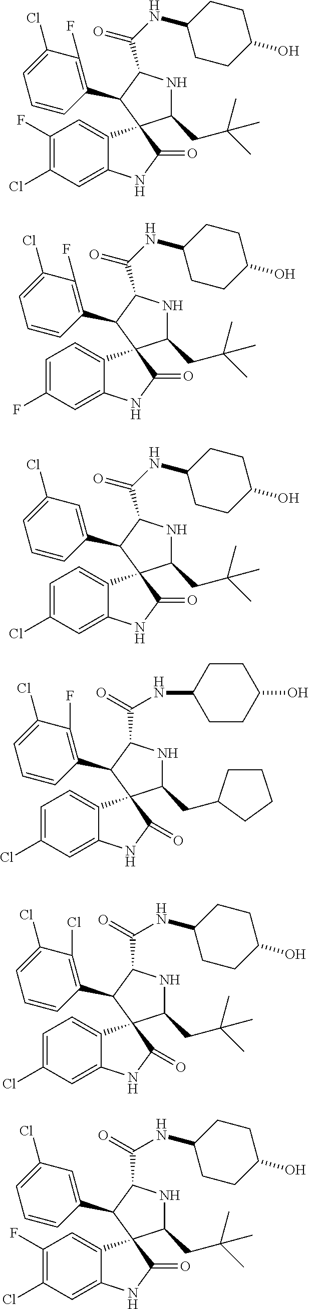

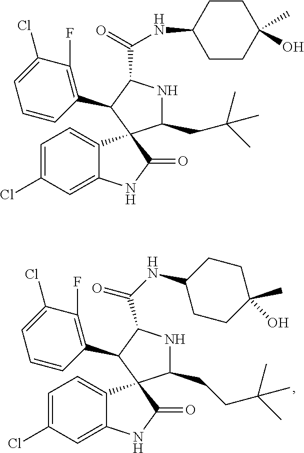

or a pharmaceutically acceptable salt, solvate, or prodrug thereof.

In another particular embodiment, the MDM2 inhibitor is:

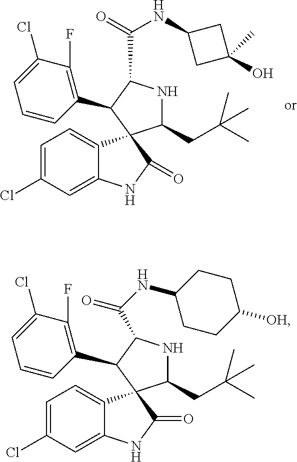

or a pharmaceutically acceptable salt, solvate, or prodrug thereof.

In a particular embodiment, the MDM2 inhibitor is any one of the inhibitors described in U.S. Pat. No. 6,734,302. For example, the MDM2 inhibitor is a compound of

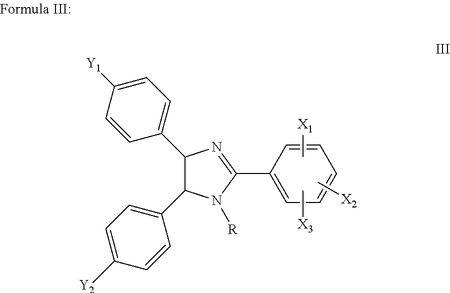

or pharmaceutically acceptable salts or esters thereof, wherein:

R is —C═OR¹;

wherein R¹ is selected from C₁-C₄ alkyl, —C═CHCOOH, —NHCH₂CH₂R², —N(CH₂CH₂OH)CH₂CH₂OH, —N(CH₃)CH₂CH₂NHCH₃, —N(CH₃)CH₂CH₂N(CH₃)CH₃, saturated 4-, 5- and 6-membered rings, and saturated and unsaturated 5- and 6-membered rings containing at least one hetero atom wherein the hetero atom is selected from S, N and O and being optionally substituted with a group selected from lower alkyl, —C═O—R⁵, —OH, lower alkyl substituted with hydroxy, lower alkyl substituted with —NH₂, N-lower alkyl, —SO₂CH₃, ═O, —CH₂C═OCH₃, and 5- and 6-membered saturated rings containing at least one hetero atom selected from S, N and O;

wherein R⁵ is selected from H, lower alkyl, —NH₂, —N-lower alkyl, lower alkyl substituted with hydroxy, and lower alkyl substituted with NH₂;

wherein R² is selected from —N(CH₃)CH₃, —NHCH₂CH₂NH₂, —NH₂, morpholinyl and piperazinyl;

X₁, X₂ and X₃ are independently selected from —OH, C₁-C₂ alkyl, C₁-C₅ alkoxy, —Cl, —Br, —F, —CH₂OCH₃, and —CH₂OCH₂CH₃;

or one of X₁, X₂ or X₃ is H and the other two are independently selected from hydroxy, lower alkyl, lower alkoxy, —Cl, —Br, —F, —CF₃, —CH₂OCH₃, —CH₂OCH₂CH₃, —OCH₂CH₂R³, —OCH₂CF₃, and —OR⁴;

or one of X₁, X₂ or X₃ is H and the other two taken together with the two carbon atoms and the bonds between them from the benzene ring to which they are substituted form a 5- or 6-membered saturated ring that contains at least one hetero atom selected from S, N, and O, wherein R³ is selected from —F, —OCH₃, —N(CH₃)CH₃, unsaturated 5- and 6-membered rings containing at least one hetero atom wherein the hetero atom is selected from S, N and O;

wherein R⁴ is a 3- to 5-membered saturated ring; and

Y₁ and Y₂ are each independently selected from —Cl, —Br, —NO₂, —C≡N, and —C≡CH.

In a particular embodiment, the MDM2 inhibitor is a compound selected from the group consisting of:

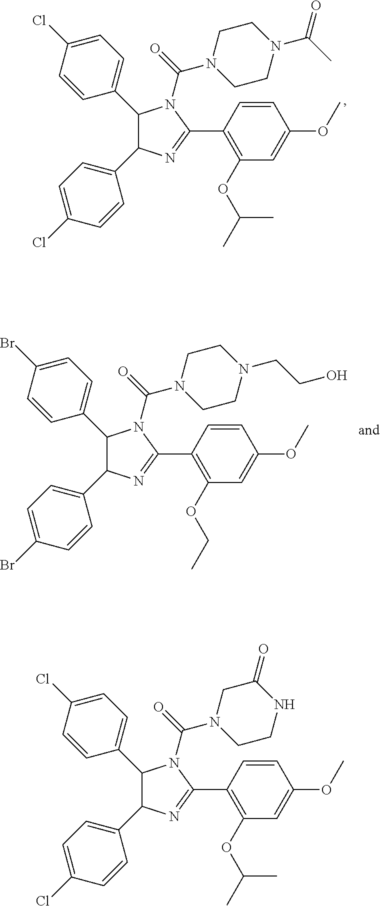

including stereoisomers, e.g., enantiomers, thereof.

In a particular embodiment, the MDM2 inhibitor is any one of the inhibitors described in WO 2009/156735 A2. For example, the MDM2 inhibitor is a compound of Formulae IV or V:

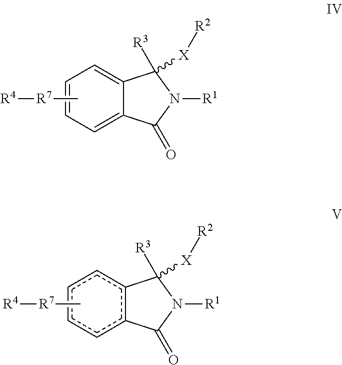

or a pharmaceutically acceptable salt thereof, wherein in both Formulae IV and V: X is selected from O, N or S;

R¹ is selected from hydrogen, substituted or unsubstituted alkyl, substituted or unsubstituted hydroxyalkyl, substituted or unsubstituted alkylamine, substituted or unsubstituted aryl, substituted or unsubstituted heteroaryl, substituted or unsubstituted aralkyl, and substituted or unsubstituted heteroaralkyl;

R² is selected from hydrogen, substituted or unsubstituted alkenyl, substituted or unsubstituted alkynyl, substituted or unsubstituted branched hydroxyalkyl, substituted or unsubstituted cycloalkyl having 6 ring carbon atoms or greater, substituted or unsubstituted cycloalkenyl, hydroxyalkylaralkyl, hydroxyalkylhetero aralkyl, and a carboxylic acid-containing group;

R³ is selected from hydrogen, substituted or unsubstituted alkyl, substituted or unsubstituted hydroxyalkyl, substituted or unsubstituted alkylamine, substituted or unsubstituted alkoxy, substituted or unsubstituted aryl, substituted or unsubstituted heteroaryl, substituted or unsubstituted aralkyl, and substituted or unsubstituted heteroaralkyl; and

R⁴-R⁷ represents groups R⁴, R⁵, R⁶ and R⁷ which are independently selected from hydrogen, halo, hydroxy, substituted or unsubstituted alkyl, substituted or unsubstituted hydroxyalkyl, substituted or unsubstituted alkenyl, substituted or unsubstituted alkynyl, substituted or unsubstituted heteroaryl, substituted or unsubstituted heteroaralkyl, substituted or unsubstituted alkylamine, substituted or unsubstituted alkoxy, trifluoromethyl, amino, nitro, carboxyl, carbonylmethylsulfone, trifluoromethylsulfone, cyano and substituted or unsubstituted sulfonamide;

wherein, where R² is substituted or unsubstituted branched hydroxyalkyl, X is O or S; and

wherein, where R² is hydrogen, at least one of R⁴-R7 is not hydrogen and R³ is not a benzimidazole derivative or a benzimidazoline derivative; and wherein, in the Formula V, the 6-membered ring may have 0, 1, or 2 C═C double bonds.

In a particular embodiment, the MDM2 inhibitor is any one of the inhibitors described in WO 2009/1511069 A1. For example, the MDM2 inhibitor is a compound of Formula VI:

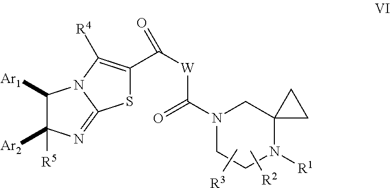

or a pharmaceutically acceptable salt thereof.

Possible examples of substituent groups include where:

Ar₁ and Ar₂ are each independently selected from the group consisting of optionally substituted aryl and optionally substituted heteroaryl;

R¹ is selected from the group consisting of hydrogen, optionally substituted alkyl, and —COR^{1a};

R^{1a} is selected from the group consisting of hydrogen, optionally substituted alkyl, optionally substituted cycloalkyl, and optionally substituted aryl;

R² and R³ are each independently selected from the group consisting of hydrogen and optionally substituted alkyl; or

R² and R³ taken together form a 3- to 6-membered optionally substituted cycloalkyl or heterocyclo;

R⁴ and R⁵ are each independently selected from the group consisting of hydrogen, optionally substituted alkyl, optionally substituted cycloalkyl, and optionally substituted aryl;

W is selected from the group consisting of:

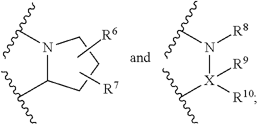

wherein:

R⁶ and R⁷ are each independently selected from the group consisting of hydrogen, hydroxy and optionally substituted alkyl; or

R⁶ and R⁷ taken together form a 3- to 6-membered optionally substituted cycloalkyl or an oxo, i.e., C═O;

R⁸ is selected from the group consisting of hydrogen or optionally substituted alkyl;

R⁹ and R¹⁰ are each independently selected from the group consisting of hydrogen or optionally substituted alkyl; or

R⁹ and R¹⁰ taken together form a 3- to 6-membered optionally substituted cycloalkyl or heterocyclo; and

X is a carbon atom.

In a particular embodiment, MDM2 inhibitor is a compound of Formula VI wherein possible examples of substituent groups include where:

Ar₁ and Ar₂ are each independently selected from the group consisting of optionally substituted phenyl and optionally substituted pyridyl;

R¹ is selected from the group consisting of hydrogen, optionally substituted C₁-C₆ alkyl, and —COR^{1a};

R^{1a} is selected from the group consisting of hydrogen and optionally substituted C₁-C₆ alkyl;

R² and R³ are each independently selected from the group consisting of hydrogen and optionally substituted C₁-C₆ alkyl; or

R² and R³ taken together form a 3- to 6-membered optionally substituted cycloalkyl;

R⁴ and R⁵ are each independently selected from the group consisting of hydrogen and optionally substituted C₁-C₆ alkyl;

W is:

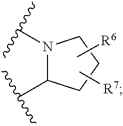

wherein:

R⁶ and R⁷ are each independently selected from the group consisting of hydrogen and optionally substituted C₁-C₆ alkyl; or

R⁶ and R⁷ taken together form a 3- to 6-membered optionally substituted cycloalkyl or an oxo.

The term “metabolically cleavable group” as used herein, refers to groups which can be cleaved from the parent molecule by metabolic processes and be substituted with hydrogen. Certain compounds containing metabolically cleavable groups may be prodrugs, i.e., they are pharmacologically inactive. Certain other compounds containing metabolically cleavable groups may be antagonists of the interaction between p53 and MDM2. In such cases, these compounds may have more, less, or equivalent activity of the parent molecule. Examples of metabolically cleavable groups include those derived from amino acids (see, e.g., US 2006/0241017 Al; US 2006/0287244 A1; and WO 2005/046575 A2) or phosphorus-containing compounds (see, e.g., U.S. 2007/0249564 A1) as illustrated in Scheme 1.

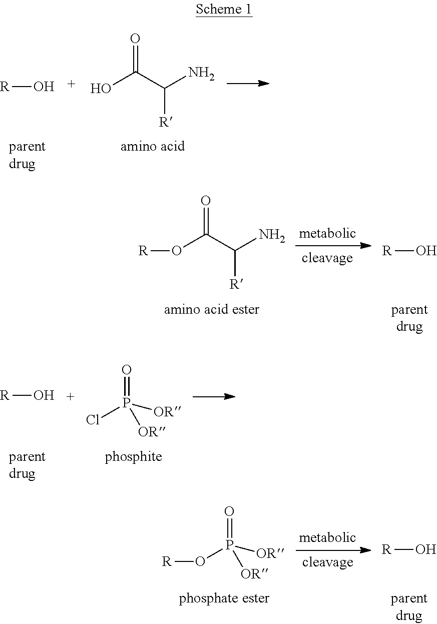

The term “pharmaceutically acceptable salt” as used herein, refers to any salt (e.g., obtained by reaction with an acid or a base) of a compound provided herein that is physiologically tolerated in the target animal (e.g., a mammal). Salts of the compounds of provided herein may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds provided herein including pharmaceutically acceptable acid addition salts thereof.

Examples of bases include, but are not limited to, alkali metal (e.g., sodium) hydroxides, alkaline earth metal (e.g., magnesium) hydroxides, ammonia, and compounds of formula NW₄³⁰ , wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, mesylate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of provided herein compounded with a suitable cation such as Na⁺, NH₄, and NW₄^{|} (wherein W is a C₁₋₄ alkyl group), and the like. For therapeutic use, salts of the compounds provided herein are contemplated as being pharmaceutically acceptable. However, salts of acids and bases that are non-pharmaceutically acceptable may also find use, for example, in the preparation or purification of a pharmaceutically acceptable compound.

The term “solvate” as used herein, refers to the physical association of a compound provided herein with one or more solvent molecules, whether organic or inorganic. This physical association often includes hydrogen bonding. In certain instances, the solvate is capable of isolation, for example, when one or more solvate molecules are incorporated in the crystal lattice of the crystalline solid. “Solvate” encompasses both solution-phase and isolable solvates. Exemplary solvates include hydrates, ethanolates, and methanolates.

The term “monovalent pharmaceutically acceptable cation” as used herein refers to inorganic cations such as, but not limited to, alkaline metal ions, e.g., Na⁺ and K⁺, as well as organic cations such as, but not limited to, ammonium and substituted ammonium ions, e.g., NH₄⁺, NHMe₃⁺, NH₂Me₂⁺, NHMe₃⁺ and NMe₄⁺.

The term “divalent pharmaceutically acceptable cation” as used herein refers to inorganic cations such as, but not limited to, alkaline earth metal cations, e.g., Ca²⁺ and Mg²⁺.

Examples of monovalent and divalent pharmaceutically acceptable cations are discussed, e.g., in Berge et al. J. Pharm. Sci., 66:1-19 (1997).

The term “therapeutically effective amount,” as used herein, refers to that amount of the therapeutic agent sufficient to result in amelioration of one or more symptoms of a disorder, or prevent advancement of a disorder, or cause regression of the disorder. For example, with respect to the treatment of cancer, e.g., leukemia, in one embodiment, a therapeutically effective amount will refer to the amount of a therapeutic agent that causes a therapeutic response, e.g., normalization of blood counts, decrease in the rate of tumor growth, decrease in tumor mass, decrease in the number of metastases, increase in time to tumor progression, and/or increase in survival time by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%, or more.

The term “pharmaceutically acceptable carrier” or “pharmaceutically acceptable vehicle” encompasses any of the standard pharmaceutical carriers, solvents, surfactants, or vehicles. Suitable pharmaceutically acceptable vehicles include aqueous vehicles and nonaqueous vehicles. Standard pharmaceutical carriers and their formulations are described in Remington's Pharmaceutical Sciences, Mack Publishing Co., Easton, Pa., 19th ed. 1995.

The term “alkyl” as used herein by itself or part of another group refers to a straight-chain or branched saturated aliphatic hydrocarbon having from one to eighteen carbons or the number of carbons designated (e.g., C₁-C₁₈ means 1 to 18 carbons). In one embodiment, the alkyl is a C₁-C₁₀ alkyl. In another embodiment, the alkyl is a C₁-C₆ alkyl. In another embodiment, the alkyl is a C₁-C₄ alkyl. Exemplary alkyl groups include methyl, ethyl, n-propyl, isopropyl, n-butyl, sec-butyl, isobutyl, tent-butyl, n-pentyl, n-hexyl, isohexyl, n-heptyl, 4,4-dimethylpentyl, n-octyl, 2,2,4-trimethylpentyl, nonyl, decyl and the like.

The term “optionally substituted alkyl” as used herein by itself or part of another group means that the alkyl as defined above is either unsubstituted or substituted with one, two or three substituents independently selected from hydroxy (i.e., —OH), nitro (i.e., —NO₂), cyano (i.e., —CN), optionally substituted cycloalkyl, optionally substituted heteroaryl, optionally substituted heterocyclo, alkoxy, aryloxy, aralkyloxy, alkylthio, carboxamido or sulfonamido. In one embodiment, the optionally substituted alkyl is substituted with two substituents. In another embodiment, the optionally substituted alkyl is substituted with one substituent. In another embodiment, the substituents are selected from hydroxyl (i.e., a hydroxyalkyl), optionally substituted cycloalkyl (i.e., a (cycloalkyl)alkyl), or amino (i.e., an aminoalkyl). Exemplary optionally substituted alkyl groups include —CH₂OCH₃, —CH₂CH₂NH₂, —CH₂CH₂NH(CH₃), —CH₂CH₂CN, —CH₂SO₂CH₃, hydroxymethyl, hydroxyethyl, hydroxypropyl, and the like.

The term “alkylenyl” as used herein by itself or part of another group refers to a divalent alkyl radical containing one, two, three, four, or more joined methylene groups. Exemplary alkylenyl groups include —(CH₂)—, —(CH₂)₂—, —(CH₂)₃—, —(CH₂)₄—, and the like.

The term “optionally substituted alkylenyl” as used herein by itself or part of another group means the alkylenyl as defined above is either unsubstituted or substituted with one, two, three, or four substituents independently selected from the group consisting of optionally substituted C₁-C₆ alkyl, optionally substituted cycloalkyl, optionally substituted aryl, and optionally substituted heteroaryl. In one embodiment, the optionally substituted C₁-C₆ alkyl is methyl. In one embodiment, the optionally substituted aryl is a phenyl optionally substituted with one or two halo groups. Exemplary optionally substituted alkylenyl groups include —CH(CH₃)—, —C(CH₃)₂—, —CH₂CH(CH₃)—, —CH₂CH(CH₃)CH₂—, —CH₂CH(Ph)CH₂—, —CH(CH₃)CH(CH₃)—, and the like.

The term “haloalkyl” as used herein by itself or part of another group refers to an alkyl as defined above having one to six halo substituents. In one embodiment, the haloalkyl has one, two or three halo substituents. Exemplary haloalkyl groups include trifluoromethyl, —CH₂CH₂F and the like.

The term “hydroxyalkyl” as used herein by itself or part of another group refers to an alkyl as defined above having one hydroxy substituent. Exemplary hydroxyalkyl groups include hydroxymethyl, hydroxyethyl, hydroxypropyl, and the like.

The term “dihydroxyalkyl” as used herein by itself or part of another group refers to alkyl as defined above having two hydroxyl substituents. Exemplary dihydroxyalkyl groups include —CH₂CH₂CCH₃(OH)CH₂OH, —CH₂CH₂CH(OH)CH(CH₃)OH, —CH₂CH(CH₂OH)₂, —CH₂CH₂CH(OH)C(CH₃)₂OH—CH—₂CH₂CCH₃(OH)CH(CH₃)OH, and the like, including stereoisomers thereof.

The term “hydroxycycloalkyl” as used herein by itself or part of another group refers to an optionally substituted cycloalkyl as defined below having a least one, e.g., one or two hydroxy substituents. Exemplary hydroxycycloalkyl groups include:

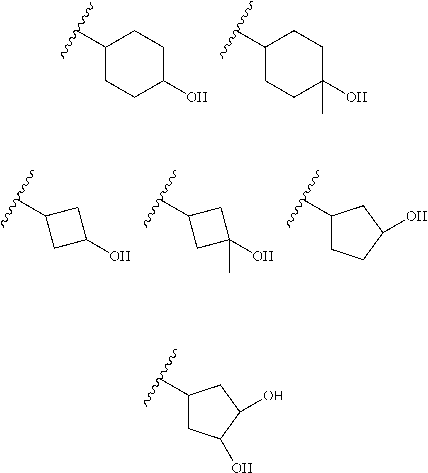

and the like, including stereoisomers thereof.

The term “optionally substituted (cycloalkyl)alkyl” as used herein by itself or part of another group refers to an optionally substituted alkyl as defined above having an optionally substituted cycloalkyl (as defined below) substituent. Exemplary optionally substituted (cycloalkyl)alkyl groups include:

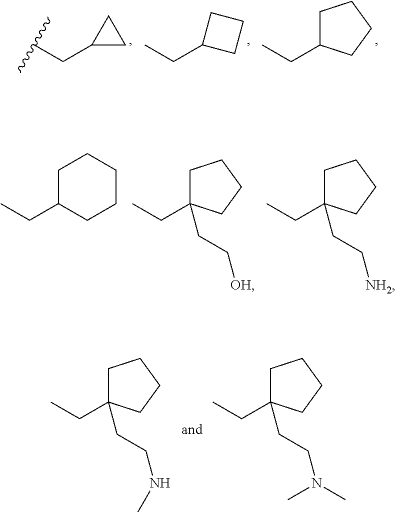

and the like, including stereoisomers thereof.

The term “aralkyl” as used herein by itself or part of another group refers to an optionally substituted alkyl as defined above having one, two or three optionally substituted aryl substituents. In one embodiment, the aralkyl has two optionally substituted aryl substituents. In another embodiment, the aralkyl has one optionally substituted aryl substituent. In another embodiment, the aralkyl is an aryl(C₁-C₄ alkyl). In another embodiment, the aryl(C₁-C₄ alkyl) has two optionally substituted aryl substituents. In another embodiment, the aryl(C₁-C₄ alkyl) has one optionally substituted aryl substituent. Exemplary aralkyl groups include, for example, benzyl, phenylethyl, (4-fluorophenyl)ethyl, phenylpropyl, diphenylmethyl (i.e., Ph₂CH—), diphenylethyl (Ph₂CHCH₂—) and the like.

The term “cycloalkyl” as used herein by itself or part of another group refers to saturated and partially unsaturated (containing one or two double bonds) cyclic hydrocarbon groups containing one to three rings having from three to twelve carbon atoms (i.e., C₃-C₁₂ cycloalkyl) or the number of carbons designated. In one embodiment, the cycloalkyl has one ring. In another embodiment, the cycloalkyl is a C₃-C₆ cycloalkyl. Exemplary cycloalkyl groups include cyclopropyl, cyclobutyl, cyclopentyl, cyclohexyl, cycloheptyl, cyclooctyl, norbornyl, decalin, adamantyl and the like.

The term “optionally substituted cycloalkyl” as used herein by itself or part of another group means the cycloalkyl as defined above is either unsubstituted or substituted with one, two or three substituents independently selected from halo, nitro, cyano, hydroxy, amino, optionally substituted alkyl, haloalkyl, hydroxyalkyl, aminoalkyl, aralkyl, optionally substituted cycloalkyl, optionally substituted alkenyl, optionally substituted alkynyl, optionally substituted aryl, optionally substituted heteroaryl, optionally substituted heterocyclo, alkoxy, aryloxy, aralkyloxy, alkylthio, carboxamido or sulfonamido. The term “optionally substituted cycloalkyl” also means the cycloalkyl as defined above may be fused to an optionally substituted aryl. Exemplary optionally substituted cycloalkyl groups include

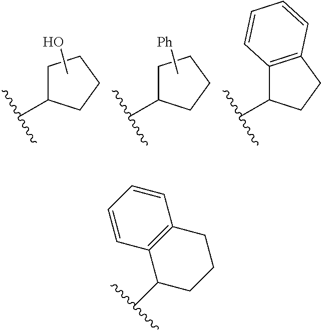

and the like.

The term “alkenyl” as used herein by itself or part of another group refers to an alkyl group as defined above containing one, two or three carbon-to-carbon double bonds. In one embodiment, the alkenyl has one carbon-to-carbon double bond. Exemplary alkenyl groups include —CH═CH₂, —CH₂CH═CH₂, —CH₂CH₂CH═CH₂, —CH₂CH₂CH═CHCH₃ and the like.

The term “optionally substituted alkenyl” as used herein by itself or part of another group means the alkenyl as defined above is either unsubstituted or substituted with one, two or three substituents independently selected from halo, nitro, cyano, hydroxy, amino, optionally substituted alkyl, haloalkyl, hydroxyalkyl, aralkyl, optionally substituted cycloalkyl, optionally substituted alkenyl, optionally substituted alkynyl, optionally substituted aryl, optionally substituted heteroaryl, optionally substituted heterocyclo, alkoxy, aryloxy, aralkyloxy, alkylthio, carboxamido or sulfonamido. Exemplary optionally substituted alkenyl groups include —CH═CHPh, —CH₂CH═CHPh and the like.

The term “cycloalkenyl” as used herein by itself or part of another group refers to a cycloalkyl group as defined above containing one, two or three carbon-to-carbon double bonds. In one embodiment, the cycloalkenyl has one carbon-to-carbon double bond. Exemplary cycloalkenyl groups include cyclopentene, cyclohexene and the like.

The term “optionally substituted cycloalkenyl” as used herein by itself or part of another group means the cycloalkenyl as defined above is either unsubstituted or substituted with one, two or three substituents independently selected from halo, nitro, cyano, hydroxy, amino, optionally substituted alkyl, haloalkyl, hydroxyalkyl, aralkyl, optionally substituted cycloalkyl, optionally substituted alkenyl, optionally substituted alkynyl, optionally substituted aryl, optionally substituted heteroaryl, optionally substituted heterocyclo, alkoxy, aryloxy, aralkyloxy, alkylthio, carboxamido or sulfonamido.

The term “alkynyl” as used herein by itself or part of another group refers to an alkyl group as defined above containing one to three carbon-to-carbon triple bonds. In one embodiment, the alkynyl has one carbon-to-carbon triple bond. Exemplary alkynyl groups include —C≡CH, —C≡CCH₃, —CH₂C≡CH, —CH₂CH₂C≡CH and —CH₂CH₂C≡CCH₃.

The term “optionally substituted alkynyl” as used herein by itself or part of another group means the alkynyl as defined above is either unsubstituted or substituted with one, two or three substituents independently selected from halo, nitro, cyano, hydroxy, amino, optionally substituted alkyl, haloalkyl, hydroxyalkyl, aralkyl, optionally substituted cycloalkyl, optionally substituted alkenyl, optionally substituted alkynyl, optionally substituted aryl, optionally substituted heteroaryl, optionally substituted heterocyclo, alkoxy, aryloxy, aralkyloxy, alkylthio, carboxamido or sulfonamido. Exemplary optionally substituted alkenyl groups include —C≡CPh, —CH₂C≡CPh and the like.

The term “aryl” as used herein by itself or part of another group refers to monocyclic and bicyclic aromatic ring systems having from six to fourteen carbon atoms (i.e., C₆-C₁₄ aryl) such as phenyl (abbreviated as Ph), 1-naphthyl and 2-naphthyl and the like.

The term “optionally substituted aryl” as used herein by itself or part of another group means the aryl as defined above is either unsubstituted or substituted with one to five substituents independently selected from halo, nitro, cyano, hydroxy, amino, optionally substituted alkyl, haloalkyl, hydroxyalkyl, aralkyl, optionally substituted cycloalkyl, optionally substituted alkenyl, optionally substituted alkynyl, optionally substituted aryl, optionally substituted heteroaryl, optionally substituted heterocyclo, alkoxy, aryloxy, aralkyloxy, alkylthio, carboxamido or sulfonamido. In one embodiment, the optionally substituted aryl is an optionally substituted phenyl. In one embodiment, the optionally substituted phenyl has four substituents. In another embodiment, the optionally substituted phenyl has three substituents. In another embodiment, the optionally substituted phenyl has two substituents. In another embodiment, the optionally substituted phenyl has one substituent. Exemplary substituted aryl groups include 2-methylphenyl, 2-methoxyphenyl, 2-fluorophenyl, 2-chlorophenyl, 2-bromophenyl, 3-methylphenyl, 3-methoxyphenyl, 3-fluorophenyl, 3-chlorophenyl, 4-methylphenyl, 4-ethylphenyl, 4-methoxyphenyl, 4-fluorophenyl, 4-chlorophenyl, 2,6-di-fluorophenyl, 2,6-di-chlorophenyl, 2-methyl, 3-methoxyphenyl, 2-ethyl, 3-methoxyphenyl, 3,4-di-methoxyphenyl, 3,5-di-fluorophenyl 3,5-di-methylphenyl and 3,5-dimethoxy, 4-methylphenyl, 2-fluoro-3-chlorophenyl, 3-chloro-4-fluorophenyl and the like. The term optionally substituted aryl is meant to include groups having fused optionally substituted cycloalkyl and fused optionally substituted heterocyclo rings. Examples include

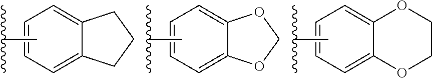

and the like.

The term “heteroaryl” as used herein by itself or part of another group refers to monocyclic and bicyclic aromatic ring systems having from five to fourteen carbon atoms (i.e., C₅-C₁₄ heteroaryl) and one, two, three or four heteroatoms independently selected from the group consisting of oxygen, nitrogen and sulfur. In one embodiment, the heteroaryl has three heteroatoms. In one embodiment, the heteroaryl has two heteroatoms. In one embodiment, the heteroaryl has one heteroatom. Exemplary heteroaryl groups include 1-pyrrolyl, 2-pyrrolyl, 3-pyrrolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, purinyl, 2-benzimidazolyl, 4-benzimidazolyl, 5-benzimidazolyl, 2-benzthiazolyl, 4-benzthiazolyl, 5-benzthiazolyl, 5-indolyl, 3-indazolyl, 4-indazolyl, 5-indazolyl, 1-isoquinolyl, 5-isoquinolyl, 2-quinoxalinyl, 5-quinoxalinyl, 2-quinolyl 3-quinolyl, 6-quinolyl and the like. The term heteroaryl is meant to include possible N-oxides. Exemplary N-oxides include pyridyl N-oxide and the like.

The term “optionally substituted heteroaryl” as used herein by itself or part of another group means the heteroaryl as defined above is either unsubstituted or substituted with one to four substituents, typically one or two substituents, independently selected from halo, nitro, cyano, hydroxy, amino, optionally substituted alkyl, haloalkyl, hydroxyalkyl, aralkyl, optionally substituted cycloalkyl, optionally substituted alkenyl, optionally substituted alkynyl, optionally substituted aryl, optionally substituted heteroaryl, optionally substituted heterocyclo, alkoxy, aryloxy, aralkyloxy, alkylthio, carboxamido or sulfonamido. In one embodiment, the optionally substituted heteroaryl has one substituent. In another embodiment, the substituent is an optionally substituted aryl, aralkyl, or optionally substituted alkyl. In another embodiment, the substituent is an optionally substituted phenyl. Any available carbon or nitrogen atom may be substituted. Exemplary optionally substituted heteroaryl groups include

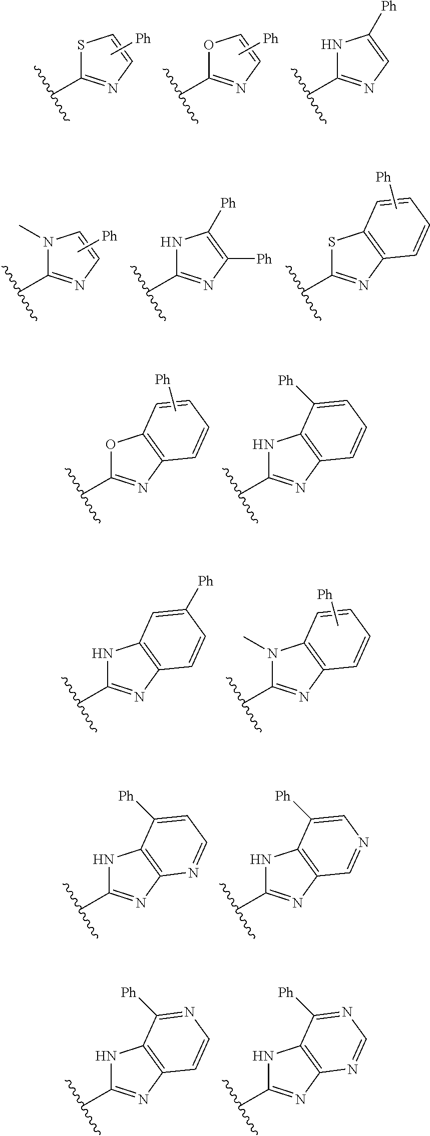

and the like.

The term “heterocyclo” as used herein by itself or part of another group refers to saturated and partially unsaturated (containing one or two double bonds) cyclic groups containing one to three rings having from two to twelve carbon atoms (i.e., C₂-C₁₂ heterocyclo) and one or two oxygen, sulfur or nitrogen atoms. The heterocyclo can be optionally linked to the rest of the molecule through a carbon or nitrogen atom. Exemplary heterocyclo groups include

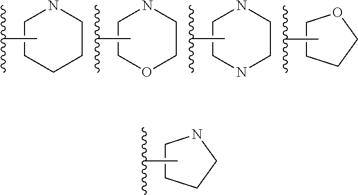

and the like.

The term “optionally substituted heterocyclo” as used herein by itself or part of another group means the heterocyclo as defined above is either unsubstituted or substituted with one to four substituents independently selected from halo, nitro, cyano, hydroxy, amino, optionally substituted alkyl, haloalkyl, hydroxyalkyl, aralkyl, optionally substituted cycloalkyl, optionally substituted alkenyl, optionally substituted alkynyl, optionally substituted aryl, optionally substituted heteroaryl, optionally substituted heterocyclo, alkoxy, aryloxy, aralkyloxy, alkylthio, carboxamido, sulfonamido, —COR^{c}, —SO₂R^{d}, —N(R^{e})COR^{f}, —N(R^{e})SO₂R^{g} or —N(R^{e})C═N(R^{h})-amino, wherein R^{c} is hydrogen, optionally substituted alkyl, optionally substituted aryl, or optionally substituted heteroaryl; R^{d} is optionally substituted alkyl, optionally substituted aryl, or optionally substituted heteroaryl; R^{e} is hydrogen, optionally substituted alkyl, optionally substituted aryl, or optionally substituted heteroaryl; R^{f} is hydrogen, optionally substituted alkyl, optionally substituted aryl, or optionally substituted heteroaryl; R^{g} is optionally substituted alkyl, optionally substituted aryl, or optionally substituted heteroaryl; and R^{h} is hydrogen, —CN, optionally substituted alkyl, optionally substituted aryl, or optionally substituted heteroaryl. Substitution may occur on any available carbon or nitrogen atom. Exemplary substituted heterocyclo groups include

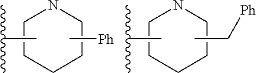

and the like. An optionally substituted heterocyclo may be fused to an aryl group to provide an optionally substituted aryl as described above.

The term “alkoxy” as used herein by itself or part of another group refers to a haloalkyl, optionally substituted alkyl, optionally substituted cycloalkyl, optionally substituted alkenyl or optionally substituted alkynyl attached to a terminal oxygen atom. Exemplary alkoxy groups include methoxy, tert-butoxy, —OCH₂CH═CH₂ and the like.

The term “aryloxy” as used herein by itself or part of another group refers to an optionally substituted aryl attached to a terminal oxygen atom. Exemplary aryloxy groups include phenoxy and the like.

The term “aralkyloxy” as used herein by itself or part of another group refers to an aralkyl attached to a terminal oxygen atom. Exemplary aralkyloxy groups include benzyloxy and the like.

The term “alkylthio” as used herein by itself or part of another group refers to a haloalkyl, aralkyl, optionally substituted alkyl, optionally substituted cycloalkyl, optionally substituted alkenyl or optionally substituted alkynyl attached to a terminal sulfur atom. Exemplary alkyl groups include —SCH₃ and the like.

The term “halo” or “halogen” as used herein by itself or part of another group refers to fluoro, chloro, bromo or iodo. In one embodiment, the halo is fluoro or chloro.

The term “amino” as used herein by itself or part of another group refers to a radical of formula —NR^{a}R^{b} wherein R^{a} and R^{b} are independently hydrogen, haloalkyl, aralkyl, optionally substituted alkyl, optionally substituted cycloalkyl, optionally substituted heterocyclo, optionally substituted aryl or optionally substituted heteroaryl; or R^{a} and R^{b} taken together with the nitrogen atom to which they are attached form a four to seven membered optionally substituted heterocyclo. Exemplary amino groups include —NH₂, —N(H)CH₃, —N(CH₃)₂, N(H)CH₂CH₃, N(CH₂CH₃), —N(H)CH₂Ph and the like.

The term “carboxamido” as used herein by itself or part of another group refers to a radical of formula —CO-amino. Exemplary carboxamido groups include —CONH₂, —CON(H)CH₃, —CON(H)Ph, —CON(H)CH₂CH₂Ph, —CON(CH₃)₂, CON(H)CHPh₂ and the like.

The term “sulfonamido” as used herein by itself or part of another group refers to a radical of formula —SO₂-amino. Exemplary sulfonamido groups include —SO₂NH₂, —SO₂N(H)CH₃, —SO₂N(H)Ph and the like.

The term “about,” as used herein, includes the recited number±10%. Thus, “about 10” means 9 to 11.

Certain MDM2 inhibitors may exist as stereoisomers including optical isomers. The methods and compositions provided herein includes or includes the use of all stereoisomers, both as pure individual stereoisomer preparations and enriched preparations of each, and both the racemic mixtures of such stereoisomers as well as the individual diastereomers and enantiomers that may be separated according to methods that are well known to those of skill in the art.

The term “substantially free of as used herein means that the compound comprises less than about 25% of other stereoisomers, e.g., diastereomers and/or enantiomers, as established using conventional analytical methods routinely used by those of skill in the art. In some embodiments, the amount of other stereoisomers is less than about 24%, less than about 23%, less than about 22%, less than about 21%, less than about 20%, less than about 19%, less than about 18%, less than about 17%, less than about 16%, less than about 15%, less than about 14%, less than about 13%, less than about 12%, less than about 11%, less than about 10%, less than about 9%, less than about 8%, less than about 7%, less than about 6%, less than about 5%, less than about 4%, less than about 3%, less than about 2%, less than about 1%, or less than about 0.5%.

Methods for determining whether the cells of a subject contain an activating mutation of FLT3 and thus test positive for such a mutation, are well known to those of ordinary skill in the art. For example, see Kiyoi et al., U.S. Pat. No. 7,125,659; Kottaridis, P. D. et al., Blood 98: 1752 (2010); Sawyers, C. L., Cold Spring Harbor Symposia On Quantitative Biology LXX: 479 (2005); and Vande Woude, G. F. et al., Clinical Cancer Research 10: 3897 (2004).

Methods for determining whether the cells of a subject contain at least one mutation in the p53 gene, and, thus test positive for such a mutation(s), are also well known to those of ordinary skill in the art. For example, seek Flaman, J.-M., et al., Proc. Natl. Acad. Sci. USA 92: 3963-3967 (1995). In one embodiment, the mutation(s) are detected by direct sequencing of the gene. In another embodiment, the mutation(s) are detected by PCR.

The party that determines whether the subject's cells contain FLT3 having an activating mutation such as FLT3-ITD or a p53 gene mutation may or may not be the same party that selects the subject for treatment for leukemia. In one embodiment, a single party determines whether the subject's cells contain the FLT3 or p53 gene mutation and selects the subject for treatment for leukemia. In another embodiment, one party, e.g., an analytical assay service, determines whether the subject's cells contain a FLT3-ITD or p53 gene mutation, and another party, e.g., a physician or health care professional, selects the subject for treatment for leukemia, e.g., by reviewing the results provided by an analytical assay service.

The term “anticancer agent” as used herein, refers to any therapeutic agent (e.g., chemotherapeutic compound and/or molecular therapeutic compound), antisense therapy, radiation therapy, or surgical intervention, used in the treatment of cancer (e.g., in mammals, e.g., in humans). Anticancer agents for the treatment of leukemia include, but are not limited to, fludarabine phosphate, cladribine, clofarbine laromustine, and ara-C (Grant, S., Best Pract. Res. Clin. Haematol. 22:501-507 (2009)). Anticancer agents are well known to those of ordinary skill in the art (See any number of instructive manuals including, but not limited to, the Physician's Desk Reference and Goodman and Gilman's “Pharmaceutical Basis of Therapeutics” tenth edition, Eds. Hardman et al., 2002).

In one embodiment, the leukemia is treated by administering an MDM2 inhibitor and at least one other anticancer agent. In one embodiment, the other anticancer agent is an FLT3 inhibitor. In another embodiment, the FLT3 inhibitor is FI-700. In another embodiment, the FLT3 inhibitor is semaxinib, sunitinib (SU11248), lestaurtinib (CEP-701), midostaurin (PKC412), sorafenib, tandutinib, KW-2449, AC220, AG1295, AG1296, AGL2043, D64406, SU5416, SU5614, MLN518, GTP-14564, Ki23819, and CHIR-258 (See, for example, Small, D., Hematology Am. Soc. Hematol. Educ. Program 178-84 (2006) and Grant, S., Best Pract. Res. Clin. Haematol. 22:501-507 (2009)).

Detailed characterization of sensitivity and resistance to treatment ex vivo with the MDM2 inhibitor MI-219 in AML blasts from 109 patients is provided herein. In line with previous observations, all AML cases with mutated p.53 were resistant to MI-219. Importantly, ˜30% of AML cases with unmutated p.53 also demonstrated primary resistance to MI-219. Analysis of potential mechanisms associated with MI-219 resistance in AML blasts with wild type p53 uncovered distinct molecular defects including low or absent p53 protein induction following MDM2 inhibitor treatment or external irradiation. Furthermore, a separate subset of resistant blasts displayed robust p53 protein induction following MI-219 treatment, indicative of defective p53 protein function or defects in the apoptotic p53 network. Finally, analysis of very sensitive AML cases uncovered a strong and significant association with mutated Flt3 status (FLT3-ITD), which for the first time identified a clinically high risk group of AML that may particularly benefit from MDM2 inhibitor treatment.

In one embodiment of the methods provided herein, an MDM2 inhibitor and optionally one or more other anticancer agents are administered to a subject under one or more of the following conditions: at different periodicities, at different durations, at different concentrations, by different administration routes, etc. In another embodiment, the MDM2 inhibitor is administered prior to the other anticancer agent, e.g., 0.5, 1, 2, 3, 4, 5, 10, 12, or 18 hours, 1, 2, 3, 4, 5, or 6 days, or 1, 2, 3, or 4 weeks prior to the administration of the other anticancer agent. In another embodiment, the MDM2 inhibitor is administered after the other anticancer agent, e.g., 0.5, 1, 2, 3, 4, 5, 10, 12, or 18 hours, 1, 2, 3, 4, 5, or 6 days, or 1, 2, 3, or 4 weeks after the administration of the other anticancer agent.

In another embodiment, the MDM2 inhibitor and optionally another anticancer agent are administered concurrently but on different schedules, e.g., the MDM2 inhibitor is administered daily while the other anticancer agent is administered once a week, once every two weeks, once every three weeks, or once every four weeks. In another embodiment, the MDM2 inhibitor is administered once a week while the other anticancer agent is administered daily, once a week, once every two weeks, once every three weeks, or once every four weeks.

Compositions provided herein include all compositions wherein the compounds provided herein are present in an amount which is effective to achieve its intended purpose. While individual needs vary, determination of optimal ranges of effective amounts of each component is may be determined as described herein. Typically, the MDM2 inhibitor may be administered to a mammal, e.g. humans, orally at a dose of 0.0025 to 50 mg/kg, or an equivalent amount of the pharmaceutically acceptable salt thereof, per day of the body weight of the mammal being treated for disorders responsive to induction of apoptosis. In one embodiment, about 0.01 to about 25 mg/kg is orally administered to treat, ameliorate, or prevent such disorders. For intramuscular injection, the dose is generally about one-half of the oral dose. For example, a suitable intramuscular dose would be about 0.0025 to about 25 mg/kg, or from about 0.01 to about 5 mg/kg.

The unit oral dose may comprise from about 0.01 to about 1000 mg, for example, about 0.1 to about 100 mg of the MDM2 inhibitor. The unit dose may be administered one or more times daily as one or more tablets or capsules each containing from about 0.1 to about 10 mg, conveniently about 0.25 to 50 mg of the compound or its solvates.

In a topical formulation, the MDM2 inhibitor may be present at a concentration of about 0.01 to 100 mg per gram of carrier. In a one embodiment, the MDM2 inhibitor is present at a concentration of about 0.07-1.0 mg/ml, for example, about 0.1-0.5 mg/ml, and in one embodiment, about 0.4 mg/ml.

In addition to administering the MDM2 inhibitor as a raw chemical, an MDM2 inhibitor may be administered as part of a pharmaceutical preparation containing suitable pharmaceutically acceptable carriers comprising excipients and auxiliaries which facilitate processing of the compounds into preparations which can be used pharmaceutically. The preparations, particularly those preparations which can be administered orally or topically and which can be used for one type of administration, such as tablets, dragees, slow release lozenges and capsules, mouth rinses and mouth washes, gels, liquid suspensions, hair rinses, hair gels, shampoos and also preparations which can be administered rectally, such as suppositories, as well as suitable solutions for administration by intravenous infusion, injection, topically or orally, contain from about 0.01 to 99 percent, in one embodiment from about 0.25 to 75 percent of active compound(s), together with the excipient.

The compounds and pharmaceutical compositions disclosed herein may be administered to any patient who may experience the beneficial effects of the compounds. Foremost among such patients are mammals, e.g., humans, although the methods and compositions provided herein are not intended to be so limited. Other patients include veterinary animals (cows, sheep, pigs, horses, dogs, cats and the like).

The compounds and pharmaceutical compositions thereof may be administered by any means that achieve their intended purpose. For example, administration may be by parenteral, subcutaneous, intravenous, intramuscular, intraperitoneal, transdermal, buccal, intrathecal, intracranial, intranasal or topical routes. Alternatively, or concurrently, administration may be by the oral route. The dosage administered will be dependent upon the age, health, and weight of the recipient, kind of concurrent treatment, if any, frequency of treatment, and the nature of the effect desired.

The pharmaceutical preparations provided herein are manufactured in a manner which is itself known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example lactose or sucrose, mannitol or sorbitol, cellulose preparations and/or calcium phosphates, for example tricalcium phosphate or calcium hydrogen phosphate, as well as binders such as starch paste, using, for example, maize starch, wheat starch, rice starch, potato starch, gelatin, tragacanth, methyl cellulose, hydroxypropylmethylcellulose, sodium carboxymethylcellulose, and/or polyvinyl pyrrolidone. If desired, disintegrating agents may be added such as the above-mentioned starches and also carboxymethyl-starch, cross-linked polyvinyl pyrrolidone, agar, or alginic acid or a salt thereof, such as sodium alginate. Auxiliaries are, above all, flow-regulating agents and lubricants, for example, silica, talc, stearic acid or salts thereof, such as magnesium stearate or calcium stearate, and/or polyethylene glycol. Dragee cores are provided with suitable coatings which, if desired, are resistant to gastric juices. For this purpose, concentrated saccharide solutions may be used, which may optionally contain gum arabic, talc, polyvinyl pyrrolidone, polyethylene glycol and/or titanium dioxide, lacquer solutions and suitable organic solvents or solvent mixtures. In order to produce coatings resistant to gastric juices, solutions of suitable cellulose preparations such as acetylcellulose phthalate or hydroxypropylmethyl-cellulose phthalate, are used. Dye stuffs or pigments may be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer such as glycerol or sorbitol. The push-fit capsules can contain the active compounds in the form of granules which may be mixed with fillers such as lactose, binders such as starches, and/or lubricants such as talc or magnesium stearate and, optionally, stabilizers.

In soft capsules, the active compounds are in one embodiment dissolved or suspended in suitable liquids, such as fatty oils, or liquid paraffin. In addition, stabilizers may be added.

Possible pharmaceutical preparations which can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400. Aqueous injection suspensions may contain substances which increase the viscosity of the suspension include, for example, sodium carboxymethyl cellulose, sorbitol, and/or dextran. Optionally, the suspension may also contain stabilizers.

The topical compositions provided herein are formulated in one embodiment as oils, creams, lotions, ointments and the like by choice of appropriate carriers. Suitable carriers include vegetable or mineral oils, white petrolatum (white soft paraffin), branched chain fats or oils, animal fats and high molecular weight alcohol (greater than C₁₂). The carriers may be those in which the active ingredient is soluble. Emulsifiers, stabilizers, humectants and antioxidants may also be included as well as agents imparting color or fragrance, if desired. Additionally, transdermal penetration enhancers can be employed in these topical formulations. Examples of such enhancers can be found in U.S. Pat. Nos. 3,989,816 and 4,444,762.

Ointments may be formulated by mixing a solution of the active ingredient in a vegetable oil such as almond oil with warm soft paraffin and allowing the mixture to cool. A typical example of such an ointment is one which includes about 30% almond oil and about 70% white soft paraffin by weight. Lotions may be conveniently prepared by dissolving the active ingredient, in a suitable high molecular weight alcohol such as propylene glycol or polyethylene glycol.

The following examples are illustrative, but not limiting, of the methods of the methods and compositions provided herein. Other suitable modifications and adaptations of the variety of conditions and parameters normally encountered in clinical therapy and which are obvious to those skilled in the art are within the spirit and scope of the methods and compositions provided herein.

As part of broad-based efforts to study the therapeutic potential of MDM2 inhibitors in hematological malignancies (that are generally characterized by only a small fraction of cases with mutated p.53), ex vivo apoptosis induction in greater than 100 human AML samples has been studied. Through the studies detailed below, a previously unsuspected large fraction of primary human AML samples that displayed primary resistance to MDM2 inhibitors despite wild type p53 exon 5-9 gene status have been identified. Initial investigations into this phenomenon provide evidence for multiple distinct mechanisms of resistance: one centered on insufficient p53 protein induction and another centered on defective p53 protein or defective p53-regulated effector pathways. These novel findings substantially complicate transition of MDM2 inhibitors into clinical AML applications and motivate further study to achieve optimal efficaciousness of these drugs in the clinical setting. Finally through correlative analysis, a significant and strong association between mutated Flt3 status (presence of FLT3-ITD) and heightened sensitivity to MDM2 inhibitors has been identified for the first time, thus providing a novel and practical rationale for MDM2 inhibitor trial design, patient subgroup selection and trial data interpretation in AML.

### EXAMPLE 1

### Methods

### Patients

The 109 AML cases analyzed in this study were enrolled at the University of Michigan Comprehensive Cancer Center between March 2005 and October 2009. The study was approved by the University of Michigan Institutional Review Board (IRBMED #2004-1022), and written informed consent was obtained from all patients prior to enrollment.

### Cell Purification

Peripheral blood mononuclear cells from AML patients were isolated by Ficoll gradient centrifugation (GE Healthcare), aliquoted into FCS with 10% DMSO, and cryopreserved in liquid nitrogen. For purification of AML blasts using negative selection, cryopreserved PBMCs derived from AML patients were washed and recovered by centrifugation and then treated with anti-human CD3 (Miltenyi Biotec #130-050-101), anti-human CD14 microbeads (if blasts were negative for CD14 expression; Miltenyi Biotec #130-050-201), anti-human CD19 (if blasts were negative for CD19 expression; Miltenyi Biotec #130-050-301) and anti-human CD235a (Miltenyi Biotec #130-050-501) per manufacturer's recommendations. Cell suspensions were run through Miltenyi

MACS separation LS columns (#130-042-401) in order to negatively enrich for AML blasts. All blast preps were analyzed by cytospins for purity. This schema always resulted in greater than 90% blast purity.

AML blasts DNA used for SNP 6.0 profiling was extracted from samples that were further purified as follows: post-Miltenyi column samples were washed and stained with FITC-conjugated anti-CD33, PE-conjugated anti-CD13, and APC-conjugated anti-CD45 (all antibodies: eBioscience, San Diego, Calif.). After final washing, propidium iodide (PI) was added to a concentration of 1 μg/ml to discriminate dead cells. Sorting of cells was done on a FACS-ARIA high-speed flow cytometer (Becton Dickinson, Mountain View, Calif.). Live cells (PI-negative) were gated for blasts by identifying those cells with intermediate-intensity staining for CD45 and low- to moderate-intensity side scatter (Borowitz, M. J. et al., Am. J. Clin. Pathol.100: 534-540 (1993)). CD33 and CD13 were then used in order to further discriminate blasts versus erythroid lineage and mature myeloid lineage cells.

### Ex Vivo AML Blasts MDM2 Inhibitor Apoptosis Assays

Blasts enriched to >90% purity using methods detailed above were incubated in serum-supplemented RPMI medium at 2.5×10⁵ cells in 100 μl final volume in the presence of various concentrations of the MDM2 inhibitors MI-219 and MI-63 (range 0.625-20 μM) for 40 hours. Apoptosis and necrosis was measured for each treated blast aliquot using annexin-V/PI FACS-based readouts and values subsequently normalized to spontaneous death rates in untreated parallel cultures according to the formula (% alive=% mean alive treated samples/% mean alive paired non-treated samples).

### Ex Vivo AML Blasts Epigenetic and MDM2 Inhibitor Apoptosis Assays

Blasts enriched to >90% purity using methods detailed above were incubated with either DMSO or 0.5 μM 5-azacytidine (A2385, Sigma-Aldrich, Saint Louis, Mo.) for 48 hours (with 5-azacytidine replenished every 24 hours). During the last 12 hours of incubation, blasts were further aliquoted and treated with either 0.3 μM Trichostatin A (#9950, Cell Signaling Technology, Danvers, Mass.) or DMSO. At the end of the 48 hour incubation, each of the four differentially treated subgroups of blasts were treated with MI-219 at final concentrations of 0, 2.5, 5 and 10 μM for 40 hours, followed by annexin-V/PI FACS-based analysis of apoptosis. Aliquots of blasts in parallel were cultured in a 48-well plate at 10⁶ cells per well in 1 ml of medium and treated with 10 μM MI-219 or solvent for 8 hours. Blasts were harvested, lysed and protein prepared for immunoblotting as described.

Measurement of p53, MDM2 and MDMX mRNA Expression Using Q-PCR

RNA was prepared from FACS-sorted blasts from AML cases using the Trizol reagent and resuspended in 50 μl DEPC-treated water. Twenty μl complementary DNA was made from ˜50 ng of RNA using the Superscript III first strand synthesis kit (Invitrogen) and random priming. Primers and TaqMan-based probes were purchased from Applied Bio-systems (Primers-on-demand). Primer/probe mixtures included: p53 (Hs_{—}00153349_ml), MDM2 (Hs_{—}01066930_ml), MDM4 (Hs_{—}00159092_ml) and Hu PGK1.

Duplicate amplification reactions included primers/probes, TaqMan® 2× Universal PCR Master Mix, No AmpErase UNG and 1 μl of cDNA in a 20 ul reaction volume. Reactions were done on an ABI 7900HT machine. Normalization of relative copy number estimates for the mRNA of the gene of interest was done with the Ct values for PGK1 as reference (Ct mean gene of interest−Ct mean PGK1). Comparisons between AML subgroups were performed though subtractions of means of normalized Ct values.

### AML Blast Treatment Ex Vivo and Immunoblotting Procedures.

Primary AML cases with wild type p53 exon 5-9 were ranked according to the IC₅₀ values for MI-219 and 15 cases with high IC₅₀ values (IC₅₀>10 μM) and 15 cases with low IC₅₀ values (IC₅₀ values<2 μM) selected for further analysis. Blasts were purified as outlined above and subsequently cultured for 8 hours with either 10 μM MI-219, 10 μM Nutlin-3, solvent, or treated with 5 Gy of ionizing radiation. Cells were harvested post-treatment, lysed in detergent lysis buffer (50 mM Tris pH7.5, 100 mM NaCl, 2 mM EDTA, 2 mM EGTA, 1% Triton X-100, 20 mM NaF, 1 mM Sodium orthovanadate (#13721-39-6 Alfa Aesar), 1 mM Phenylmethanesulphonylfluoride (Pierce), phosphatase inhibitor cocktail I (P2850, Sigma-Aldrich) and protease inhibitor cocktail (P8340, Sigma-Aldrich)), protein fractionated, and prepared for immunoblotting with antibodies directed against p53 (Ab-6, clone DO-1, Calbiochem) and actin (AC-15, Sigma-Aldrich, Saint Louis, Mo.). Positive control lysates were generated from the AML cell line MOLM-13 treated with MI-219 at 10 μM for 8 hours, and aliquots of these lysates were run side-by-side with lysates of the primary cases on every immunoblot. Thus, these MOLM-13 lysates served as internal standards for blot-to-blot band intensity comparisons. Left-over lysates from these experiments were subsequently prepared for immunoblotting using antibodies against human MDMX (A300-287A, Bethyl Laboratories, Montgomery, Tex.), MDM2 (Ab-1, clone IF2, Calbiochem), p21 (clone SX118, BD Biosciences) and actin.

### SNP 6.0 Array Analysis of AML Blasts DNA and Paired Buccal DNA.

The SNP 6.0 assay was performed following manufacturer's recommended protocols. Affymetrix CEL files for each blast and buccal sample were analyzed using Affymetrix Genotyping Console software for initial quality control, followed by use of the Affymetrix “Birdseed” algorithm to generate tab-delimited SNP call files in text format. Call rates for the entire group of samples included in this report were between 94.93% and 99.45%, with a mean call rate of 98.33%. Sample copy number heatmap displays were obtained from CEL files through use of the freely available software dChip (Lin, M., Bioinformatics 20:1233-1240 (2004)) adapted to a 64-bit operating system environment. To generate functional and practical displays of LOH, a two-step, internally developed, Java-based software analysis system was employed. The Pre-LOH Unification Tool (PLUT) served to align all individual patient SNP calls to their respective dbSNP rs ID numbers and genomic physical positions prior to incorporation into the LOH tool version 2, an updated version of the LOH tool able to accommodate Affy 6.0 SNP array data (Ross, C. W. et al., Clin. Cancer Res.13: 4777-4785 (2007). For LOH analysis between paired samples, a filter setting within the LOH tool version 2 was employed, allowing visualization of individual paired SNP calls as LOH only if present within 3000 base pairs of another such call. This step filtered out many false, sporadically distributed single LOH calls due to platform noise.

Exon Resequencing of NPM1, Flt3, p53, N-ras, K-ras.

Primers to amplify and sequence exons 12 of human NPM1, exons 13-15 and 20 of human Flt3, exons 2 and 3 of N-ras and K-ras and exons 5-9 of human p.53 and adjacent intronic sequences were designed using the primer 3 program (http://frodo.wi.mit.edu/cgi-bin/primer3/primer3_www.cgi). PCR products were generated using Repli-g (Qiagen)-amplified DNA from highly pure blast cells as templates. Amplifications were done using Taq polymerase. PCR amplicons were prepared for direct sequencing with internal nested sequencing primers using the exonuclease/shrimp alkaline phosphatase method (USB). Mutation Surveyor (SoftGenetics LLC, State College, Pa.) software was used to compare experimental sequences against Refseq GenBank or genomic sequences as well as by visual inspection of sequence tracings. Mutations were confirmed using paired patient buccal DNA as templates.

### Statistical Methods.

Associations between binary classifications (e.g. between drug sensitivity and gene mutation status) were assessed using log odds ratios. Mean IC₅₀ values were compared between groups of samples using two-sample t-tests. Results for all statistical tests are reported as Z-scores and two-sided p-values.

### EXAMPLE 2

### Patient Characteristics

Characteristics of the 109 AML patients analyzed in this study are summarized in Table 1. Of the 109 AML cases analyzed, 90 (83%) were previously untreated and 19 (17%) were previously treated (relapsed) at study enrollment. Seventy percent, 14%, and 16% were either primary, secondary or treatment related AML (tAML), and 12 cases had p.53 exon 5-9 mutations.

TABLE 1
Baseline characteristics of patients

| | Treatment-naïve at | Previously treated at |
|---|---|---|
| Characteristic | enrollment, no. (%) | enrollment, no. (%) |
| Sample Size (N = 109) | 90 (83) | 19 (17) |
| Age, y | | |
| Median | 62 | 60 |
| Range | 20-85 | 24-79 |
| Sex | | |
| Male | 53 (59) | 11 (58) |
| Female | 37 (41) | 8 (42) |
| Pathogenesis | | |
| De novo | 61 (68) | 15 (80) |
| Prior myelodysplasis | 13 (14) | 2 (10) |
| Treatment-related | 16 (18) | 2 (10) |
| FAB classification* | | |
| M0 | 11 (12) | 0 (0) |
| M1 | 13 (14) | 4 (21) |
| M2 | 14 (16) | 2 (11) |
| M3 | 0 (0) | 0 (0) |
| M4 | 33 (37) | 6 (32) |
| M5 | 6 (7) | 3 (16) |
| M6 | 0 (0) | 0 (0) |
| M7 | 0 (0) | 0 (0) |
| Cytogenetic class** | | |
| Favorable | 6 (7) | 0 (0) |
| Intermediate | 48 (53) | 17 (90) |
| Unfavorable | 36 (40) | 2 (10) |
| No. of karyotypic | | |
| abnormalities | | |
| Three or more | 19 (21) | 0 (0) |
| Less than three | 71 (79) | 19 (100) |
| 5q-status | | |
| Present | 17 (19) | 0 (0) |
| Absent | 73 (81) | 19 (100) |
| 7q-status | | |
| Present | 6 (7) | 2 (10) |
| Absent | 84 (93) | 17 (90) |
| p53 exons 5-9 status | | |
| Mutated | 12 (13) | 0 (0) |
| Wildtype | 78 (87) | 19 (100) |
| Flt3 ITD status | | |
| ITD present | 12 (13) | 7 (37) |
| Wildtype or TK-835 | 78 (87) | 12 (63) |
| mutation (N = 2) | | |
| NPM1 status | | |
| Mutated | 12 (13) | 11 (58) |
| Wildtype | 78 (87) | 8 (42) |
| Induction type at diagnosis | | |
| Intensive therapy | None | 19 (100) |
| Anthracycline + cytarabine | | 18 (95) |
| only | | |
| Amonafide + cytarabine | | 1 (5) |

*The FAB classification was unspecified in seventeen samples.
**Based on the SWOG S0106 classification

### EXAMPLE 3

### Primary Resistance to MDM2 Inhibitor Treatment is Common in Adult AML

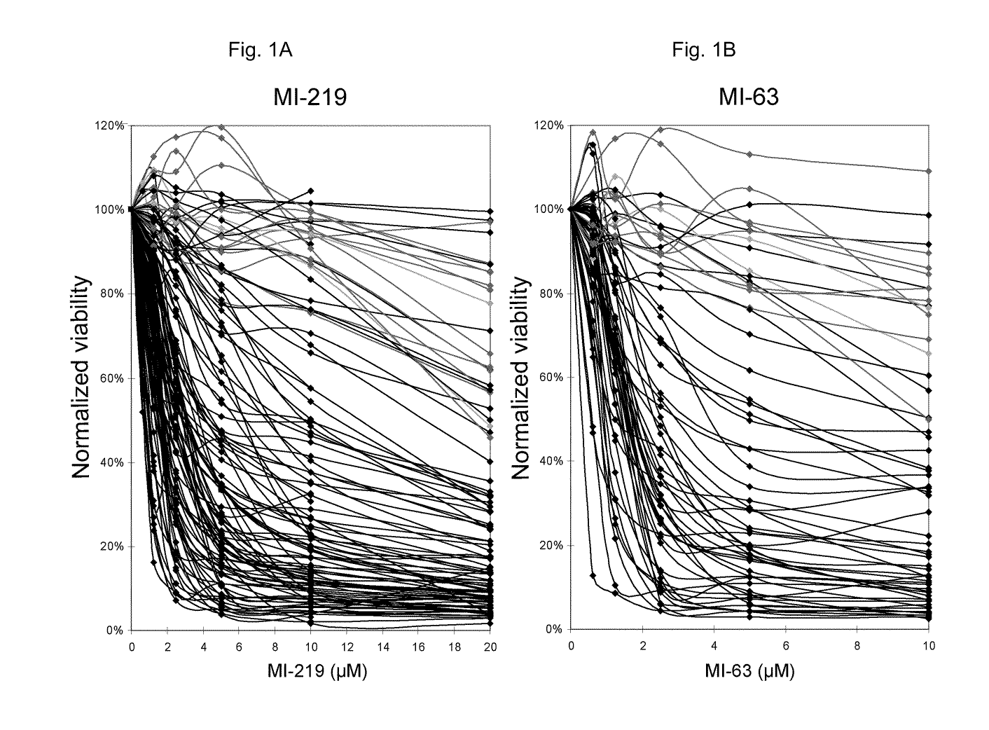

To evaluate the efficacy of MDM2 inhibitor-mediated apoptotic cell kill in AML blasts ex vivo, blasts from 109 AML specimens (97 with wild type p53 and 12 with mutant p53 by exon 5-9 exon sequence analysis) were purified to >90% purity and cell aliquots were incubated for 40 hours with escalating concentrations of the MDM2 inhibitors MI-219 (N=109) and MI-63 (N=60). The apoptotic cell fraction in treated samples was subsequently quantitated through annexin V-PI-based FACS analysis and normalized to measurements in paired untreated cells. As can be seen in FIGS. 1A and 1B, all AML cases with mutant p.53 exon 5-9 (red) or absent p53 mRNA expression (green) displayed resistance to MDM2-inhibitor treatment, consistent with previous findings (Kojima, K. et al, Blood 106: 3150-3159 (2005); Saddler, C. et al., Blood 111: 1584-1593 (2008).

While many AML cases with wild type p53 exon 5-9 (black) were very sensitive (IC₅₀<2 μM; 32/97=33%) or sensitive (IC₅₀>2 μM to <5 μM; 33/97=34%) to MI-219 or MI-63, a substantial fraction of cases with wild type p53 displayed varying resistance levels (MI-219 IC₅₀>5 μM for 32/97=33% and IC₅₀>10 μM for 21/97=22% of cases, respectively). Thus, unlike the situation in CLL, these data demonstrate that a substantial subset of AML cases with wild type p53 exon 5-9 sequence displays primary resistance to MDM2 inhibitors ex vivo.

Further, the mean IC₅₀ values for MI-219 in primary, secondary and tAML (exclusive of cases with p53 exon 5-9 mutations), were 6.1 μM, 7.9 μM and 4.8 μM, respectively. The mean IC₅₀ values for MI-219 in previously untreated versus relapsed AML cases (exclusive of cases with p53 exon 5-9 mutations) was 6.6 μM versus 4.4 μM, respectively.

### EXAMPLE 4

### Varying Degrees of Sensitivity and Resistance to MDM2 Inhibitors in AML Cell Lines

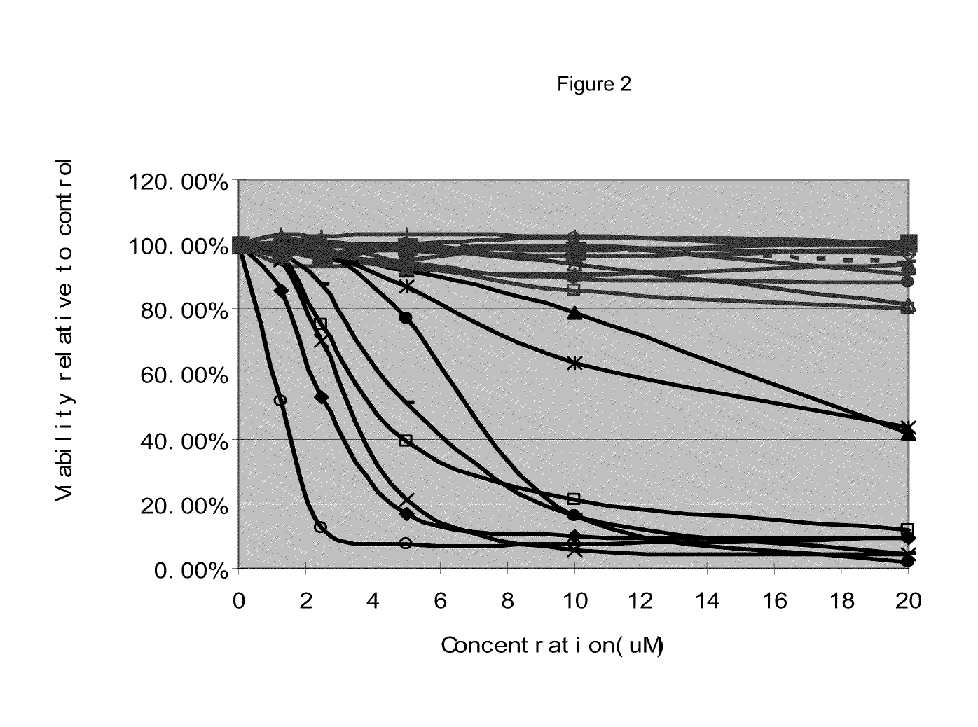

Next—the ability of MI-219 to induce apoptosis in 19 AML-derived cell lines was assessed. Data are summarized in FIG. 2 and Table 2.

As can be seen in FIG. 2, all AML cell lines with mutant p53 (red) were resistant to MI-219, while the cell lines with wild type p53 (black) displayed varying degrees of sensitivity/resistance to MI-219, reminiscent of the findings in primary AML blasts presented above.

TABLE 2

| | | p53 | immunoblot | | | |
|---|---|---|---|---|---|---|
| AML cell | FAB | (exon5-9) | (MI-219 | IC₅₀/MI-219 | IC₅₀/MI-63 | IC₅₀/Nutlin |
| line | Type | status | 10 μM) | (μM) | (μM) | (μM) |
| SKM-1 | sM5 | mut | non-inducible | >20 | >10 | >10 |
| OCI-M2 | sM6 | mut | non-inducible | >20 | >10 | >10 |
| MONO-MAC-6 | sM5 | mut | non-inducible | >20 | >10 | >10 |
| GF-D8 | M1 | mut | non-inducible | >20 | >10 | >10 |
| MOLM-16 | M0 | mut | non-inducible | >20 | >10 | >10 |
| Kasumi-3 | M0 | mut | non-inducible | >20 | >10 | >10 |
| Kasumi-1 | M2 | mut | non-inducible | >20 | >10 | >10 |
| ME-1 | M4eo | mut | null | >20 | >10 | >10 |
| AML-193 | M5 | mut | null | >20 | >10 | >10 |
| KG-1 | M6 | mut | null | >20 | >10 | >10 |
| HL60 | M2 | deleted | null | >20 | >10 | >10 |
| OCI-AML-3 | M4 | wt | Inducible | 18.176 | >10 | >10 |
| OCI-AML-5 | M4 | wt | Inducible | 13.846 | >10 | >10 |
| ML-2 | sM4 | wt | Inducible | 7.038 | 7.468 | 10.447 |
| OCI-AML-2 | M4 | wt | Inducible | 5.248 | 4.735 | 7.216 |
| AP-1060 | M3 | wt | Inducible | 4.224 | 4.029 | 6.768 |
| SIG-M5 | M5a | wt | Inducible | 3.436 | 3.801 | 4.727 |
| MOLM-13 | sM5a | wt | Inducible | 2.657 | 2.855 | 4.044 |
| MUTZ-2 | M2 | wt | Inducible | 1.283 | 1.17 | 0.951 |

### EXAMPLE 5

### Evidence for Distinct Mechanisms of Primary Resistance to MDM2 Inhibitors in AML with Wild Type p53 Exon 5-9

Given the central importance of intact p53 to MDM2 inhibitor sensitivity, analysis of p53 protein expression levels in primary AML blasts was conducted. All primary AML cases with wild type p53 exon 5-9 were ranked according to the IC₅₀ values for MI-219 and 15 cases with high IC₅₀ values (IC₅₀>10 μM) and 15 cases with low IC₅₀ values (IC₅₀ values<2 μM) were selected for further analysis. Purified blasts were either left untreated or treated for 8 hours with MI-219 (10 μM), Nutlin 3 (10 μM) or a one time dose of 5 Gy of external irradiation. Cellular lysates made from these blasts were prepared for immunoblotting with anti-p53 and anti-actin antibodies. Further, aliquots of lysates from the MI-219-treated AML cell line MOLM13 were analyzed on each blot to permit blot-to-blot comparisons of band intensities.

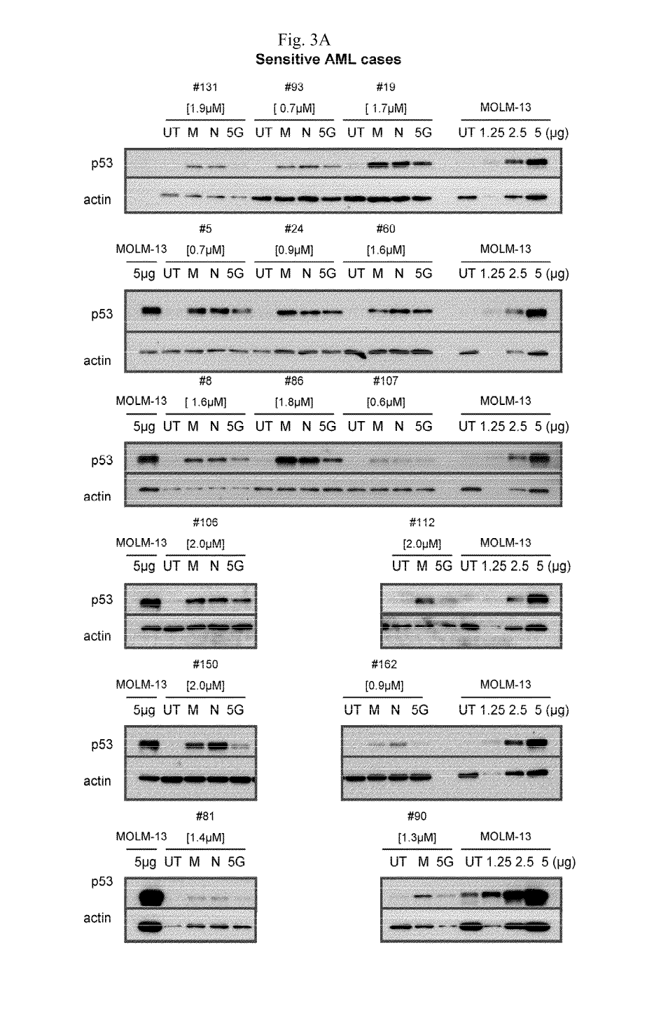

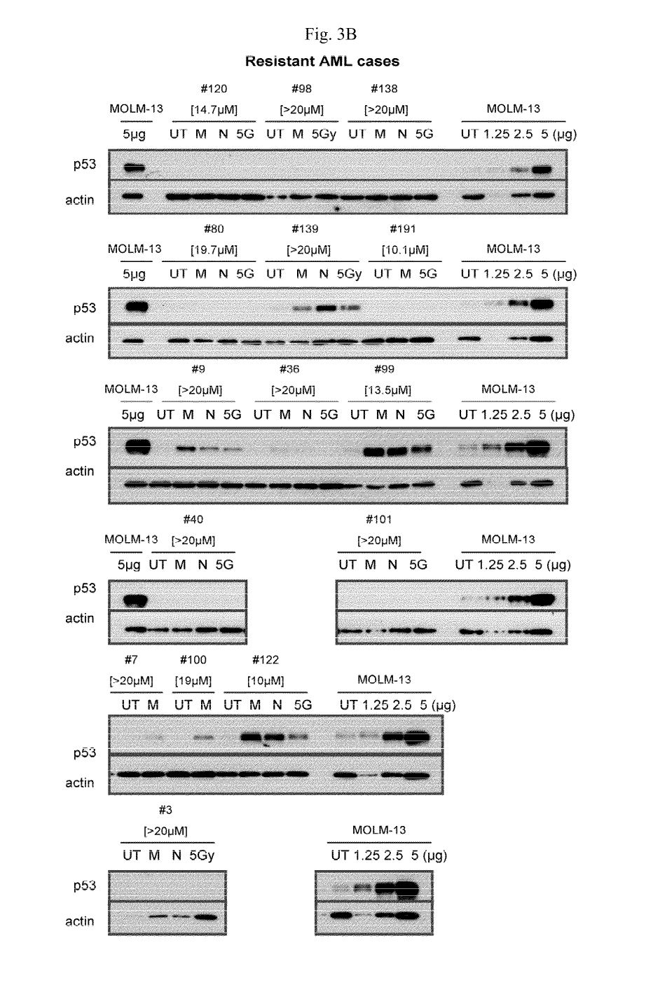

As can be seen in FIG. 3A, all sensitive AML blasts demonstrated induction of p53 protein after MDM2 inhibitor treatment or external irradiation, albeit to different absolute levels. Importantly, analysis of p53 protein levels in resistant blasts (FIG. 3B) revealed two subsets: i) blasts with absent or very low p53 expression after MDM2 inhibitor treatment or external irradiation and ii) blasts with baseline and induced p53 levels essentially equal to the levels measured in sensitive blasts. Thus, resistance to MDM2 inhibitors in AML with wild type p53 exon 5-9 is associated with at least two distinct molecular defects: i) low/absent p53 protein expression or, ii) apoptotic p53 network defects (including the possibility of aberrant p53 proteins) in the setting of normal p53 protein levels.

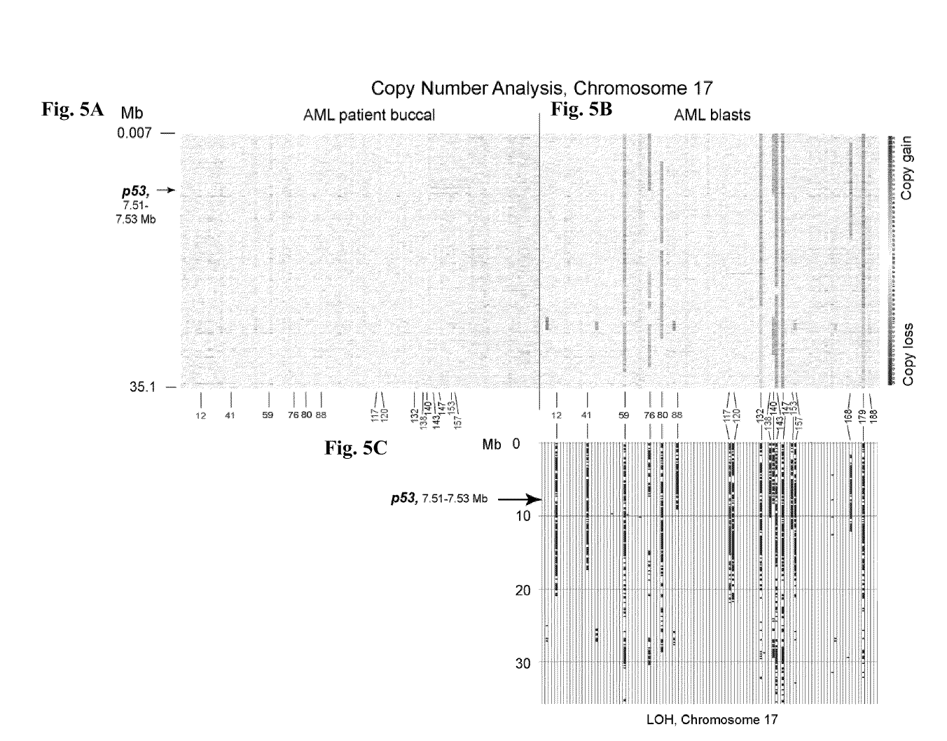

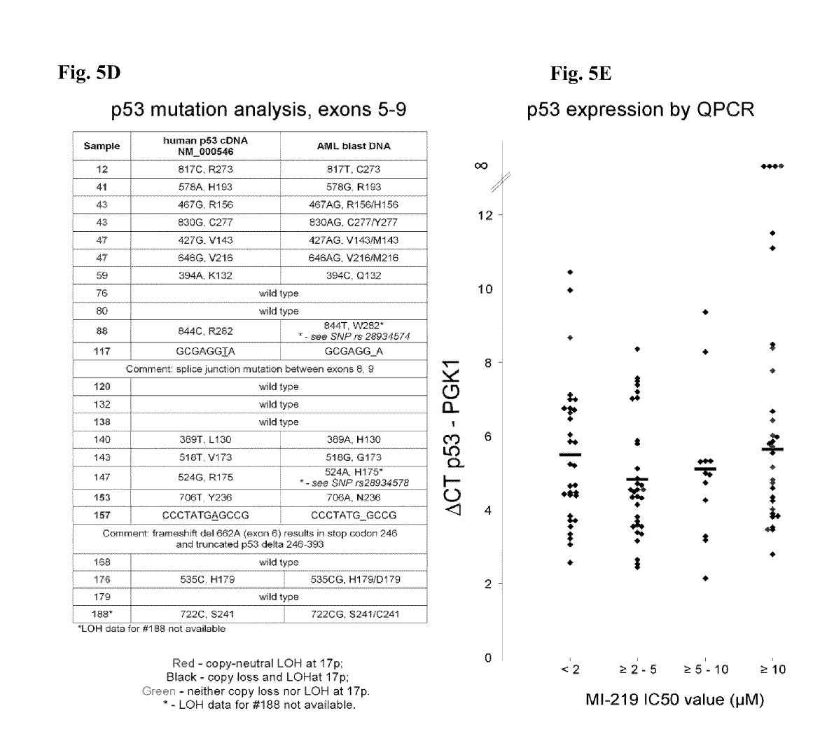

To gain further insights into the mechanisms of low/absent p53 expression in resistant AML blasts, normalized p53 mRNA levels in total RNA purified from FACS-sorted AML blast samples was measured. This analysis disclosed that a few AML cases at baseline displayed absent p53 mRNA (FIG. 5E; AML cases #7, 80 and 120). Thus, resistance to MDM2 inhibitors in a small fraction of AML blasts is due to absent p.53 transcription and suggests an acquired p53 gene defect. However, the majority of resistant AML blasts with low/absent p53 protein did express p53 mRNA (AML cases #98, 138, 191, 36, 40, 101 and 100) thus implying post-transcriptional mechanisms for low p53 protein levels.

### EXAMPLE 6

### The Treatment of Resistant Blasts with Absent p53 Expression Using Trichostatin A and Azacytidine Does Not Induce p53 Expression.

Attempting to obtain evidence for epigenetic p.53 gene silencing in AML with absent p53 mRNA expression, four AML cases were selected based on the availability of cryopreserved cells with absent or very low p53 mRNA for further analysis and treated purified blasts with trichostatin A and azacytidine (alone or in combination) followed by treatment with MI-219. Readouts for these experiments was the fraction of blasts undergoing apoptosis and posttreatment p53 protein levels. As detailed in FIG. 1, evidence for reversible epigenetic p.53 gene silencing in these blasts was not found.

### EXAMPLE 7

### Varying Expression Levels of MDM2 and MDMX Do Not Account for the Resistance to MDM2 Inhibitors in AML

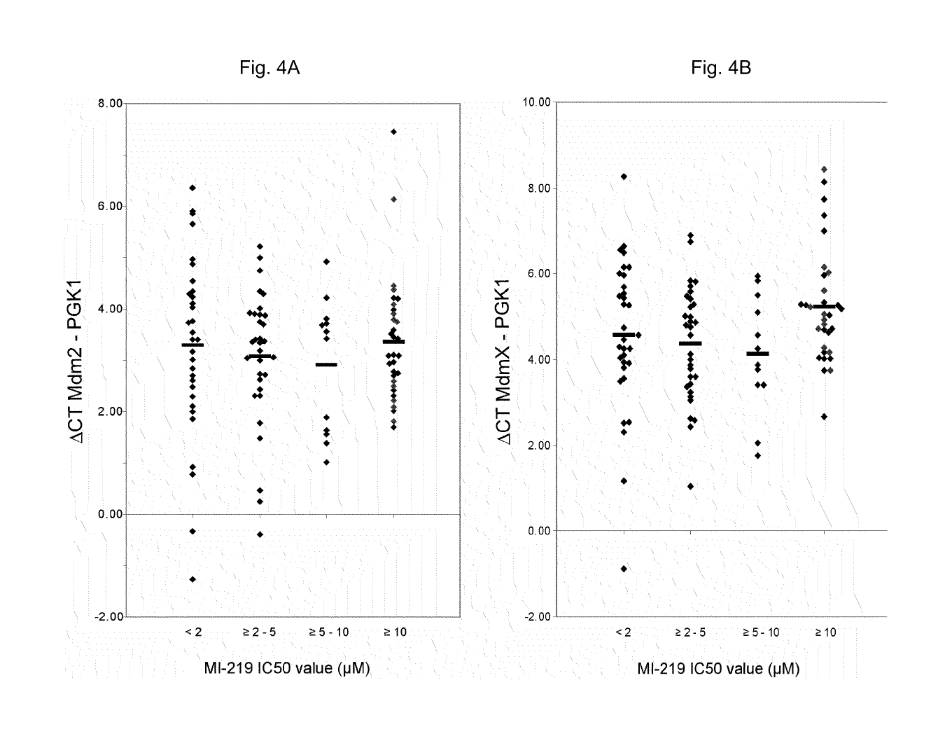

The expression levels of MDM2 and MDMX, two critical regulators of p53 protein, could account for the observed differences in IC₅₀ values to MDM2 inhibitor treatment in AML cases with wild type p53 exon 5-9 and for the observed differences in p53 protein levels. To test such hypotheses, normalized MDM2 and MDMX mRNA levels across the entire AML cohort was measured initially. Subsequently, these mRNA levels were correlated with IC₅₀ values to MDM2 inhibitor-mediated apoptosis in all AML cases with wild type p53 exon 5-9 (FIGS. 4A and 4B).

As can be seen in FIGS. 4A and 4B, neither MDMX nor MDM2 levels correlated with MI-219 IC₅₀ values. For instance, the mean delta Ct mean MDMX-PGK1 value was 5.2 for the AML cases with wild type p53 and MI-219 IC₅₀ values>10 μM and 4.4 for the AML cases with wild type p53 and MI-219 IC₅₀ values<10 μM, indicating slightly lower (˜1.7-fold) MDMX levels in resistant as compared with less resistant or sensitive blasts.

Focusing on MDM2, the mean delta Ct mean MDM2-PGK1 value was 3.1 for the AML cases with wild type p53 and MI-219 IC₅₀ values >10 μM and 3.2 for the AML cases with wild type p53 and MI-219 IC₅₀ values <10 μM, indicative of equal MDM2 mRNA levels in resistant as compared with less resistant and sensitive blasts.

Next MDMX, MDM2 and p21 protein levels were measured in lysates from blasts derived from the experiment outlined in FIG. 3. As can be seen in FIGS. 2 and 3, neither MDMX, MDM2 or p21 protein levels demonstrated a clear association with MI-219 IC₅₀ values.

### EXAMPLE 8

### Acquired Uniparental Disomy (Copy-Neutral LOH) is Common in AML and is Often Associated with p53 Mutations or Absent p53 Expression

To obtain additional information regarding p53 gene status in AML, DNA samples from ultra-pure AML blast populations from 110 AML cases were analyzed and paired buccal DNA for acquired chromosomal copy number alterations and LOH using ultra high density Affymetrix 6.0 SNP arrays.

In FIG. 5, shows sub-chromosomal copy number status at 17p (panel A buccal DNA, panel B AML blast-derived DNA; p53 is located at ˜7.5 Mb physical position on 17p), LOH at 17p (panel C), p53 exon 5-9 sequence data (panel D) and normalized p53 mRNA data (panel E). As can be seen, 17/110=15% of AML cases displayed LOH involving parts or all of 17p that spans the p53 locus. Importantly, paired analysis for copy loss uncovered 2N status for nearly half (8) of these cases (red numbering): examples of copy-neutral LOH or acquired uniparental disomy (aUPD) at 17p. Of note, copy-neutral LOH is undetectable using conventional karyotyping or CGH and is thus missed in routine clinical practice. Given that copy-neutral LOH is often associated with gene mutations, LOH data was compared with p53 sequence data and p53 mRNA data and found that 6/8 of these 17p-associated aUPD cases (red) displayed homozygous p.53 mutations (AML #12, 41, 88, 117, 153 and 157, panel D) and 1/8 cases (#120) had very little p53 mRNA expression. Thus, acquired copy-neutral LOH is common at the p.53 locus, is associated with p53 null states in the majority of cases and is associated with resistance to MDM2 inhibitor treatment. High resolution copy number analysis of the p.53 gene and the p.53 promoter did not identify homozygous deletions in AML.

### EXAMPLE 9

### FLT3-ITD is Associated with Enhanced Sensitivity to MDM2 Inhibitor Treatment in AML

Analysis of ex vivo sensitivities to MDM2 inhibitors outlined above revealed many cases that were very sensitive to MDM2 inhibitor-mediated apoptosis. Identification of markers that would correlate with increased MDM2 inhibitor sensitivity was pursued. Focusing on Flt3 and NPM1 (the two most commonly mutated genes in AML) the presence or absence of Flt3-ITD or NPM1 exon 12 mutations was correlated with MI-219 IC₅₀ values initially in all AML with wild type p53 exon 5-9. The AML cohort was repeatedly dichotomized at either the 25^{th.} or 50^{th.} percentile (corresponding to threshold IC₅₀ values of 1.78 μM and 3.2 μM, respectively) and determined Z-scores for presence of mutated Flt3 (FLT3-ITD) or NPM1, respectively. From this analysis Flt3-ITD emerged as significantly enriched in sensitive AML cases, with Z-scores of 1.91 (p=0.06) and Z=2.26 (p=0.02) for the 25^{th.} and 50^{th.} percentile analysis, respectively. Eleven out of 19 (58%) and 13 out of 19 (68%) Flt3-ITD-mutated AML cases had IC₅₀ values to MDM2 inhibitors of <2 μM and <2.25 μM, respectively.

A similar analysis for the comparison of FLT3-ITD positive cases versus all other cases (N=90; including p53 exon 5-9 mutated cases) was performed. From this analysis Flt3-ITD again emerged as significantly enriched in sensitive AML cases, with Z-scores of 2.42 (p=0.02) and Z=(p=0.01) for the 25^{th.} and 50^{th.} percentile analysis, respectively.

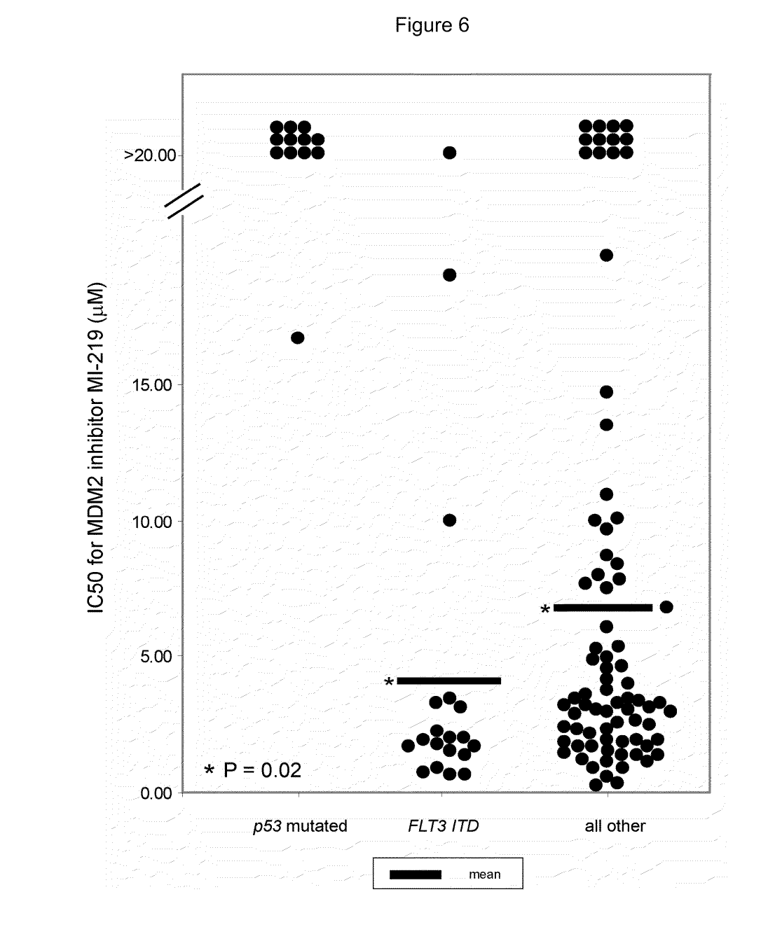

IC₅₀ values for all 109 AML cases are graphically displayed in three mutually exclusive categories: 1) presence of p53 exon 5-9 mutations, 2) presence of Flt3-ITD and 3) all others (see FIG. 6). As can be seen in FIG. 6, most Flt3-ITD positive AML cases displayed very low IC₅₀ values and significantly lower mean IC₅₀ values than the Flt3 wt and p53 wt group (P=0.02). Thus, the majority of AML blasts with Flt3-ITD mutations are highly sensitive to MDM2 inhibitor treatment. Therefore, this analysis identifies for the first time a clinically relevant genomic biomarker for MDM2 inhibitor sensitivity.

This report summarizes detailed studies of the molecular determinants in primary AML blasts (N=109) and their influence on sensitivity or resistance to MDM2 inhibitor treatment ex vivo. One finding of this study is the description and quantitative analysis of primary resistance to MDM2 inhibitors in AML. Within this context, it was demonstrated that: i) p.53 mutations confer resistance to MI-219, as expected, ii) low or absent p53 expression in the absence of p53 exon 5-9 mutations exists in a subset of AML blasts and is associated with MDM2 inhibitor resistance, and iii) MDM2 inhibitor resistance exists in subsets of AML despite wild type p53 and robust p53 protein induction following MDM2 inhibitor treatment or irradiation, thus implying defects in the apoptotic p53 network or in the p53 protein. Together these various AML blast-intrinsic defects result in primary resistance to MDM2 inhibitors in approximately one third of all AML cases; a fraction much larger than previously appreciated.

Regarding the p53 gene status of resistant AML blasts, multiple findings emerged: i) the p53 exon 5-9 mutation frequency was 10%, which is in line with previous estimates (Fenaux, P. et al., Blood 78: 1652-1657 (1991); Stirewalt, D. L. et al., Blood 97: 3589-3595 (2001)), and is insufficient to explain MDM2 inhibitor resistance in the majority of AML cases, ii) p53 mutations frequently (-50% of all p53 mutations) occurred in the setting of acquired UPD at 17p in AML, iii) some AML cases with 17p deletions that spanned p53 carried wild type p53 and were sensitive to MI-219, and, iv) some AML cases with 17p deletions that spanned p53 lacked p53 mRNA expression and thus were resistant to MI-219. Therefore, substantial combinatorial molecular diversity exists in the p53 gene status in AML with direct effects on the ability of MDM2 inhibitors to effect apoptotic AML blast death (Kojima, K. et al., Blood 106: 3150-3159 (2005); Seifert, H. et al., Leukemia 23: 656-663 (2009)).

Focusing on the resistant AML cases with wild type p53 exon 5-9 and presence of p53 mRNA, two subsets emerged that displayed either: i) low or absent p53 proteins or ii) preserved p53 protein levels. Regarding the molecular basis for low p53 protein in subsets of AML blasts, initial analysis did not identify supportive evidence for reversible epigenetic changes. It is possible that a defective p53 gene (including alterations in the promoter or epigenetic changes that defied pharmacological attempts at reversal), impaired p53 mRNA translation or reduced p53 protein stability that is independent of MDM2 or MDMX levels (Naski, N. et al., Cell Cycle 8: 31-34 (2009); Ofir-Rosenfeld, Y. et al., Mol. Cell. 32: 180-189 (2008); Maclnnes, A. W. et al., Proc. Natl. Acad. Sci. U.S.A. 105: 10408-10413 (2008); Takagi, M. et al., Cell 123: 49-63 (2005); Asher, G. et al., Proc. Natl. Acad. Sci. U.S.A. 99: 3099-3104 (2002); Leng. R. P. et al., Cell 112: 779-791 (2003); Dornan, D. et al., Nature 429: 86-92 (2004)).

Given the scarcity of the primary source material, this was not investigated further but raises questions regarding correlations between p.53 status and p53 protein levels that should be evaluated in future studies. Regarding the AML blasts with robust induction of p53 protein after MDM2 inhibitor treatment or external irradiation, the two principal molecular defects are i) a defective p53 protein, possibly due to aberrant post-translational modifications of p53, resulting in an altered ability of p53 to activated apoptotic signaling pathways (Knights, C. D. et al., J. Cell. Biol. 173: 533-544 (2006); Di Giovanni, S. et al., EMBO J. 25: 4084-4096 (2006); Murray-Zmijewski, F. et al., Nat. Rev. Mol. Cell. Biol. 9: 702-712 (2008)) or ii) defects in the p53-regulated apoptotic network. Regarding the latter possibility of defects in the p53 apoptotic network, it is important to note that a quantitative analysis of the relative importance of various p53 inducible genes in relation to the apoptotic response following p53 induction is not available. In the setting of RITA treatment of cells (but not Nutlin treatment) it has recently been demonstrated that p21 downregulation provides a switch between p53-induced apoptosis and cell cycle arrest (Enge, M. et al., Cancer Cell 15: 171-183 (2009)).

Analysis of p21 protein levels before and after Nutlin or MI-219 treatment also does not provide clear evidence for a unique role of p21 in AML blast fate decision following MDM2 inhibitor treatment. The initial analysis of gene expression changes in sensitive versus resistant blasts following treatment with MI-219 (not shown) identified differential induction of a subset of classical p53-responsive genes but interpretation of this data is hampered by the fact that the critical genes with importance to p53-mediated apoptosis in leukemia are not known. It is thus possible that genes other than classical p53-responsive genes are involved in conferring resistance to MDM2 inhibitors, and future analysis of such genes are of importance for a comprehensive understanding of MDM2 inhibitor effects on myeloid leukemia blasts.

One of the results from this analysis was the identification of AML blasts that were very sensitive to MI-219 ex vivo. Approximately one third of AML cases displayed IC₅₀ values to MI-219 of <2 μM. Attempts at identification of determinants of such heightened sensitivity uncovered frequent and significant association with mutated FLT3 (presence of FLT3-ITDs). For instance, 58% and 84% of all AML samples with FLT3-ITD (N=19) displayed MI-219 IC₅₀ values of <2 μM and <5 μM, respectively. Therefore, activated Flt3 appears to sensitize AML blasts to MDM2 inhibitor-mediated apoptosis, a finding that could be exploited for clinical applications of MDM2 inhibitors in AML.

In summary, this unique dataset, based on the analysis of >100 primary AML cases, quantitatively describes primary resistance to MDM2 inhibitors in AML and provides evidence for multiple distinct molecular mechanisms. These unexpected findings thus substantially complicate transition of MDM2 inhibitors into AML therapy and provide the rationale for further in-depth pre-clinical studies of resistance mechanisms in AML. These studies also provide a rationale for combination targeted therapy in AML with primary resistance to MDM2 inhibitors in an attempt to overcome resistance (Kojima, K. et al., Leukemia 22: 1728-1736 (2008)).

Conversely, the finding of an association of mutated FLT3 (FLT3-ITD) and heightened sensitivity to MDM2 inhibitors was not expected based on published findings (Kojima, K. et al., Leukemia 24: 33-43 (2010)) and may be important to AML therapy as: i) FLT3-ITD AML cases tend to have short remission durations, ii) therapeutic blockage of FLT3 using FLT3 inhibitor monotherapy has not yet resulted in substantial clinical benefits to patients and iii) application of MDM2 inhibitors to AML with mutated Flt3 and intact p53 will offer clinical benefits (Bacher, U. et al., Blood 111: 2527-2537 (2008)). This description of a genomic biomarker for MDM2 inhibitor sensitivity thus introduces the concept of “MDM2 inhibitor sensitizer gene mutations” and justifies ongoing searches for additional genes with similar effects. Finally, such MDM2 inhibitor sensitizer mutations will also offer explanations for the heightened sensitivity of neoplastic cells to MDM2 inhibitor treatment.

### EXAMPLE 10

### Cell Growth Inhibition and Cytotoxic Effects on MV4-11 and MOLM-13 AML Cell Lines

The compounds of Chart 3 were evaluated for their cell growth inhibition and cytotoxic effects on 2 AML cell lines: MV4-11 and MOLM-13 (from The DSMZ—Deutsche Sammlung von Mikroorganismen and Zellkulturen GmbH, reference DSMZ ACC554 and DSMZ ACC102, respectively). Both of these cell lines have the FLT3-ITD mutation. (Quentmeier, H. et al. Leukemia 17:120-124 (2003)). For growth inhibition assays, cells were incubated with the compounds of Chart 2 for 96 h in 96-well format. Cell seeding conditions were adapted to get significant cell growth in this assay format. Growth inhibition assays were performed using the Celltiter-Glo Luminescent kit (Promega). The IC₅₀ values (concentration where the growth inhibition percentage is equal to half of the maximum inhibitory effect of the tested compound) were calculated and ranged between 10 nM and 100 nM in the 2 AML cell lines for both compounds.

For cytotoxicity assays, cells were incubated with the compounds of Chart 2 for 96 h in 6-well format. Cell seeding conditions were adapted to get significant cell growth in this assay format. Cytotoxicity effects were performed using trypan blue staining Both the floating and adherent cells were stained with trypan blue. Quantification was performed by Vi-CELL® Cell Viability Analyzer (Beckmann-Coulter) which determines both cell concentration and percentage of viable cells. For both compounds of Chart 2 at concentrations which were close to IC₉₀ concentrations (concentration where the growth inhibition percentage is equal to 90 percent of the maximum inhibitory effect of the tested compound), the percentages of viable cells were significantly decreased compared to untreated cells and were at best between 50 and 70% in the MV4-11 cell line and below 50% for the MOLM-13 cell line.

The breadth and scope of the present methods and compositions provided herein should not be limited by any of the above-described exemplary embodiments, but should be defined only in accordance with the following claims and their equivalents.

The foregoing description of the specific embodiments will so fully reveal the general nature of the methods and compositions provided herein that others can, by applying knowledge within the skill of the art, readily modify and/or adapt for various applications such specific embodiments, without undue experimentation, without departing from the general concept of the methods and compositions provided herein. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

All patents, patent applications, and publications cited herein are fully incorporated by reference herein in their entirety.

1. A method of treating a patient having leukemia, the method comprising administering a therapeutically effective amount of a MDM2 inhibitor to the patient, wherein cells of the patient contain a FLT3 having an activating mutation.

2. A method of selecting a patient having leukemia for treatment with a MDM2 inhibitor, the method comprising:
(a) obtaining a biological sample from the patient;
(b) determining whether the biological sample contains a FLT3 having an activating mutation; and
(c) selecting the patient for treatment if the biological sample contains a FLT3 having an activating mutation.

3. The method of claim 2, further comprising administering a therapeutically effective amount of the MDM2 inhibitor to the patient.

4. A method of predicting treatment outcome in a patient having leukemia, the method comprising:
(a) obtaining a biological sample from the patient; and
(b) determining whether the biological sample contains a FLT3 having an activating mutation;
wherein the detection of a FLT3 having an activating mutation indicates that administering a therapeutically effective amount of a MDM2 inhibitor to the patient will cause a favorable therapeutic response.

5. A method of treating a patient having leukemia, the method comprising:
(a) obtaining a biological sample from the patient;
(b) determining whether to biological sample contains a FLT3 having an activating mutation; and
(c) administering a therapeutically effective amount of a MDM2 inhibitor to the patient if the biological sample contains a FLT3 having an activating mutation.

6. The method of any one of claims 2-5, wherein the biological sample comprises blood cells.

7. The method of any one of claims 2-5, further comprising determining whether the biological sample contains one or more p53 mutations.

8. The method of any one of claims 1-5, wherein the FLT3 activating mutation is an internal tandem duplication.

9. The method of any one of claims 1-5, wherein the patient is human.

10. The method of any one of claims 1-5, wherein the leukemia is acute myeloid leukemia.

11. The method of any one of claims 1-5, wherein the MDM2 inhibitor is a spiro-oxindole MDM2 inhibitor.

12. The method of claim 11, wherein the spiro-oxindole MDM2 inhibitor is selected from the group consisting of: 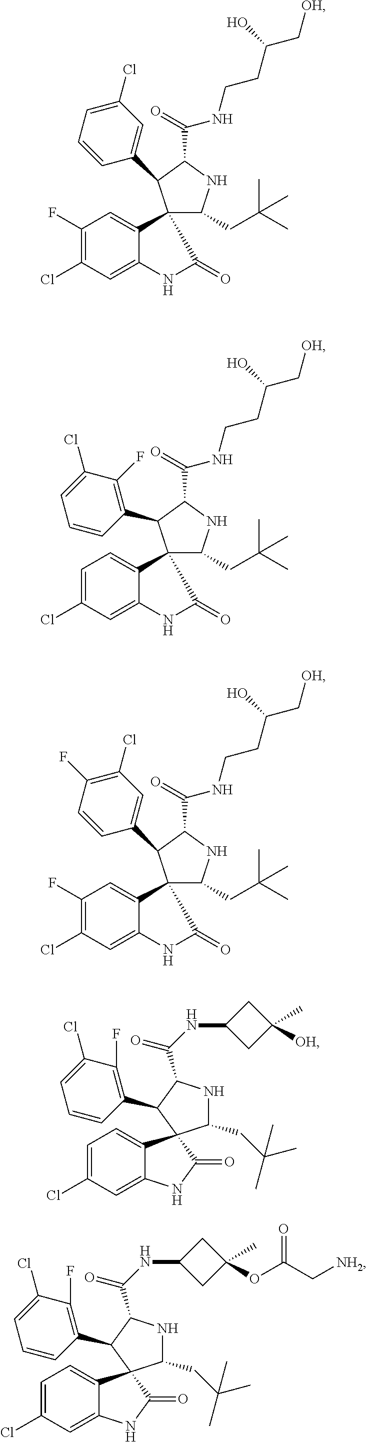 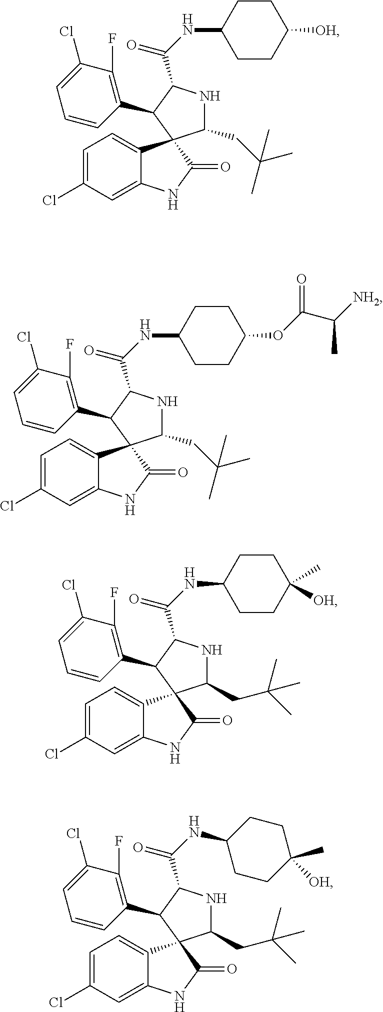 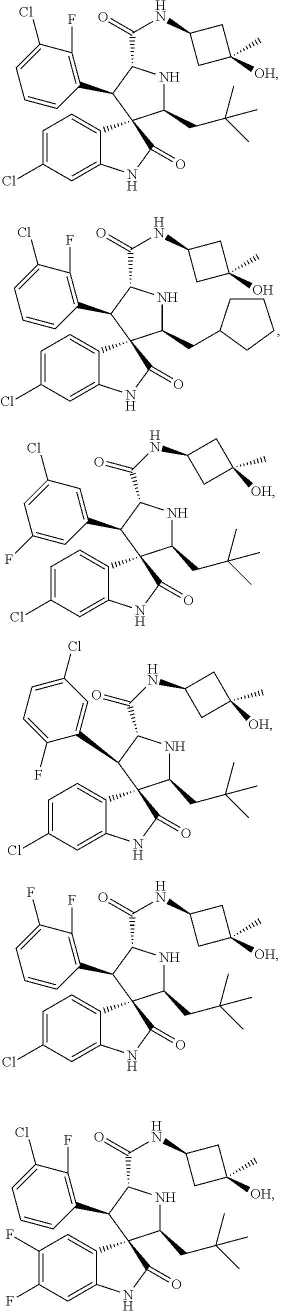 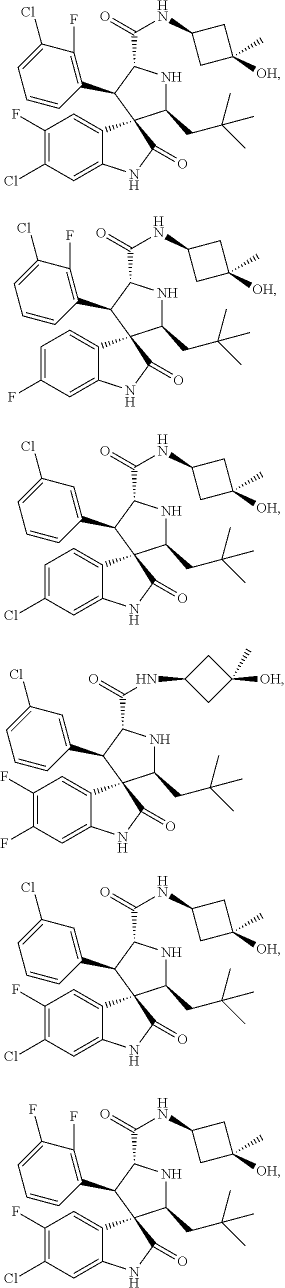 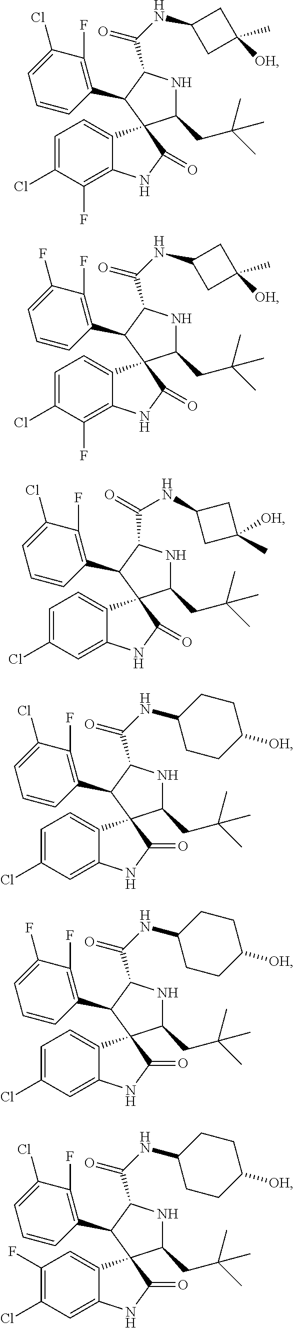 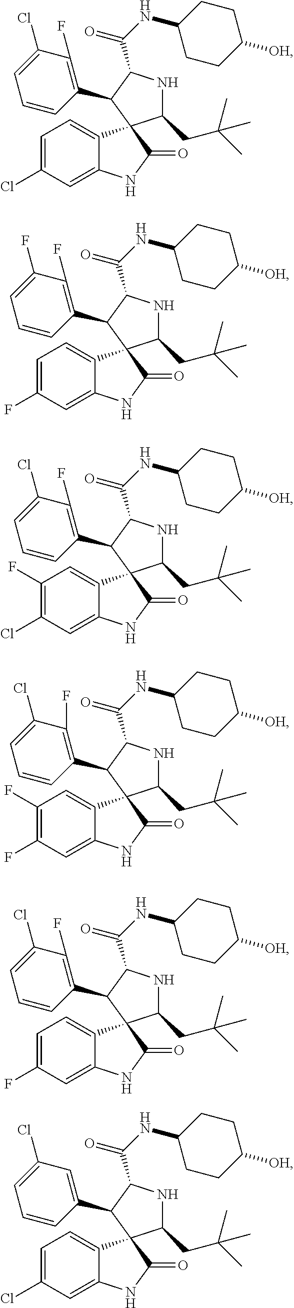 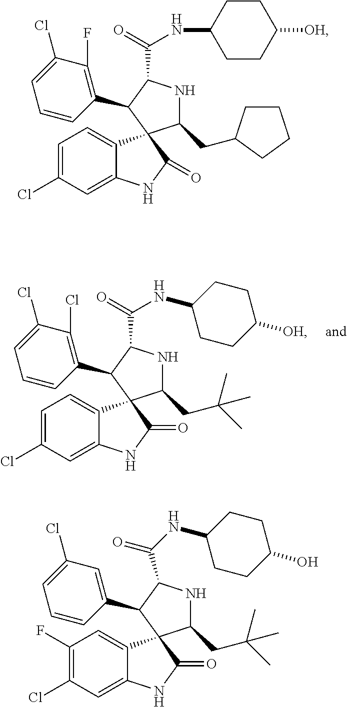
or a pharmaceutically acceptable salt thereof.

13. The method of any one of claim 1 or 3-5, wherein at least one additional anticancer agent is administered to the patient.

14. The method of claim 13, wherein the at least one additional anticancer is a FLT3 inhibitor.

15. A method of treating a human patient having acute myeloid leukemia, the method comprising administering a therapeutically effective amount of a compound selected from the group consisting of: 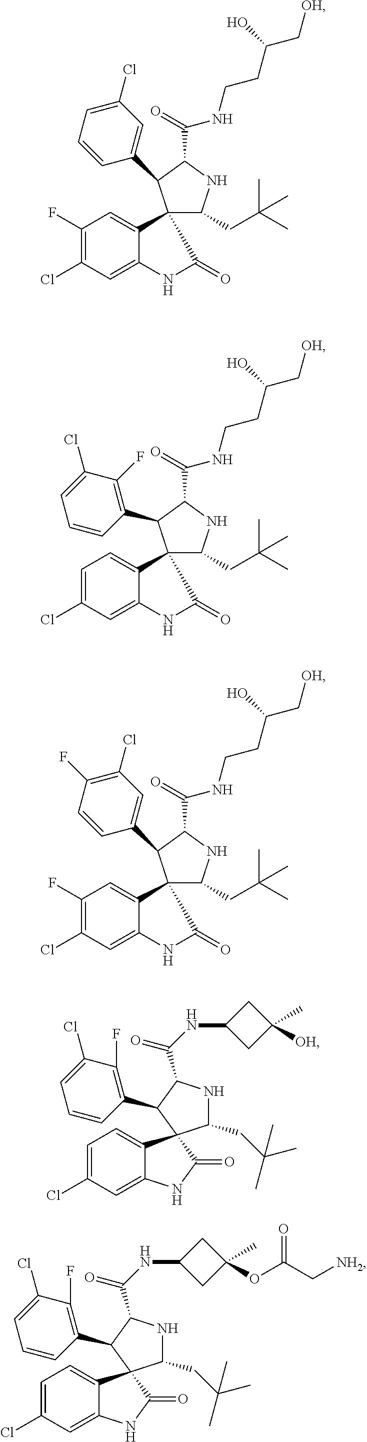 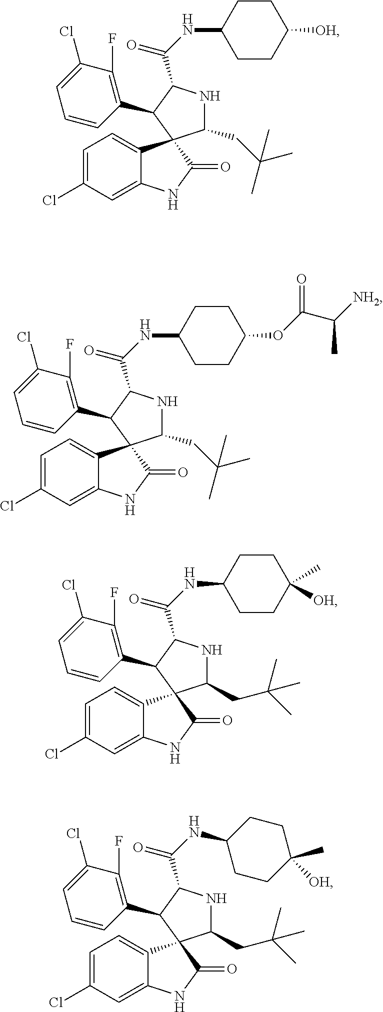 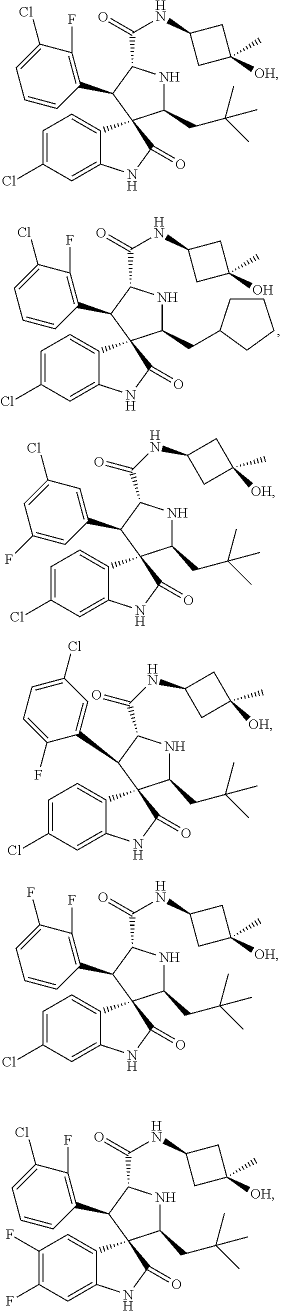 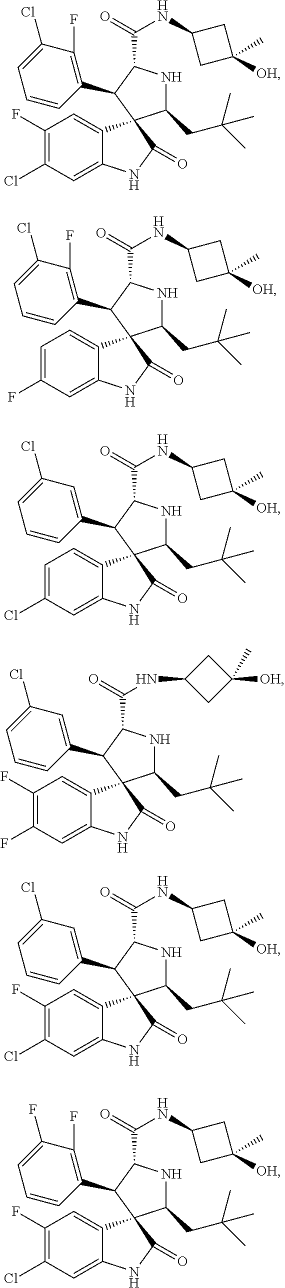 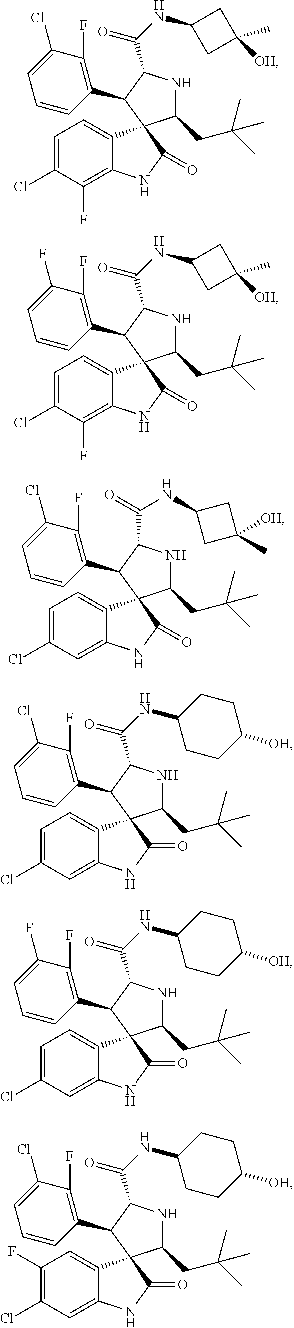 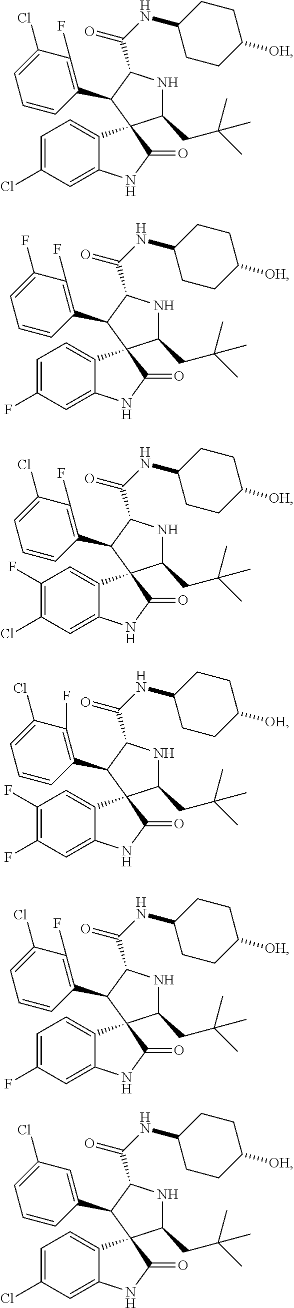 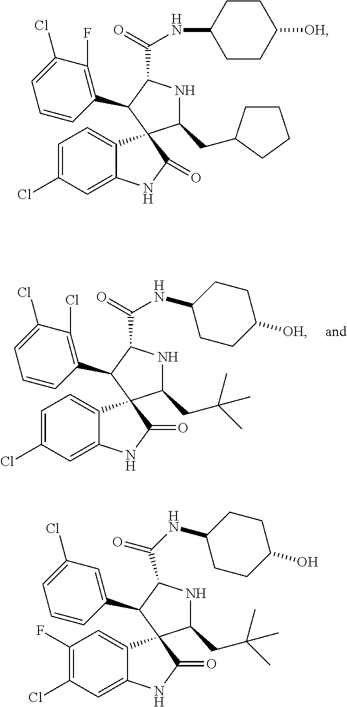
or a pharmaceutically acceptable salt thereof, to the patient, wherein cells of the patient contain a FLT3-ITD mutation.

16. A method of selecting a human patient having acute myeloid leukemia for treatment with a compound selected from the group consisting of: 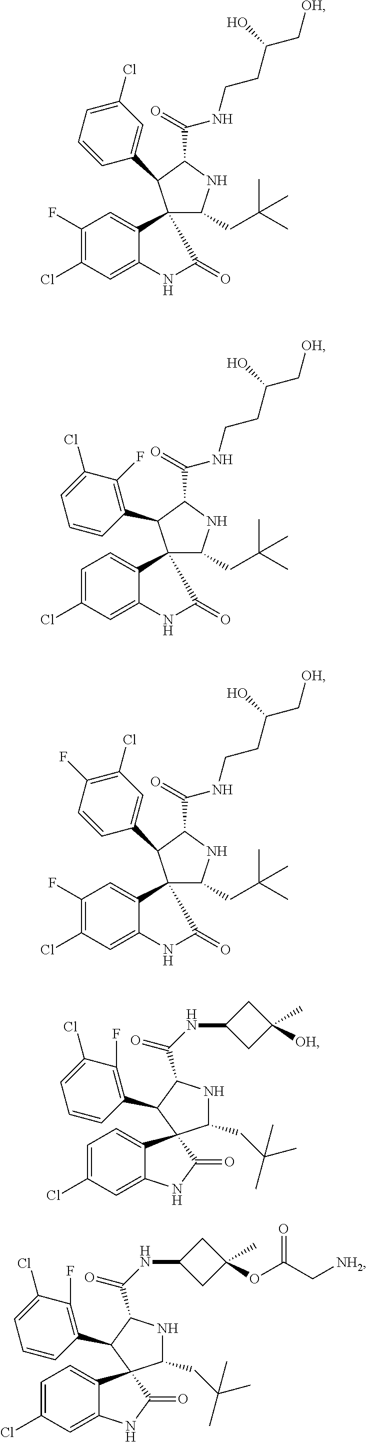 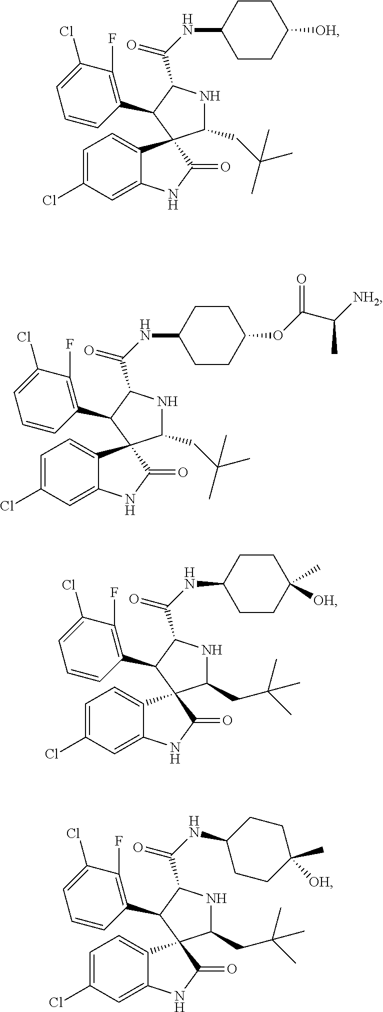 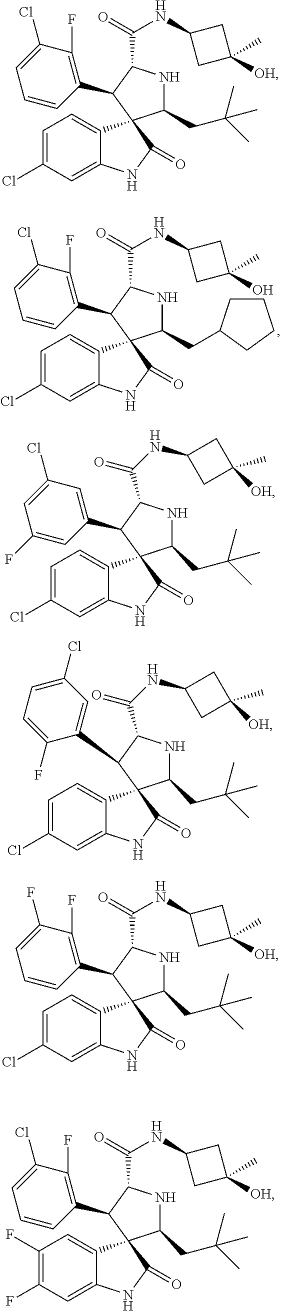 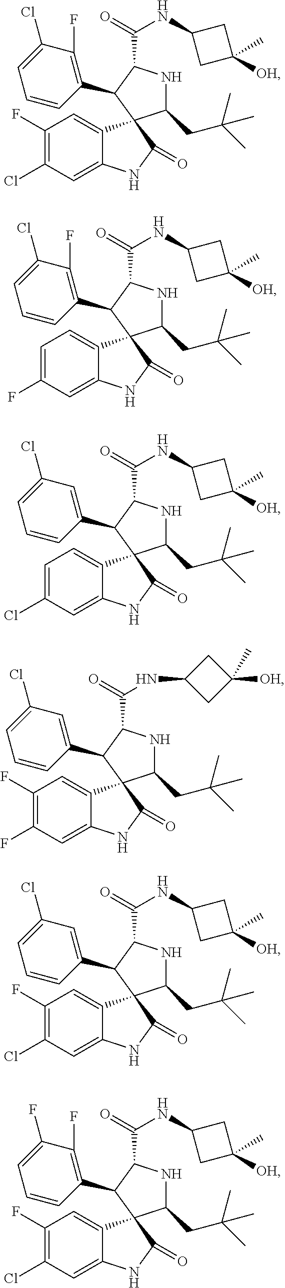 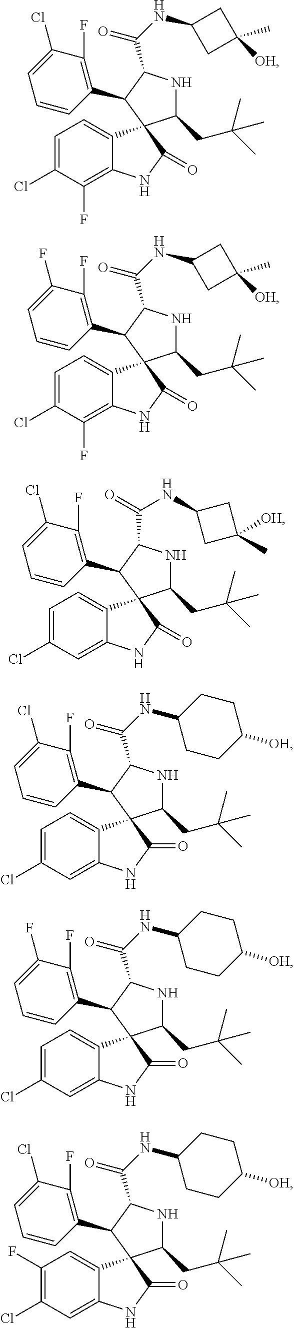 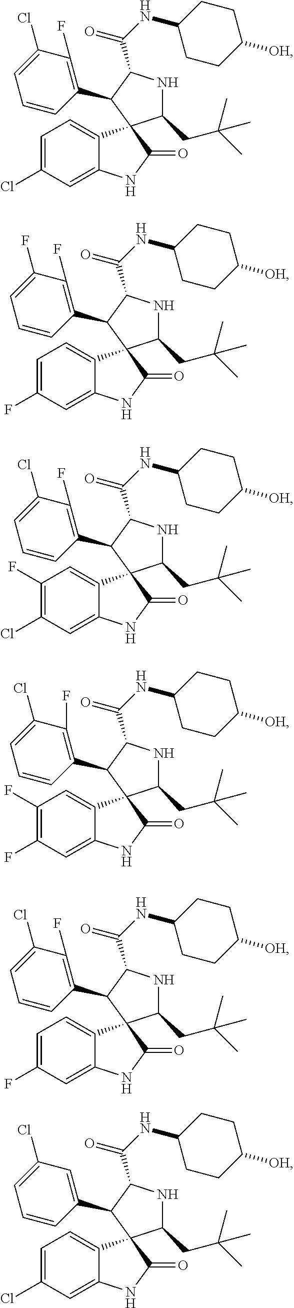 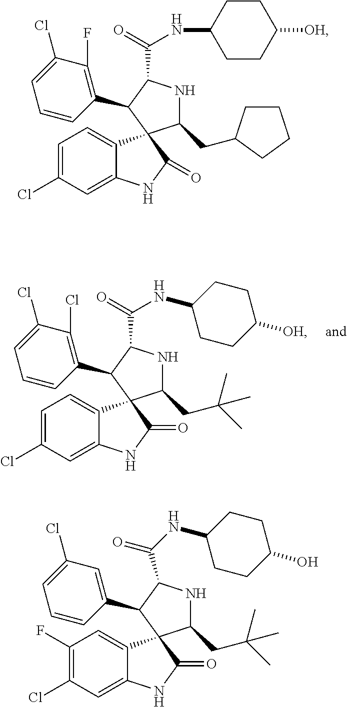
or a pharmaceutically acceptable salt thereof, the method comprising:
(a) obtaining a biological sample from the patient;
(b) determining whether the biological sample contains a FLT3-ITD mutation; and
(c) selecting the patient for treatment if the biological sample contains a FLT3-ITD mutation.

17. A method of predicting treatment outcome in a human patient having acute myeloid leukemia, the method comprising:
(a) obtaining a biological sample from the patient; and
(b) determining whether cells of the patient contain a FLT3-ITD mutation;
wherein the detection of a FLT3-ITD mutation indicates that administering a therapeutically effective amount of a compound selected from the group consisting of: 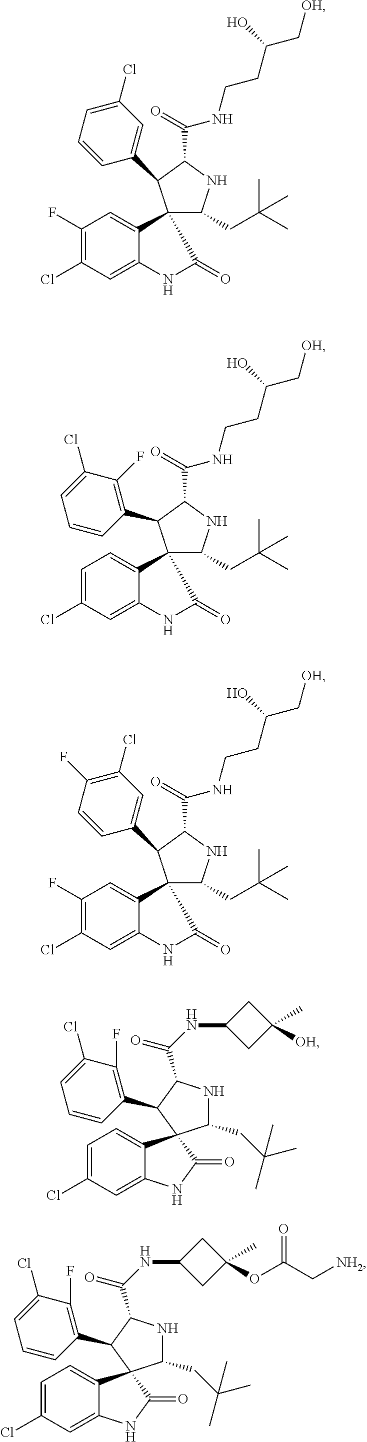 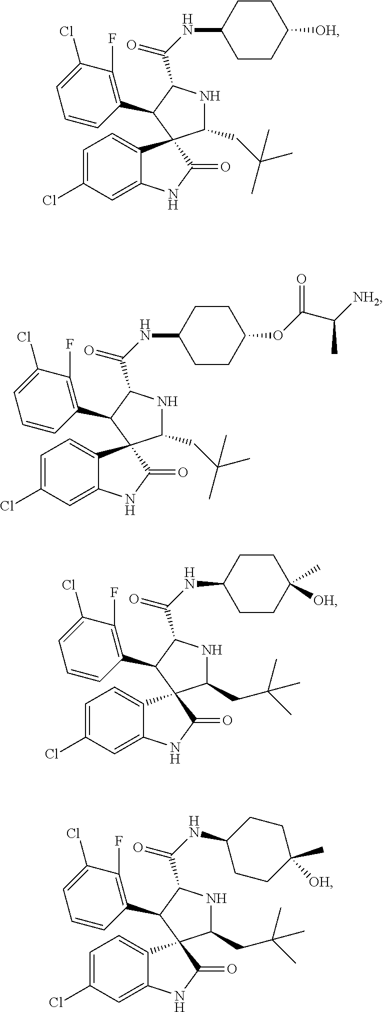 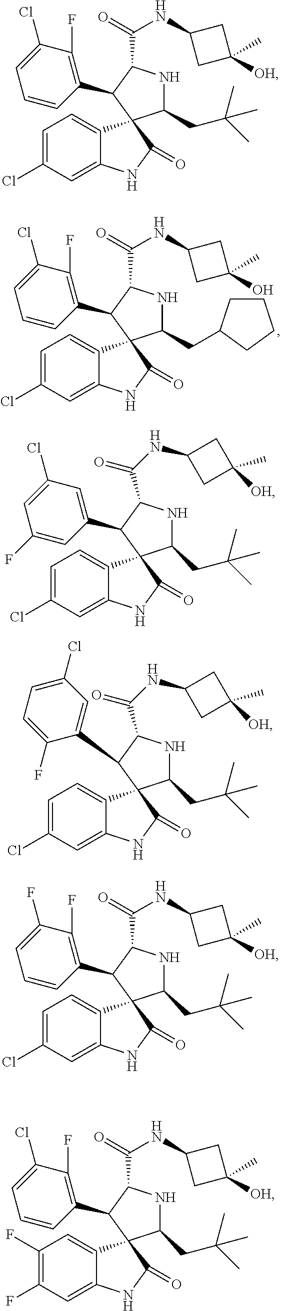 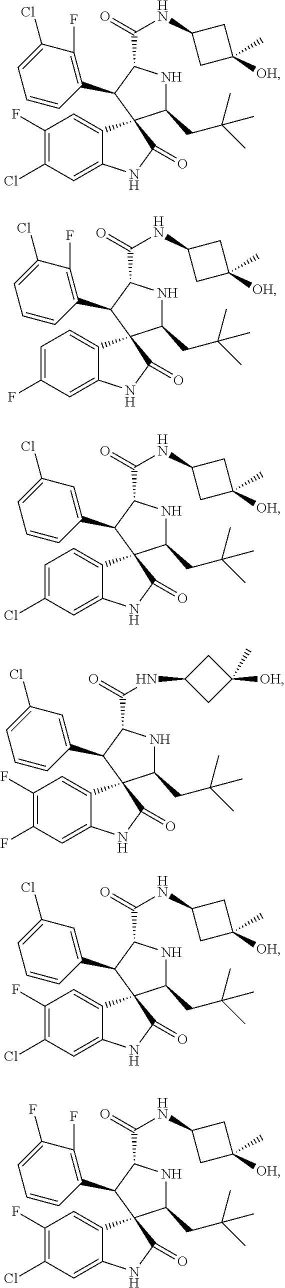 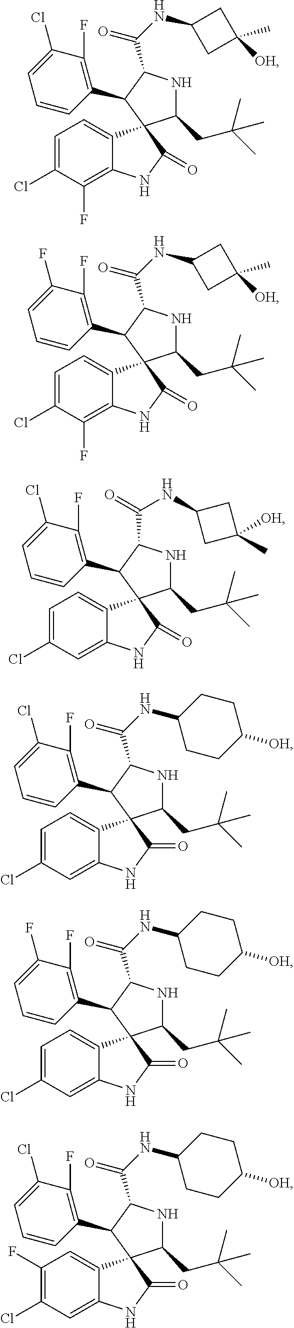 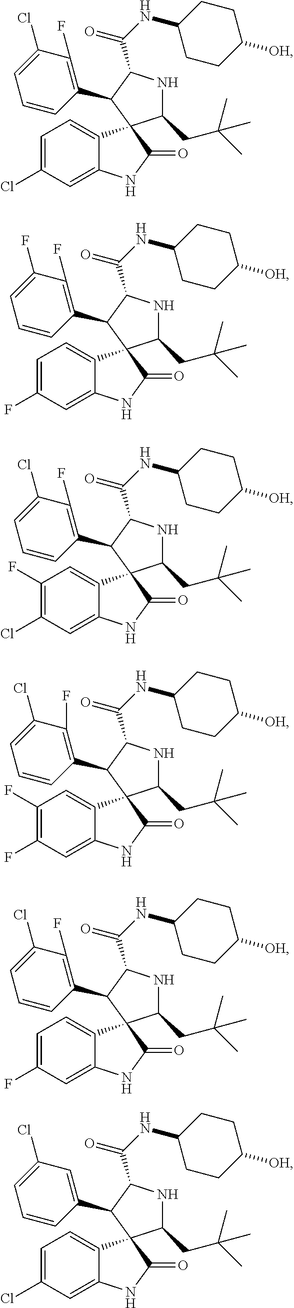 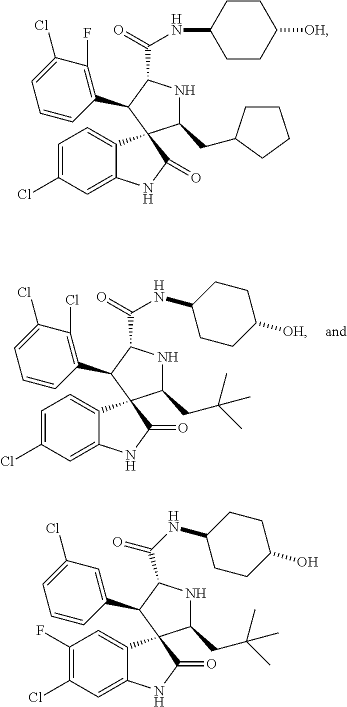
or a pharmaceutically acceptable salt thereof, to the patient will cause a favorable therapeutic response.

18. A method of treating a human patient having acute myeloid leukemia, the method comprising:
(a) obtaining a biological sample from the patient;
(b) determining whether to biological sample contains a FLT3-ITD mutation; and
(c) administering to the patient a therapeutically effective amount of the compound: 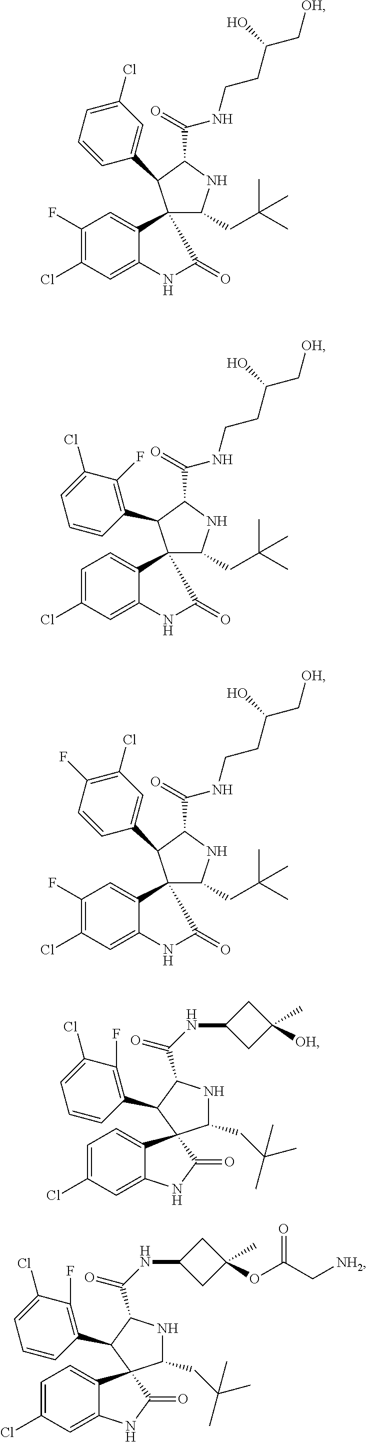 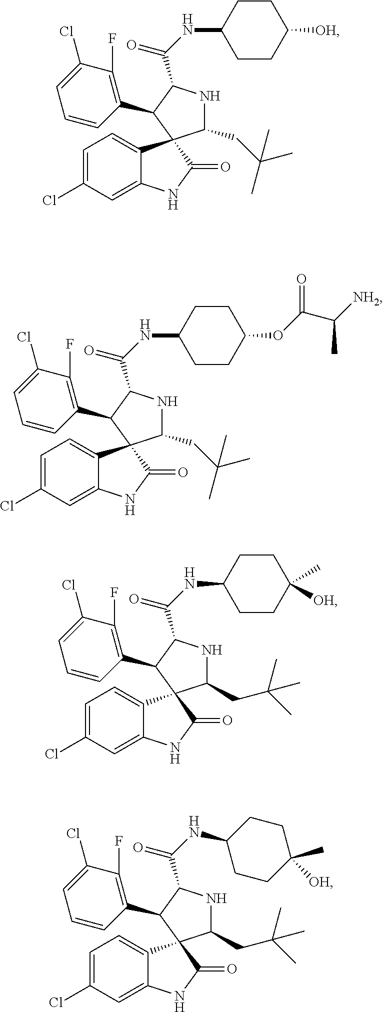 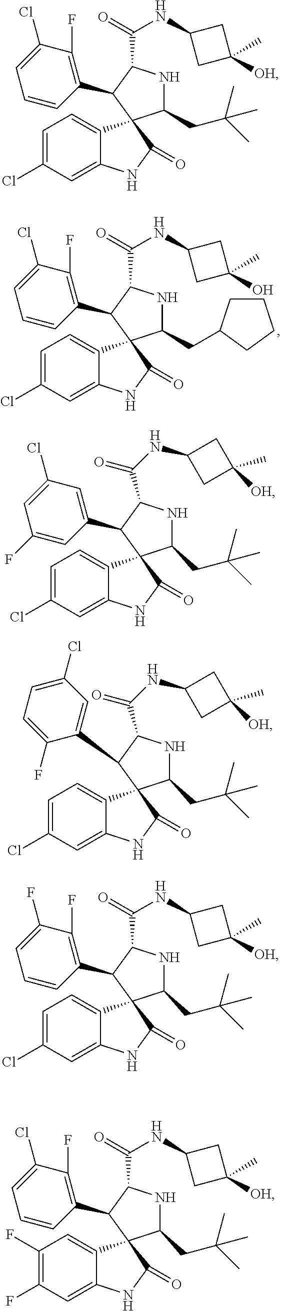 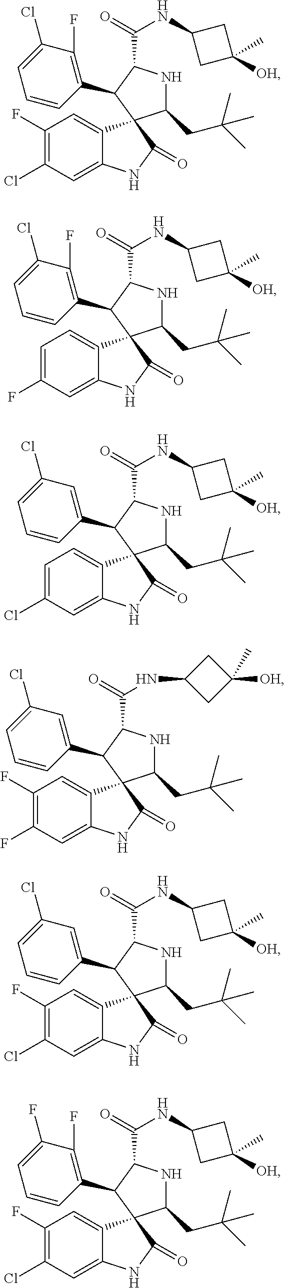 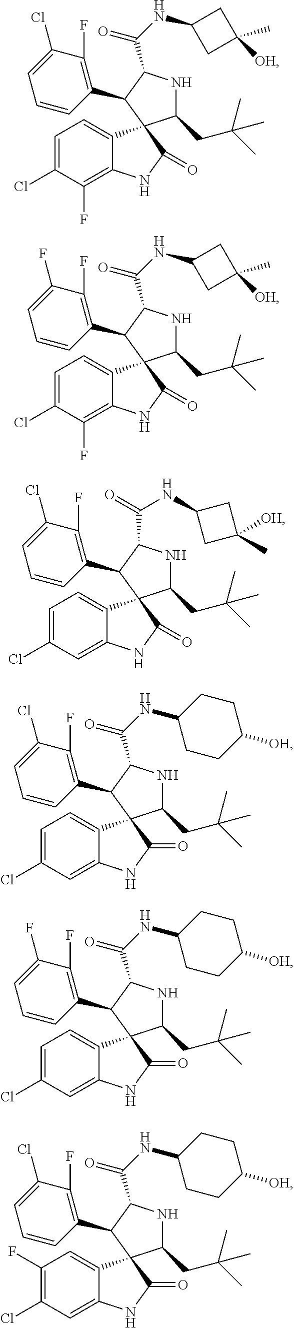 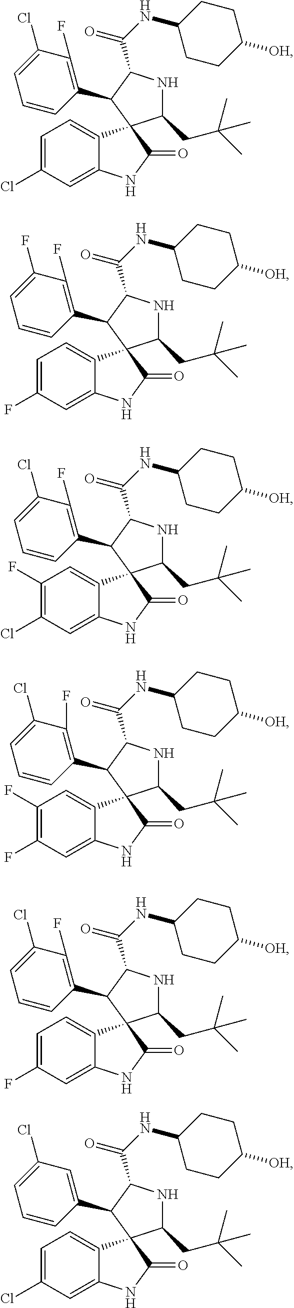 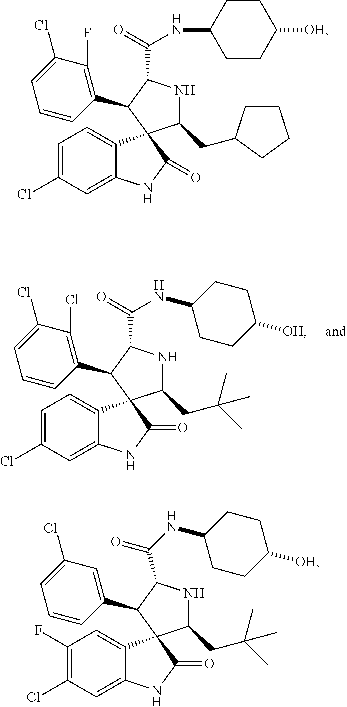
or a pharmaceutically acceptable salt thereof, if the biological sample contains a FLT3 -ITD mutation.


SEQUENCE LISTING (4 sequences)

SEQ ID NO: 1 - DNA, 8 bp, Homo sapiens
gcgaggta

SEQ ID NO: 2 - DNA, 7 bp, Artificial Sequence
  Synthetic
gcgagga

SEQ ID NO: 3 - DNA, 12 bp, Homo sapiens
ccctatgagccg

SEQ ID NO: 4 - DNA, 11 bp, Artificial Sequence
  Synthetic
ccctatggccg